(19) United States
(12) Patent Application Publication
(10) Pub. No.: US 2025/0243514 A1
(43) Pub. Date: Jul. 31, 2025

(54) COMPOSITIONS, METHODS, AND SYSTEMS FOR DNA MODIFICATION

(71) Applicant: The Trustees of Columbia University in the City of New York, New York, NY (US)

(72) Inventors: Samuel Henry Sternberg, New York, NY (US); Chance Meers, New York, NY (US); George Davis Lampe, New York, NY (US); Rimante Zedaveinyte, New York, NY (US)

(21) Appl. No.: 19/177,072

(22) Filed: Apr. 11, 2025

Related U.S. Application Data
(63) Continuation of application No. PCT/US2023/076608, filed on Oct. 11, 2023.
(60) Provisional application No. 63/584,414, filed on Sep. 21, 2023.
(60) Provisional application No. 63/489,495, filed on Mar. 10, 2023.
(60) Provisional application No. 63/379,082, filed on Oct. 11, 2022.

(51) Int. Cl. C12N 15/90; C12N 9/22; C12N 15/11

(52) U.S. Cl. CPC C12N 15/902; C12N 9/22; C12N 15/111; C07K 2319/09; C12N 2310/20

(57) ABSTRACT

The present disclosure provides systems, compositions, and methods for nucleic acid modification. More particularly, the present disclosure provides systems comprising a TnpA protein, a TnpB protein, an IscB protein, or a combination thereof, and methods using thereof.

### CROSS-REFERENCE TO RELATED APPLICATIONS

This application is a continuation of PCT/US2023/076608, filed Oct. 11, 2023, which claims the benefit of U.S. Provisional Application Nos. 63/379,082, filed Oct. 11, 2022, 63/489,495, filed Mar. 10, 2023, and 63/584,414 filed Sep. 21, 2023, the contents of which are herein incorporated by reference in their entirety.

### STATEMENT REGARDING FEDERALLY SPONSORED RESEARCH OR DEVELOPMENT

This invention was made with government support under GM143924 awarded by the National Institutes of Health, and 2239685 awarded by the National Science Foundation. The government has certain rights in the invention.

### FIELD

The present invention relates to compositions, methods, and systems for DNA modification. In particular the present invention provides compositions, and systems comprising a TnpA protein, a TnpB protein, an IscB protein, or a combination thereof, and methods using thereof.

### SEQUENCE LISTING STATEMENT

The content of the electronic sequence listing titled COLUM_41375_305_SequenceListing.xml (Size: 811,889 bytes; and Date of Creation: Apr. 11, 2025) is herein incorporated by reference in its entirety.

### BACKGROUND

DNA transposition is a ubiquitous phenomenon occurring in all kingdoms of life during which discrete segments of DNA called transposons move from one genomic location to another. Insertion sequences (IS) are the simplest autonomous transposable elements. While they tend to be short (<2.5 kb) and carry only those genes needed for transposition, if placed flanking a DNA segment, many are able to mobilize the intervening genes. ISs can be classified into groups or families based on the general features of their DNA sequences and associated transposases. Insertion sequences of IS200/IS605 family contain the genes for their transposition and its regulation: a TnpA transposase, which is essential for mobilization, and an accessory gene, e.g., TnpB or IscB, which are evolutionary ancestors to CRISPR-Cas9 and Cas12 enzymes. These transposon components offer an expansion on genome editing options.

### SUMMARY

Provided herein are engineered systems comprising a TnpA protein, a TnpB protein, an IscB protein, or a combination thereof. In some embodiments, the systems comprise at least one guide RNA, or one or more nucleic acids encoding thereof, wherein the at least one guide RNA is complementary to at least a portion of a target nucleic acid.

In some embodiments, the systems comprise a TnpA protein, a TnpB protein, an IscB protein, or a combination thereof, or one or more nucleic acids encoding thereof and optionally, at least one guide RNA, or one or more nucleic acids encoding thereof, wherein the at least one guide RNA is complementary to at least a portion of a target nucleic acid.

In some embodiments, the TnpA, TnpB, and IscB protein is derived from Geobacillus stearothermophilus, Clostridium botulinum, Clostridium senegalense, or Clostridioides difficile.

In some embodiments, the TnpA protein, TnpB protein, IscB protein are derived from an IS607-family element. In some embodiments, the TnpA protein, TnpB protein, IscB protein are derived from an IS200/IS605-family element.

In some embodiments, the TnpA protein is a serine-family recombinase. In some embodiments, the TnpA protein is a tyrosine-family recombinase

In some embodiments, the TnpA protein comprises any amino acid sequence having at least 70% identity to any of SEQ ID NO: 11, 21, 25, and 38-41. In some embodiments, the TnpA protein comprises any amino acid sequence of any of SEQ ID NO: 11, 21, 25, and 38-41.

In some embodiments, the TnpB protein comprises any amino acid sequence having at least 70% identity to any of SEQ ID NOs: 1-4, 6-9, 17, 22-24, 30-37, and 42-50. In some embodiments, the TnpB protein comprises any amino acid sequence of any of SEQ ID NOs: 1-4, 6-9, 17, 22-24, 30-37, and 42-50.

In some embodiments, the IscB protein comprises any amino acid sequence having at least 70% identity to any of SEQ ID NO: 5 or 10. In some embodiments, the IscB protein comprises any amino acid sequence of any of SEQ ID NO: 5 or 10.

In some embodiments, the system comprises a TnpA protein having an amino acid sequence with at least 70% identity to any of SEQ ID NO: 11, 21, 25, and 38-41, or a nucleic acid encoding thereof, a TnpB protein having an amino acid sequence with at least 70% identity to any of SEQ ID NOs: 1-4, 6-9, 17, 22-24, 30-37, and 42-50, or a nucleic acid encoding thereof, an IscB protein having an amino acid sequence with at least 70% identity to SEQ ID NO: 5 or 10, or a nucleic acid encoding thereof, or a combination thereof; and optionally, at least one guide RNA, or a nucleic acid encoding thereof, wherein the at least one guide RNA is complementary to at least a portion of a target nucleic acid.

In some embodiments, the system comprises, consists of, or consists essentially of a TnpA protein. In some embodiments, the system comprises, consists of, or consists essentially of a TnpA protein and at least one guide RNA.

In some embodiments, the system comprises, consists of, or consists essentially of a TnpB protein. In some embodiments, the system comprises, consists of, or consists essentially of a TnpB protein and at least one guide RNA.

In some embodiments, the system comprises a TnpA protein and a DNA nuclease capable of inducing site-specific single or double strand breaks, or one or more nucleic acids encoding thereof. In some embodiments, the DNA nuclease is a CRISPR/Cas nuclease, an RNA-guided DNA nuclease encoded by insertion sequences, and/or a homing endonuclease. In some embodiments, the CRISPR/Cas nuclease is Cas9 or Cas 12. In some embodiments, the DNA nuclease encoded by insertion sequences is IscB, IsrB, TnpB, or Fanzor. In some embodiments, the homing endonuclease is ISce-I, ICre-I, or HO.

In some embodiments, the system comprises a TnpA protein and at least one of the TnpB protein or IscB protein, or one or more nucleic acids encoding thereof.

In some embodiments, the system further comprises at least one guide RNA.

In some embodiments, the at least one guide RNA comprises a scaffold sequence capable of associating with the TnpA, TnpB, IscB protein, or combination thereof and a guide sequence complementary to at least a portion of a target nucleic acid. In some embodiments, the at least one guide RNA is provided on an omega RNA. In select embodiments, the at least one guide RNA or omega RNA is synthetic.

In some embodiments, the TnpA protein, TnpB protein, and/or IscB protein are at least partially catalytically inactivated. In some embodiments, the TnpA protein, TnpB protein, and/or IscB protein are fused to an effector polypeptide. In some embodiments, the effector polypeptide is a nuclease, a recombinase, an epigenetic modifier, a transposase, an integrase, a resolvase, an invertase, a protease, a DNA methyltransferase, a DNA demethylase, a histone acetylase, a histone deacetylase, a transcriptional repressor, a transcriptional activator, a DNA binding protein, a transcription factor recruiting protein, a deaminase, dismutase, a polymerase, a ligase, a helicase, a photolyase, a glycosylase, or any combination thereof.

In some embodiments, any or all of the TnpA protein, TnpB protein, and IscB protein comprise at least one nuclear localization sequence (NLS).

In some embodiments, the TnpA protein, TnpB protein, IscB protein and the at least one guide RNA are encoded by one, two, three, or four nucleic acids. In some embodiments, the one or more nucleic acids comprises one or more messenger RNAs, one or more vectors, or a combination thereof.

In some embodiments, the system further comprises a target nucleic acid.

In some embodiments, the target nucleic acid is flanked on the 5′ end by a transposon-adjacent motif (TAM) sequence. In some embodiments, the target nucleic acid is flanked on the 3′ end by a transposon-encoded motif (TEM) sequence. In some embodiments, the TAM sequence is TT(C/T)A(A/T/C). In some embodiments, the TAM sequence is TTTAT or TTCAT. In some embodiments, the TAM sequence comprises TGG.

In some embodiments, the system further comprises a donor nucleic acid. In some embodiments, the donor nucleic acid is flanked by at least one of a left end sequence and a right end sequence. In some embodiments, the donor nucleic acid is embedded in a group I self-splicing intron. In some embodiments, the donor nucleic acid is an engineered group I intron comprising an exogenous cargo nucleic acid sequence. In some embodiments, the group I intron is derived from C. botulinum.

In some embodiments, the system is a cell-free system.

Also provided herein are compositions and cells comprising the disclosed systems. In some embodiments, the cell is a prokaryotic cell. In some embodiments, the cell is a eukaryotic cell. In some embodiments, the cell is a mammalian cell. In some embodiments, the cell is a human cell.

Further provided are methods for DNA modification comprising contacting a target nucleic acid sequence with a system disclosed herein. In some embodiments, the modification comprises cleavage of the target nucleic acid, excision of the target nucleic acid, integration of a donor nucleic acid, or a combination thereof.

In some embodiments, the target nucleic acid sequence is flanked on the 5′ end by a transposon-adjacent motif (TAM) sequence. In some embodiments, the target nucleic acid sequence is flanked on the 3′ end by a transposon-encoded motif (TEM) sequence. In some embodiments, the TAM sequence is TT(C/T)A(A/T/C). In some embodiments, the TAM sequence is TTTAT or TTCAT. In some embodiments, the TAM sequence comprises TGG.

In some embodiments, the donor nucleic acid is flanked by at least one of a left end sequence and a right end sequence. In some embodiments, the donor nucleic acid is embedded in a group I intron. In some embodiments, the donor nucleic acid is an engineered group I intron comprising an exogenous cargo nucleic acid sequence. In some embodiments, the group I intron is self-splicing. In some embodiments, the group I intron is derived from an IS607 element. In some embodiments, the group I intron is derived from C. botulinum.

In some embodiments, the target nucleic acid sequence is in a cell and the contacting a target nucleic acid sequence comprises introducing the system into the cell. In some embodiments, the cell is a prokaryotic cell. In some embodiments, the cell is a eukaryotic cell. In some embodiments, the cell is a mammalian cell. In some embodiments, the cell is a human cell.

In some embodiments, the introducing the system into the cell comprises administering the system to a subject. In some embodiments, the subject comprises a disease or disorder. In some embodiments, the methods comprise treating or preventing a disease or disorder in subject comprising administering an effective amount of the system disclosed herein to the subject in need thereof.

Other aspects and embodiments of the disclosure will be apparent in light of the following detailed description.

### BRIEF DESCRIPTION OF THE DRAWINGS

FIGS. 1A-1D show the distribution of IS200/IS605-like elements in Geobacillus stearothermophilus. FIG. 1A, Schematic of a representative IS200/1S605 element. TnpA encodes a Y1-family tyrosine transposase responsible for DNA excision and integration; tnpB/iscB encode RNA-guide nucleases whose biological roles are unknown. FIG. 1B, Schematic of a non-autonomous IS element encoding TnpB and its associated overlapping ωRNA; a structural covariation model is shown in the inset. The green rectangle indicates the transposon boundaries, and the guide portion of the ωRNA is shown in blue. LE and RE, transposon left end and right end. FIG. 1C, Genome-wide distribution of IS200/IS605-like elements in G. stearothermophilus strain DSM458. Five distinct families are shown (ISGst1-5), based on sequence similarity of transposon ends and nuclease encoded. FIG. 1D, Read coverage from small RNA-seq data of Gst strain ATCC 7953, demonstrating expression of putative ωRNAs from each of the indicated ISGst families. TnpB-associated ωRNAs are encoded within/downstream of the ORF, whereas IscB-associated ωRNAs are encoded upstream of the ORF.

FIGS. 2A-2F show TnpA catalyzes DNA excision for multiple families of IS elements. FIG. 2A, Schematic of ISGst2 element, highlighting the subterminal palindromic transposon ends located on the top strand (top). Transposon-adjacent and transposon-encoded motifs (TAM and TEM) are highlighted in yellow and orange respectively, DNA guides are shown in red, and their putative base-pairing interactions are indicated; dotted lines indicate transposon boundaries and thus the sites of ssDNA cleavage and re-ligation. The donor joint formed upon transposon loss is shown at the bottom and comprises the TAM abutting RE-flanking sequence (denoted with N's). LE is SEQ ID NO: 204; RE is SEQ ID NO: 205. FIG. 2B, Schematic of heterologous transposon excision assay in E. coli. Plasmids encode TnpA and mini-transposon (Mini-Tn) substrates, whose loss is monitored by PCR using the indicated primers. FIG. 2C, TnpA is active in recognizing and excising all five families of ISGst elements, as assessed by analytical PCR. Cell lysates were tested after overnight expression of TnpA with the indicated ISGst mini-Tn substrates, and PCR products were resolved by agarose gel electrophoresis. Marker denotes a positive excision control; U, unexcised; E, excised; M denotes a Y125A TnpA mutant. FIG. 2D, Excision products from c exhibit the expected ‘donor joint’ architecture, as demonstrated by Sanger sequencing. Dotted lines denote the re-ligation site following excision; the TAM is highlighted. SEQ ID NOs: 206-210 for ISGst1, ISGst2, ISGst3, ISGst4, and ISGst5, respectively. FIG. 2E, Transposon excision requires intact LE and RE sequences, as seen via testing of the mutagenized mini-Tn substrates indicated on the right. Experiments were performed as in c using ISGst2. Transposon ends and TAMs are indicated with green triangles and yellow boxes, respectively; M denotes a Y125A TnpA mutant. FIG. 2F, Transposon excision is dependent on cognate pairing between compatible TAM and guide sequences. Excision experiments were performed as in FIG. 2C using ISGst2 with the indicated mutations in the TAM/TEM (blue) or DNA guide (red). Substrate 4 has mutations to cognate sequences derived from IS608.

FIGS. 3A-3H show TnpB and IscB target ‘donor joint’ molecules excised by TnpA. FIG. 3A, Schematic representation of each IS family (colored rectangle), alongside homologous sites from related Gst strains that lack the transposon insertion. TAMs are highlighted in the donor joint sequences (SEQ ID NOs: 211-214 for ISGst2, ISGst3, ISGst4, and ISGst5, respectively) shown below each element. FIG. 3B, Schematic of E. coli-based plasmid interference assay. Protein-RNA complexes are encoded by pEffector, and targeted cleavage of pTarget results in a loss of kanamycin resistance and cell lethality on selective LB-agar plates. FIG. 3C, G. stearothermophilus TnpB and IscB homologs are highly active for RNA-guided DNA cleavage, as assessed by plasmid interference assays. Transformants with a targeting (T) or non-targeting (NT) ωRNA-pTarget combination were serially diluted and plated on selective media at 37° C. for 24 h. FIG. 3D, Quantification of the data in FIG. 3C, normalized to the non-targeting (NT) plasmid control for each ISGst element. CFU, colony forming units; ND, not detected. FIG. 3E, DNA cleavage by TnpB2 is highly sensitive to TAM mutations, as assessed by plasmid interference assays. Data were quantified and plotted as in FIG. 3D for the indicated TAM mutations; TTTAT denotes the WT TAM. FIG. 3F, DNA cleavage by IscB is highly sensitive to TAM mutations, as assessed by plasmid interference assays. Data were quantified and plotted as in FIG. 3D for the indicated TAM mutations; TTCAT denotes the WT TAM. FIG. 3G, Schematic of E. coli-based genome targeting assay, in which RNA-guided DNA cleavage of lacZ by TnpB/IscB results in cell death. FIG. 3H, TnpB2 and IscB are active for targeted genomic DNA cleavage, as assessed by genome targeting assay. Transformants with a targeting (T) or non-targeting (NT) ωRNA were serially diluted and plated on selective media at 37° C. for 24 h. dTnpB2, D196A mutation; dIscB, D58A/H209A/H210A mutations.

FIGS. 4A-4D show unbiased identification of TnpB/IscB TAM specificity by ChIP-seq and library assays. FIG. 4A, Schematic of ChIP-seq workflow to monitor genome-wide binding specificity of TnpB/IscB. E. coli cells were transformed with plasmids encoding catalytically inactive dTnpB2 or dIscB and a genome targeting (T) or non-targeting (NT) ωRNA. After induction, cells were harvested, protein-DNA cross-links were immunoprecipitated, and NGS libraries were prepared and sequenced. FIG. 4B, (Left) Genome-wide representation of ChIP-seq data for dIscB with target site (blue triangle) shown, for T and NT samples alongside the input control. Coverage is shown as reads per kilobase per million mapped reads (RPKM), normalized to the highest peak in the T sample. (Right) Off-target binding events were analyzed by MEME ChIP, which revealed a strongly conserved consensus motif consistent with the WT TAM (TTCAT) but weak seed sequence bias; part of the ωRNA guide sequence is shown below. Consensus motifs are oriented 5′ of the IS element left end. n, number of peaks contributing to the motif; E, E-value significance. FIG. 4C, Representative ChIP-seq data for dTnpB2, plotted as in FIG. 4B. FIG. 4D, (Left) Schematic of TAM library cleavage assay, in which plasmids expressing nuclease-active TnpB/IscB and an associated ωRNA (pEffector) are designed to cleave a target sequence flanked by randomized 6-mer (pTarget). Plasmid cleavage results in plasmid elimination, loss of cell viability, and depletion of the particular TAM upon library sequencing. (Right) WebLogo representation of the 10-most depleted sequences upon deep sequencing of plasmid samples from the TAM library cleavage assay for TnpB2 and IscB Consensus motifs are oriented 5′ of the IS element left end.

FIGS. 5A-5E show RNA-guided nucleases preserve IS elements at the donor site following transposase-mediated excision. FIG. 5A, Schematic of experimental workflow to measure transposon fate in E. coli in the presence of TnpA and TnpB. A mini-Tn was inserted at a compatible TAM site in lacZ, such that cells grown on X-gal exhibit a blue colony phenotype upon permanent transposon excision, or a white colony phenotype if the transposon is retained. Cells were transformed with plasmids expressing WT or mutant TnpA and/or TnpB2, with a targeting (T) or non-targeting (NT) ωRNA. Kanamycin-resistant cells with a blue color phenotype will result upon transposon loss at the donor site (excision) and transposon gain at a new target site (integration). FIG. 5B, TnpB promotes robust transposon retention at the donor site, as assessed by blue-white colony screening. Representative plating results are shown from experiments that included the indicated components. M, TnpA (Y125A) mutant; dTnpB, D196A mutation. FIG. 5C, Quantification of the data from FIG. 5B across multiple experimental replicates. Green bars indicate the frequency of blue colonies as a measure of transposon excision/retention for the indicated experimental conditions and pink indicates the frequency of blue colonies that maintain kanamycin resistance as a measure of transposon excision and reintegration elsewhere in the genome. Bars indicate mean±standard deviation (n=3). FIG. 5D, Genotypes inferred from blue-white colony screening were assessed by PCR analysis and agarose gel electrophoresis for the indicated experimental conditions, which reports on whether the mini-Tn is unexcised (UE) or excised (E) at the donor lacZ site. The first two lanes denote marker controls (ME: Mock excised and MU: Mock Unexcised) for the two possible PCR products. FIG. 5E, Peel-and-paste/cut-and-copy model for how TnpA and TnpB/IscB coordinate their catalytic activities to maintain the presence of IS200/605-family transposons at donor sites. TnpA mediates excision and re-ligation of flanking sequences at the donor site as ssDNA becomes available during DNA replication, resulting in transposon loss from the donor site. The excised ssDNA product is concurrently ligated to form a circular ssDNA-transposome complex, which can be reintegrated downstream of a TAM motif elsewhere in the genome, albeit at much lower efficiency than excision. In the presence of TnpB/IscB, RNA-guided DNA cleavage of the donor joint initiates homologous recombination with the sister chromosome that still contains the IS element, thus rapidly restoring the transposon at the original donor site; the absence of TnpB/IscB leads to permanent transposon loss after cell division. TnpB/IscB can also cleave sister chromosomes lacking the newly integrated IS element after transposition to a new target site, facilitating further spread. The transposon is shown in dark blue; the TAM is shown in yellow, and light blue rectangles represent regions complementary to the guide portion of the ωRNA.

FIGS. 6A-6D show bioinformatic analyses of IscB and TnpB homologs. FIG. 6A, Phylogenetic tree of IscB and IsrB protein homologs; IscB contain HNH and RuvC nuclease domains, whereas IsrB lacks the HNH nuclease. Genetic neighborhood analyses demonstrate that most homologs are encoded proximal to a predicted ωRNA (inner ring), whereas the vast majority do not reside near a predicted TnpA transposase gene (outer ring). The GstIscB homolog used in this study is indicated. Bootstrap values are indicated for major nodes. FIG. 6B, Schematic of a non-autonomous IS element encoding IscB and its associated ωRNA; a structural covariation model is shown in the inset. The red rectangle and dotted black line indicate the transposon boundaries, and the guide portion of the ωRNA is shown in blue. LE and RE, transposon left end and right end. FIG. 6C, Orientation bias of the nearest upstream ORFs to the indicated protein-coding gene (iscB, tnpB or IS630), demonstrating that IS elements encoding IscB are preferentially integrated (or retained) in an orientation matching that of the upstream gene. The y-axis indicates the frequency of ORFs containing the same orientation, at a distance from the gene start codon defined by the x-axis. 242 bp represents the average length of IscB-associated ωRNAs upstream of IscB ORF. The spike at ˜0-bp for TnpB corresponds to IS elements that encode adjacent/overlapping tnpA and tnpB genes. IS630 transposase genes are included as a representative gene from unrelated transposable elements. FIG. 6D, Phylogenetic tree of TnpB homologs. Genetic neighborhood analyses demonstrate that most homologs are encoded proximal to a predicted ωRNA (inner ring), whereas the vast majority do not reside near a predicted TnpA transposase gene (outer rings). Bootstrap values are indicated for major nodes. Interestingly, TnpB homologs are associated with two unrelated transposase families, tyrosine transposases (TnpA (Y)) and serine transposases (TnpA(S)) in bacteria. GstTnpB homologs used in this study are highlighted, along with the predicted structures of their associated ωRNAs, based on covariance modeling. ISGst1 TnpB1 was not experimentally active and ωRNA did not show strong covariation in structure and was therefore omitted.

FIGS. 7A-7E show classification of IS605-family elements encoded by G. stearothermophilus strain DSM458. FIG. 7A, DNA multiple sequence alignment of transposon left ends for IS200/IS605-family elements from G. stearothermophilus. The weblogo (top) is built from 47 unique elements and one representative sequence from each family (SEQ ID NOS: 215-219 for ISGst1, ISGst3, ISGst2, ISGst5, and ISGst4, respectively) is shown below, with the TAM shown in yellow and DNA guide sequences shown in red as indicated. Nucleotides highlighted in black exhibit covarying mutations, relative to ISGst1. TAM, transposon-adjacent motif; dotted black line indicates the transposon boundary. FIG. 7B, DNA multiple sequence alignment (SEQ ID NOs: 220-224 for ISGst1, ISGst3, ISGst2, ISGst5, and ISGst4, respectively) of transposon right ends for IS200/IS605-family elements from G. stearothermophilus, shown as in FIG. 7A. TEM, transposon-encoded motif is shown in orange. FIG. 7C, Phylogenetic tree of ISGst elements based on the transposon left end. Each colored clade encodes an associated TnpB/IscB protein homolog and is flanked by the indicated TAMs sequence. FIG. 7D, Phylogenetic tree of ISGst elements based on the transposon right end, shown as in FIG. 7B but with TEM sequence in lieu of TAM. FIG. 7E, Schematic of PATEs (palindrome associated transposable elements) related to ISGst1 and ISGst5, which contain similar transposon ends but no protein-coding genes. The percent sequence identity between shaded regions (black) is shown, as are the genomic accession IDs and coordinates.

FIGS. 8A-8G show specificity and efficiency of transposon DNA excision by TnpA. FIG. 8A, Schematic of heterologous transposon excision assay in E. coli. Plasmids encode TnpA and mini-transposon (Mini-Tn) substrates, whose loss is monitored by PCR using the indicated primers. The expected sizes of PCR products generated from donor joints that are produced upon re-ligation of flanking sequences are shown, for both ISGst1 and H. pylori IS608. FIG. 8B, TnpA homologs do not cross-react with distinct IS elements, as assessed by analytical PCR. Cell lysates were tested after overnight expression of TnpA in combination with a mini-Tn substrate, from either G. stearothermophilus (G) or H. pylori (H), and PCR products were resolved by agarose gel electrophoresis. M refers to catalytically inactive mutants. Note that HpyTnpA is substantially more active for DNA excision than GstTnpA under the tested conditions. U, unexcised; E, excised. FIG. 8C, Schematic of qPCR assay to quantify excision frequencies, in which one of the two primers anneals directly to the donor joint formed upon mini-Tn excision and re-ligation. FIG. 8D, Comparison of simulated excision frequencies, generated by mixing clonally excised and unexcised lysate in known ratios, versus experimentally determined integration efficiencies measured by qPCR. FIG. 8E, qPCR-based quantification of TnpA-mediated excision of an ISGst1 mini-Tn substrate in E. coli. Mock refers to a cloned excision product; M denotes a TnpA mutant (Y125A); ND, not detected above a 0.0001% threshold. Bars indicate mean±standard deviation (n=3). FIG. 8F, Schematic of mini-Tn ISGst2 element, highlighting the subterminal palindromic transposon ends located on the top strand (top). Transposon-adjacent and transposon-encoded motifs (TAM and TEM) are shown in yellow and orange, respectively; DNA guides are shown in red, and their putative base-pairing interactions are indicated; dotted lines indicate transposon boundaries and thus the sites of ssDNA cleavage and re-ligation. LE is SEQ ID NO: 204; RE is SEQ ID NO: 205. Sanger sequencing (SEQ ID NO: 225) of excision events confirm the identity of the expected donor joint product formed upon transposon loss (bottom). Sanger sequencing results (SEQ ID NO: 227) are duplicated from FIG. 2D. FIG. 8G, Schematic and Sanger sequencing data as in FIG. 8F, but for a modified ISGst2 substrate containing TEM mutations. LE is SEQ ID NO: 204; RE is SEQ ID NO: 226. Experimentally detected products erroneously excise at an alternative TEM-like sequence located outside of the native transposon boundary (orange), presumably because of the need to maintain cognate base-pairing between the DNA guide and TEM.

FIGS. 9A-9C show mating-out assay to monitor transposition of ISGst2. FIG. 9A, Schematic of mating-out assay, in which transposition events into the F-plasmid are monitored via drug selection. E. coli donor cells carrying an F-plasmid were transformed with a plasmid encoding TnpA and ISGst2-derived mini-Tn. After induction of TnpA, conjugation was used to transfer the F-plasmid into the recipient strain, and transposition events were quantified by selecting for recipient cells (Rif^{R}) containing spectinomycin (F⁺) and kanamycin (mini-Tn⁺) resistance. FIG. 9B, Transposition frequency of ISGst2 into the F-plasmid was measured with and without tnpA. Bars indicate mean±standard deviation (n=6). FIG. 9C, Drug-selected cells from mating-out assays contain TAM-proximal IS insertions, as evidenced by long-read Nanopore sequencing. A genetic map of the F-plasmid is shown, along with the location of distinct ISGst2-derived mini-Tn integration events. The insets show a zoom-in view of each integration site at the nucleotide level, with the TAM motif highlighted in yellow and the integration site specified by an arrow. SEQ ID NOs: 228 and 229 for insertion site 1; SEQ ID NOs: 230 and 231 for insertion site 2; SEQ ID NOs: 232 and 233 for insertion site 3; SEQ ID NOs: 234 and 235 for insertion site 4.

FIGS. 10A-10E show DNA cleavage parameters with TnpB/IscB nucleases. FIG. 10A, Promoter screen to optimize conditions for E. coli-based interference assays using plasmid-encoded ωRNA and TnpB2. P1 indicates promoters for ωRNA expression, P2 indicates promoters for TnpB2 expression. Transformants with a targeting (T) or non-targeting (NT) ωRNA-pTarget combination were serially diluted and plated on selective media at 37° C. for 24 h. FIG. 10B, Results from plasmid interference assays with HpyTnpB (IS608) and DraTnpB (ISDra2) using ωRNAs that target native donor joint products, which revealed an absence of activity for HpyTnpB. Experiments were performed as in FIG. 10A. FIG. 10C, DNA cleavage by TnpB2 is highly sensitive to TAM mutations, as assessed by plasmid interference assays. Data are shown as in FIG. 10A, with the indicated TAM sequences; TTTAT denotes the WT TAM, and NT denotes a non-targeting control. FIG. 10D, DNA cleavage by IscB is highly sensitive to TAM mutations, as assessed by plasmid interference assays. Data are shown as in FIG. 10A, with the indicated TAM sequences; TTCAT denotes the WT TAM, and NT denotes a non-targeting control. FIG. 10E, TnpB2 is only active for targeted genomic DNA cleavage using select ωRNAs, as assessed by genome targeting assays. Transformants with a non-targeting (NT) or one of three lacZ-specific guides were serially diluted and plated on selective media at 37° C. for 24 h.

FIGS. 11A-11D show off-target ChIP-seq DNA binding analyses. FIG. 11A, ChIP-seq experiments reveal recruitment of dIscB to the target site (blue triangle) with a targeting ωRNA shown as two independent reps. Genome-wide representation of ChIP-seq data for dIscB reshown from FIG. 4B with addition of second replicate. Representative off-target sites for dIscB identified by MACS3 are highlighted (OT1-4) and analyzed in middle and right panels, respectively. Middle panel highlights analysis of off-target binding events by dIscB using MEME ChIP, as shown in FIG. 4B. Motifs shared by off-target peaks reveal conserved TAM sequences and little conservation of the adjacent seed sequence (left; SEQ ID NOs: 236-240 for On, OT1, OT2, OT3, and OT4, respectively). The sequence of the 5′ end of the corresponding ωRNA is shown at the bottom of each motif. Two targeting replicates are shown. n indicates the number of peaks contributing to the motif and their percentage of total peaks called by MACS3; E, E-value significance of the motif generated from the MEME ChIP analysis (right of weblogo). DNA sequences corresponding to the on-target and off-target sites are shown on right with TAM (yellow) and mismatches (red) highlighted. OT1-4 represent the top enrichment peaks contributing to each motif, as called by MACS3 with respect to the input sample (Methods). FIG. 11B, ChIP-seq experiments reveal recruitment of dTnpB2 to the target site (blue triangle) with a targeting ωRNA shown as two independent replicates. Data shown as in FIG. 11A. Similar to dIscB, dTnpB2 shows limited seed sequence requirements. SEQ ID NOs: 241-245 for On, OT1, OT2, OT3, and OT4, respectively. FIG. 11C, ChIP-seq experiments reveal recruitment of dCas9 to the target site (blue triangle) with a targeting ωRNA shown as two independent replicates. Data shown as in FIG. 11A. Analysis of off-target sites reveal a short (3-4 nt) seed sequence adjacent to the PAM motif. SEQ ID NOs: 246-250 for On, OT1, OT2, OT3, and OT4, respectively. FIG. 11D, ChIP-seq experiments reveal recruitment of dCas 12a to the target site (blue triangle) with a targeting ωRNA shown as two independent replicates. Data shown as in FIG. 11A. Analysis of off-target sites reveals a short (4-5 nt) seed sequence adjacent to PAM motif. SEQ ID NOs: 251-255 for On, OT1, OT2, OT3, and OT4, respectively.

FIGS. 12A-12C show qPCR analysis of IS element loss upon TnpA and TnpB co-expression. FIG. 12A, Schematic of qPCR-based strategy for quantifying excision. Primers are designed flanking the donor joint following excision and re-ligation. Selective PCR conditions with a shortened extension time allows for reduced amplification of the starting locus containing the mini-Tn. FIG. 12B, Comparison of simulated excision frequencies, generated by mixing clonally excised and unexcised lysate in known ratios, versus experimentally determined integration efficiencies measured by qPCR. FIG. 12C, qPCR-based quantification of transposon excision. Excised represents wild-type lacZ and unexcised represents lacZ containing mini-Tn as controls. TnpA was provided in all conditions shown in green. The detection limit is based on simulated excision frequencies shown in FIG. 12B.

FIG. 13 is a schematic of Clostridium botulinum (Cbo) IStron (CboIStron) and a covariation model of ChoTnpB ωRNA. Green rectangle indicates IStron parts derived from group I intron and shows boundaries of mobile genetic element. Covariation model of ChoTnpB ωRNA is shown in the inset. PKI indicates a possible pseudoknot formation site. Dashed line separating covariation model of ωRNA and the guide sequence indicates 3′ IStron boundary.

FIGS. 14A-14E show ChoTnpB robustly cleaves plasmid DNA in E. coli. FIG. 14A, Schematic of plasmid interference assay in E. coli. Protein-RNA complexes are encoded by pEffector, and targeted cleavage of pTarget results in a loss of kanamycin resistance and cell lethality on selective LB-agar plates. FIG. 14B, CboTnpB actively cleaves DNA when both TAM and target complementary to ωRNA are present. FIG. 14C, ChoTnpB DNA cleavage is dependent on RuvC active site. FIG. 14D, Mature ωRNA species are detected only in the presence of active ChoTnpB, the arrow indicates 5′ processing site. FIG. 14E, ωRNA maturation site shown on the covariation model, cleavage site indicated by the red arrow.

FIGS. 15A-15C show unbiased detection of ChoTnpB TAM. FIG. 15A, Schematic of plasmid interference assay in E. coli. pEffector encoded protein-RNA complexes lead to targeted cleavage of pTargets which have a compatible TAM sequence. This results in a loss of kanamycin resistance and cell lethality on selective LB-agar plates. FIG. 15B, Schematic of the plasmid library targeting. Degenerate 6 nt sequence is located at the 5′ to the guide sequence. Target SEQ ID NOs 256 and 257; Guide SEQ ID NO: 258. FIG. 15C, WebLogo representation of 50 most depleted library members. Consensus motif is located 5′ to the target sequence.

FIGS. 16A-16C show CboIStron actively self-splices in E. coli at the RNA level. FIG. 16A, A model of IStron splicing, leading to its removal from transcribed RNA and relegation of exons. FIG. 16B, Schematic of a minimal IStron construct used for splicing assays in E. coli (top row) and the selected truncations to determine predicted right end structure for splicing. FIG. 16C, RT-PCR gel showing splicing of the minimal IStron and selected right-end truncations.

FIGS. 17A-17B show CboTnpA excises the IStron at the DNA level, and the donor junction is recognized by TnpB. FIG. 17A, A model of IStron mobility mediated by TnpA, showing its excision from native location and integration in a new location. The newly formed donor junction can be recognized by TnpB and either promote recombination of the element back into its previous location or cause the loss of the plasmid due to double-stranded break. FIG. 17B, Excision assay showing that IStron is effectively excised by TnpA, but that the excision product is undetectable in the presence of TnpB.

FIG. 18 is graphs of editing outcomes with TnpB and IscB proteins. Various TnpB and IscB proteins were analyzed in human cells for their potential editing efficiencies at multiple target sites within the HEK3 locus. Each graph reports the DNA editing efficiency for the genome editing reagent shown in the title at the top of the graph; editing efficiencies were calculated as the indel frequency from high-throughput sequencing data, with the aid of CRISPResso2. Cas9 is shown as a positive control (upper left). “NT” represents a non-targeting ωRNA, while “T” represents a targeting ωRNA. “T to G” represents an ωRNA in which the 5′ sequence was extended to the nearest G base, such that the IscB ωRNA expresses a completely complementary ωRNA while still beginning with a “G” for proper U6-based RNA expression to occur.

FIGS. 19A-19E show experiments revealing the TnpA activity in stimulating recombination efficiencies. FIG. 19A, Schematic of experimental workflow to investigate transposon recombination in E. coli in the presence of TnpA and TnpB. A native ISGst2 transposon encoding either TnpB or both TnpA and TnpB was cloned adjacent to a compatible TAM site within plasmid-encoded lacZ, and this plasmid was used to transform E. coli containing an intact lacZ locus. RNA-guided DNA cleavage of genomic lacZ is expected to trigger a recombination event with the plasmid-encoded ISGst2-lacZ element, leading to genomic gain of the transposon and a white-colony phenotype. FIG. 19B, Images of representative LB-agar plates, highlighting the roles of TnpA and TnpB in transposon maintenance and spread via recombination. M, TnpA (Y125A) mutant; dTnpB, D196A mutant. FIG. 19C, Transposon-encoded TnpA and TnpB collaborate to efficiently mobilize themselves into a vacant donor site via recombination. Blue bars report the transformation efficiency for each ISGst2 plasmid, and white bars quantify colonies exhibiting a lacZ-phenotype, suggestive of a recombination (e.g., gene conversion) product. Catalytically active TnpA increases survival and recombination efficiency through an as-yet unknown mechanism. Data shown are mean±standard deviation (n=3). FIG. 19D, lacZ genotypes deduced from FIG. 30B were confirmed by PCR and agarose gel electrophoresis, revealing either parental loci or recombination products containing integrated ISGst2. FIG. 19E, The transposon-mediated recombination stimulation involves the use of a designed insert that carries homology arms (shown in blue) flanking the integration site. The cargo for insertion is surrounded by IS200/IS605 transposon ends. The process starts by transforming cells with the insert, along with the TnpA transposase enzyme. Additionally, an enzyme that generates a double-strand break (DSB), such as Cas9, is used to target a specific site that matches the homology arms. This stimulates recombination, allowing the cargo to be inserted at the desired location.

FIGS. 20A-20D show the genomic architecture and endogenous splicing activity of TnpB-encoding IStrons. FIG. 20A, IS607-family transposons mobilize through a dsDNA intermediate using a serine-family recombinase (TnpA_{S}, right), in contrast to IS200/IS605-family transposons, which mobilize through a ssDNA intermediate using a tyrosine-family recombinase (TnpA_{Y}, left). Transposons of both families are bounded by conserved left end (LE) and right end (RE) sequences, encode tnpB accessory genes, excise as circular intermediates, and generate scarless donor joints that precisely regenerate the native genomic sequence. FIG. 20B, Genetic architecture of representative IS605 and IS607-family IS elements in comparison to closely related IStrons. Both families encode TnpA and TnpB proteins, but element ends are different: IStrons have a notably longer LE where they harbor catalytic core of the intron. FIG. 20C, Phylogenetic tree of group I introns that are structurally related to the CboIStron group I intron (left), with genetic architectures of select clades schematized (right). The outer rings of the tree indicate associations with TnpA_{S} or TnpA_{Y}, as well as whether the group I intron is encoded within an rRNA locus. The green and blue colors indicate associations with TnpB nucleases or homing endonucleases (HE), which fall into many distinct enzyme families (LAGLIDADG, GIY-YIG, HNH, His-Cys Box, and Endonuclease VII). Bootstrap values are indicated for major nodes. FIG. 20D, RNA-seq and whole-genome sequencing (WGS) data from two representative IS607-family transposons in C. senegalense, that encode identifiable group I introns; annotated genes are schematized below the graphs. RNA-seq coverage corresponding to putative ωRNAs are labeled, as are the number and connectivity of spliced exon-exon junction reads (orange). Quantitative comparison of exon-intron and exon-exon reads yields an apparent splicing percentage at the RNA level (RNA-seq graphs, top left), which was compared to similar junction reads at the DNA level (WGS graphs, top left). These analyses indicate the CseIStron-1 undergoes highly efficient splicing without any evidence of transposon excision, whereas the low-level apparent CseIStron-2 splicing (2%) can be explained by low-level transposon excision within the bacterial culture, as inferred by WGS analysis.

FIG. 21 shows the evolutionary and neighborhood analyses of TnpB, TnpA, and group I introns. (left) Unrooted phylogenetic tree of bacterial TnpB homologs in which cluster representatives are highlighted (green) that contain any member associated with a group I intron. Bootstrap values are indicated for major nodes. (right) Focused phylogenetic tree of TnpB homologs, including a much larger set of additional representatives from all clusters. Neighborhood analyses were performed on the genomic contexts of each tnpB gene, revealing associations with tnpA_{S} (IS607-family), tnpA_{Y} (IS200/IS605-family), group I introns (IStron), and ωRNA loci. Large groups of IS607-(blue sector) and IS200/IS605-family (red sector) IStrons were identified, and representative CdiIStron, CboIStron, and CseIStron members are annotated. Bootstrap values are indicated for major nodes.

FIGS. 22A-22F show the genomic and functional analysis of IS200/IS605-family IStrons from C. difficile (CdiIStron). FIG. 22A, transposon left end (LE; SEQ ID NO: 259) and right end (RE, SEQ ID NO: 260) covariance models for CdiIStron; the predicted LE and RE secondary structures recognized by TnpA_{Y} during transposon excision and integration are shown in the inset (top). A homologous C. difficile genomic locus lacking the transposon insertion is shown below. FIG. 22B, DNA multiple sequence alignment of transposon left end (LE, SEQ ID NOs: 261-270) and right end (RE, SEQ ID NOs: 271-280) sequences for 10 select CdiIStrons, based on comparative genomics and covariance models, with a consensus sequence shown at the top. The transposon adjacent motif (TAM), transposon encoded motif (TEM), and DNA guide sequences for both LE and RE are highlighted in yellow (TAM and LE guide) and orange (TEM and RE guide); dotted black lines indicate the upstream and downstream transposon boundaries. FIG. 22C, Secondary structure of the group I intron from a representative CdiIStron, with scaffold, substrate, and catalytic domains colored in green, brown, and yellow, respectively. Paired stem-loops defined as P1-P9, according to conventions defined by Hasselmayer et al. (Anaerobe. 2004 April; 10 (2): 85-92); the region that harbors tnpA_{y} and/or tnpB ORFs is indicated, as are the predicted 3′ and 5′ splice sites (SS). FIG. 22D, Schematic showing the predicted exon-exon junction products upon self-splicing of two representative CdiIStrons, compared to the coding sequences from otherwise isogenic strains that lack the IStron insertion. Protein sequences SEQ ID NOs: 281-285; DNA sequences SEQ ID NOs: 287-292, top to bottom respectively. FIG. 22E, Predicted ωRNA secondary structure (SEQ ID NO: 293) for a representative CdiIStron, based on secondary structure folding and alignment to the covariance model. The region also recognized by TnpA_{Y} at the DNA level is highlighted in orange. A cryoEM structure of DraTnpB (ISDra2) bound to its ωRNA substrate (PDB ID: 8BF8) is shown at right, highlighting the stem-loop (orange) that is recognized similarly at the RNA and DNA levels by TnpB and TnpA_{Y}, respectively. FIG. 22F, Secondary structure of the transposon RE SSDNA (SEQ ID NO: 294) for the same CdiIStron from FIG. 22E, based on covariance modeling; predicted DNA-DNA base-pairing between the DNA ‘guide’ (red) and transposon-encoded motif (TEM; orange) is highlighted. An X-ray crystal structure of HpyTnpAY bound to its RE substrate (PDB ID: 2A60) is shown at right, highlighting the stem-loop (orange) that is recognized similarly at the RNA and DNA levels by TnpB and TnpA_{Y}, respectively.

FIG. 23 shows comparative sequence and RNA-seq analyses of C. difficile intron and IStron elements. RNA-seq read coverage mapping to the indicated CdiIStron loci in C. difficile strain Cd1, as well as a representative standalone group I left (top left) and non-intron-containing IS605-family element (top right). The genomic coordinates are shown, as are gene annotations below each graph. RNA-seq coverage corresponding to the folded portion of group I introns are indicated. The number and connectivity of spliced exon-exon junction reads are highlighted, and quantitative comparison of exon-intron and exon-exon reads yields an apparent splicing percentage at the RNA level (top left of each graph). These analyses reveal a wide range of CdiIStron splicing efficiencies, though numbers may also be affected by DNA transposon loss within the population.

FIGS. 24A-24F show the genomic and functional analysis of IS607-family IStrons from C. botulinum (CboIStron). FIG. 24A, Schematic of episomal prophage in C. botulinum strain 1Cb16868 (NCBI accession ID: NZ_CM003334.1), highlighting the location of the botulinum neurotoxin gene and IS605-family, IS607-family elements, and IS607-family IStron elements. FIG. 24B, Transposon left end (LE) and right end (RE) definitions for two representative CboIStron elements, based on comparative genomics. Homologous C. botulinum genomic loci lacking the transposon insertions are shown below, which support the inferred transposon and splicing boundaries; the protein encoded by both IStron-interrupted genes are indicated. Genomic coordinates and NCBI genomic accession IDs are indicated at left, as are sequence identities between the sequences being compared (shaded wedges). FIG. 24C, DNA multiple sequence alignment of transposon LE (SEQ ID NOs: 295-304) and RE (SEQ ID NOS: 305-314) sequences for 10 select CboIStrons, with a consensus sequence shown at the top. The predicted transposon adjacent motif (TAM) is highlighted in yellow; dotted black lines indicate the upstream and downstream transposon boundaries. The top row corresponds to CboIStron-1 from FIG. 24B, which is the source of the TnpA_{S}, TnpB, ωRNA, and intron constructs used in heterologous E. coli experiments. FIG. 24D, Secondary structure of the group I intron from a representative CboIStron, with scaffold, substrate, and catalytic domains colored in green, brown, and yellow, respectively. Paired stem-loops defined as P1-P9, according to conventions defined by Hasselmayer et al. (2003); the region that harbors tnpA_{S} and/or tnpB ORFs is indicated, as are the predicted 3′ and 5′ splice sites (SS). FIG. 24E, Schematic showing the predicted exon-exon junction products upon self-splicing of two representative CboIStrons from FIG. 24A, compared to the coding sequences from otherwise isogenic strains that lack the IStron insertion. Protein sequences SEQ ID NOs: 315-320; DNA sequences SEQ ID NOs: 321-326, top to bottom respectively. FIG. 24F, Comparison of ωRNAs from well-studied representative IS605- and IS607-family transposons from D. radiodurans and Xylella fastidiosa (top), as well as IS607- and IS607-family CdiIStron and CboIStrons, respectively (bottom) (SEQ ID NOs: 327-330, respectively). Distinct RNA secondary structure motifs are labeled, alongside predicted pseudoknot (PK) interactions, and the guide sequence at the ωRNAs 3′ end is shown in blue. For IStrons, the guide sequence immediately follows the predicted 3′ splice site.

FIGS. 25A-25B show the evolutionary and neighborhood analyses of transposon-associated Arc-like proteins. FIG. 25A, Phylogenetic tree of Arc-like proteins, revealing genetic associations with TnpB (IS-family transposons) and Cas12k (CRISPR-associated transposons). FIG. 25B, Genetic architecture of representative transposable elements encoding Arc-like proteins (orange arrow), including IStron, IS, and CAST elements. Relevant genes are annotated, and putative transposon boundaries are indicated with inverted green triangles.

FIGS. 26A-26G show that CboTnpA_{S} catalyzes efficient IStron excision and integration, with unique dinucleotide requirements. FIG. 26A, Schematic of transposon excision assay using a CboTnpA_{S} expression plasmid (pTnpA_{S}) and CboIStron donor plasmid harboring a mini-transposon with LE and RE boundaries (pDonor). Expected substrates and products generated upon transposon excision by PCR are indicated, as are the primer binding sites. FIG. 26B, Gel electrophoresis (left) and Sanger sequencing (right) of PCR products (SEQ ID NOs: 331-333) from FIG. 26A, demonstrating that TnpA_{S} is active in recognizing and excising the IStron. Cell lysates were tested after overnight expression of TnpA_{S} with the indicated substrates, which included an IStron mutant containing mismatched dinucleotides (LE: 5′-GG-3′, RE: 5′-TT-3′), and IStrons with RE or LE deletions. Marker denotes a positive excision control, and U and E refer to unexcised and excised products. M denotes a S67A TnpA_{S} mutant. Sanger sequencing is shown at right, with the rejoined TAM and putative ωRNA-matching target highlighted in yellow and orange, respectively. FIG. 26C, Quantitative PCR-based assay to determine the minimal left end (LE) and right end (RE) sequences necessary for efficient IStron excision. Serial truncations were tested, starting with a WT substrate containing 581 bp and 221 bp derived from the native LE and RE, respectively. FIG. 26D, Schematic of transposon integration assay using a TnpA_{S} expression plasmid (pTnpA_{S}) and IStron circularized intermediate donor plasmid harboring abutted LE and RE sequences (pDonor_{CI}). With this suicide vector that cannot propagate in a pir-strain, transposon integration events can be enriched using chloramphenicol selection and deep-sequenced using TagTn-seq. FIG. 26E, Cell viability data from experiments in FIG. 26D, plotting as colony forming units (CFU), when cells contained either mutant S67A (M) or WT TnpA_{S}. FIG. 26F, Genome-wide distribution of TagTn-seq reads from experiments in FIG. 26D using WT TnpA_{S}, mapped to the E. coli genome. Data are shown for pDonor_{CI} substrates containing either a GG (top) or GC (bottom) dinucleotide. FIG. 26G, Meta-analyses of target site preferences and integration product dinucleotides at the LE and RE junction, for the genome-wide insertion data with GG and GC dinucleotide substrates shown in FIG. 26F; the number of unique integration sites is indicated. The preferred genomic target motif is GG for both substrates, but high-throughput sequencing across the LE and RE junction for integration products clearly reveals that non-canonical dinucleotides in pDonor_{CI} template correspond to non-canonical dinucleotides at the LE junction upon recombinational integration.

FIGS. 27A-27D show molecular and sequence determinants of CboIStron DNA excision by CboTnpA_{S}. FIG. 27A, Schematic of transposon excision assay using a CboTnpA_{S} expression plasmid (pTnpA_{S}) and CboIStron donor plasmid harboring a mini-transposon with LE and RE boundaries (pDonor). Expected substrates and products generated upon transposon excision are indicated, as are the primer binding sites for quantitative excision measurements using qPCR. FIG. 27B, Gel electrophoresis (left) and Sanger sequencing (right) of PCR products (SEQ ID NOs: 334-336, respectively) from FIG. 27A, demonstrating the cellular presence of transposon circular intermediates (CircInt) in a TnpA_{S}-dependent reaction. Primers were designed to amplify across the joined LE and RE, such that only the indicated product (top) would yield a PCR amplicon 280 bp in size. Reactions were performed in biological duplicates and contained either empty vector (−), mutant S67A TnpA_{S} (M), or WT TnpA_{S} (+). The Sanger sequencing data demonstrate that amplicons contained the inverted RE-LE junction, with recombined GG core dinucleotide. FIG. 27C, Gel electrophoresis of PCR products from experiments performed as in FIG. 27A using mini-Tn substrates that contained serial truncations of either the LE (top) or right end (bottom). These experiments indicate that only 40-bp and 60-bp are necessary on the LE and RE, respectively, for WT efficiencies of excision. The length of the truncated end is shown above each lane, counting from the first bp inside the LE or RE, and the mobility of PCR products represent the unexcised (U) or excised (E) products. Note that the excised product is the same size in all cases, as expected. Control lanes on the far right lacked TnpA_{S} and thus were inactive for excision. FIG. 27D, Schematic of minimal transposon design containing 60-bp LE and RE sequences (SEQ ID NO: 337 and 338, respectively) (top), sequence of minimal ends, highlighting the identification of putative TnpA_{S} binding site (yellow highlights), and mini-Tn DNA excision assay measured by qPCR. Binding sites were mutated independently or in tandem, across either the entire motif or only the TATA portion, as indicated. In all cases, disruption of two or more motifs completely abolished detectable DNA integration.

FIGS. 28A-28F show detailed investigation of target specificity and synergistic TnpA_{S}-TnpB activity during transposon integration and recombination. FIG. 28A, TagTn-seq workflow for deep sequencing of genome-wide transposition events in E. coli using TnpA_{S} and circularized intermediate donor molecules (pDonor_{CI}). Transposon-containing molecules are selectively amplified in a nested PCR after tagmentation of high-molecular weight genomic DNA, followed by next-generation sequencing (NGS), computational filtering, and read mapping back to the E. coli reference genome (left). Meta-analysis of the genomic coordinates containing transposon insertions enables identification of conserved target-site motifs (right). FIG. 28B, Genome-wide distribution of TagTn-seq reads from experiments as in FIG. 28A using WT TnpA_{S} and pDonor_{CI} containing a core GG dinucleotide, mapped to the E. coli genome (bottom). Meta-analyses reveal a strict GG dinucleotide requirement at the site of transposon integration (top). FIG. 28C, Experiments in FIG. 28B were repeated, but using pDonor_{CI} substrates containing non-canonical core dinucleotides, as indicated. Analysis of the resulting integration sites revealed that integration site preference could only partially be reprogrammed with altered core dinucleotides; that the nucleotide sequence in the LE insertion product always matched the altered core dinucleotide on pDonor_{CI}; but that a G was preferentially installed at the +1 position in the RE insertion product, regardless of altered core dinucleotide on pDonor_{CI}. FIG. 28D, PCR and gel analysis of lacZ genotypes, demonstrating the role of TnpB in promoting transposon retention by reducing the relative frequency of excision products (E) relative to unexcised transposon substrates (U). Cells expressed either wild-type (+) or mutant (S67A) TnpA (M), in the presence of WT (+) or nuclease-dead (D189A) dTnpB (d). FIG. 28E, Workflow to measure transposon recombination in E. coli with TnpA_{S} and TnpB. Native CboIStron transposons with TnpA_{S} or either WT or nuclease-dead dTnpB were inserted in the reverse direction at a compatible TAM in plasmid-encoded lacZ, such that splicing could not generate a lacZ+ phenotype. Plasmids were used to transform E. coli cells harboring a wild-type lacZ locus. RNA-guided DNA cleavage of genomic lacZ triggers recombination with the ectopic CboIStron-lacZ, leading to white colonies. Tet, tetracycline. FIG. 28F, Bar graph shows the plasmid transformation efficiency for each condition, with white bars reporting colonies with a lacZ phenotype; E, empty vector. The data reveal that TnpA_{S} and TnpB co-operate for efficient self-mobilization into a vacant donor site via recombination, but only in the presence of nuclease-active TnpB. Data are mean±s.d. (n=3).

FIGS. 29A-29I show ChoTnpB is a potent RNA-guided nuclease that prevents CboTnpA_{S}-mediated transposon extinction. FIG. 29A, Schematic of RIP-seq workflow to uncover RNA binding partners of ChoTnpB using the pEffector shown. FIG. 29B, RIP-seq read coverage for experiments with WT TnpB and RuvC-inactivated dTnpB (D189A) mapped to pEffector (left). The pre-ωRNA processing site is indicated with a red triangle, in both the graph and the RNA schematic shown at the right. The green region labeled “tnpB” corresponds to the 3′ end of the ORF. FIG. 29C, Schematic showing the regenerated target site that is produced upon transposon excision, with abutted TAM and target site. FIG. 29D, Bacterial spot assays demonstrate that TnpB is highly active for RNA-guided DNA cleavage of the donor joint, as assessed by plasmid interference assays. TnpB was expressed with either a targeting (T) or non-targeting (NT) ωRNA from a native IStron or synthetic expression plasmid context, and transformants were serially diluted, plated on selective media, and cultured at 37° C. for 24 h. Additional controls included a mutant TAM (“-”, 5′-ACCC-3′) or RuvC-inactive (D189A) dTnpB. FIG. 29E, Schematic indicating the uncertainty over whether nucleotides within the ωRNA scaffold might influence TAM specificity through direct base-pairing, especially since TnpA_{S} could theoretically recognize either of two adjacent GG core dinucleotides defining the transposon boundary. Target SEQ ID NOs: 339 and 340; guide RNA SEQ ID NO: 341. FIG. 29F, Results from a TAM library cleavage assay using a wild-type ωRNA, revealing that ChoTnpB requires a consensus 5′-(T)GGG-3′ TAM for efficient DNA cleavage. The WebLogo was generated using the 20-most depleted sequences after deep sequencing pTarget from surviving colonies (see FIG. 11A). FIG. 29G, Violin plots of TAM enrichment from TAM library assays using variant TnpB-ωRNA expression plasmids with the indicated nucleotide in the −1 position of the ωRNA. Data are plotted as the log 2-fold enrichment relative to the input library, with specific members highlighted; dotted line represents 5-fold depletion. All ωRNA variants depleted only 5′-TGGG-3′ TAMs, indicating an absence of base-pairing at the −1 position. FIG. 29H, Schematic of assay to measure transposon fate in E. coli with TnpA_{S} and TnpB-ωRNA, and bar graph FIG. 29I showing the frequency of transposon excision/retention for each condition, quantified by blue/white colony screening. A mini-Tn was inserted at a compatible TAM in lacZ, and cells were transformed with plasmids expressing wild-type or mutant S67A TnpA (M) and/or TnpB or dTnpB. White or blue colonies indicate transposon retention or excision, respectively. Data are mean±s.d. (n=3); E, empty vector; ND, not detected.

FIGS. 30A-30D show library experiments to determine TAM specificity by ChoTnpB. FIG. 30A, Schematic of TAM library cleavage assay, in which a plasmid expressing nuclease-active ChoTnpB and an associated ωRNA from within the native CboIStron (pEffector) is designed to cleave a target sequence flanked by randomized 6-mer (pTarget). Plasmid cleavage results in plasmid elimination, loss of cell viability, and depletion of the particular TAM upon library sequencing. FIG. 30B, WT (top) and non-canonical ωRNA variants screened in the TAM library assay, to investigate if base-pairing occurs at the −1 position in the ωRNA. NTS, non-target strand; TS, target strand. ωRNA stand sequences SEQ ID NOs: 342-345, top to bottom. FIG. 30C, Sequence WebLogo of top depleted library members for the ωRNA variants shown in panel FIG. 30B; The number of library members used to construct the weblogo is shown in the top left corner. Data for the WT ωRNA are replotted from is the same as shown in FIG. 29F. FIG. 30D, TAM wheels for the same ωRNA variants shown in FIG. 30B, generated using the 5% most depleted library members. These results indicate that the −1 position of the ωRNA does not confer any specificity in the recognized TAM motif.

FIGS. 31A-31D show CseIStrons encode functional self-splicing ribozymes that regenerate transposon-free transcripts. FIG. 31A, Schematic of general IStron splicing mechanism and E. coli-based cellular splicing assay. Exogenous GTP binding by the folded group I intron leads to a transesterification reaction at the 5′ splice site (SS), followed by attack of the 3′ SS by exon 1 to yield the ligated exon-exon product and excised intron. Spliced/unspliced products are detected and/or quantified by RT-PCR and RT-qPCR, respectively, using the primer pair strategies indicated at the bottom. FIG. 31B, Agarose gel electrophoresis of RT-PCR products from splicing assays in FIG. 31A with the indicated constructs, which shows the extent of unspliced (U) and spliced(S) products (top) relative to reference amplicons for a SpecR drug marker (middle) and exon1-LE junction (bottom). RT, reverse-transcriptase; Marker denotes a positive excision control; IStron (cat. mut.) contains a P7-P9 loop deletion in the intron catalytic core; IStron (TAM mut.) contains 5′-TGTA-3′ in the TAM and thereby disrupts base-pairing required for 5′ SS recognition. FIG. 31C, Sanger sequencing of RT-PCR products from FIG. 31B, for both the unspliced exon-intron boundaries (SEQ ID NOs: 346 and 347) (top) and the spliced exon-exon product (SEQ ID NO: 348) (bottom). These sequences are identical to the nucleotide sequences of unexcised and excised DNA sequences in FIG. 26. FIG. 31D, Quantitative measurements of the spliced/unspliced ratio by RT-qPCR using the assay in FIG. 31A, for the indicated constructs that contain variable ‘cargo’ sequences. The minimal construct harbors the CboIStron sequence after removal of tnpA_{S} and tnpB ORFs and exhibits a splicing ratio of ˜0.4, whereas splicing becomes nearly undetectable with cargos comprising either the tnpB gene (encoding functional TnpB), dtnpB, or a tnpB with in-frame stop codon ((*) dtnpB), all of which are ˜1180 bp in length. Constructs with alternative cargos containing the indicated length of unrelated lacZ sequence exhibited decreased splicing efficiency with increasing size, though at levels above that observed with tnpB, suggesting a potential role for the TnpB protein in splicing control.

FIGS. 32A-32C show detection and quantification of splicing and RNA-guided DNA cleavage activity. FIG. 32A, Templates for in vitro transcription (IVT)-based group I intron splicing assays were generated by PCR, and lacked any detectable truncation products (left). The ensuing IVT reactions immediately revealed evidence of spliced exon-exon junction products, as detected by RT-qPCR (right), which matched the expected size based on a Marker control; IStron (cat. mut.) contains a P7-P9 loop deletion in the intron catalytic core. U, unspliced; S, spliced. FIG. 32B, Bacterial spot assays demonstrate that TnpB is equally active for RNA-guided DNA cleavage when the ωRNA is expressed in trans from a separate ωRNA expression plasmid. The in trans activity was equivalent whether or not the mini-Tn also encoded the full-length group I intron (gI). Transformants were serially diluted, plated on selective media, and cultured at 37° C. for 24 h. FIG. 32C, Comparison of simulated spliced/unspliced ratios, generated by mixing mock-spliced and mock-unspliced lysates in known ratios, versus experimentally determined spliced-unspliced ratios measured by RT-qPCR, using the strategy described in FIG. 31A. The results demonstrate the accuracy of our quantification method.

FIGS. 33A-33H show competition between intron splicing and TnpB-ωRNA activity establishes a balance between transposon stealth and preservation. FIG. 33A, Schematic of CboIStron ωRNA secondary structure encoded within the transposon RE, with stem-loops (SL), truncation coordinates, and pseudoknot (PK) motifs labeled. FIG. 33B, RT-qPCR analysis of splicing efficiency for IStron variants in which the RE/ωRNA region was systematically truncated relative to the full-length construct (221 bp). The large splicing change with the 180-bp construct suggests sequence and/or structural features around this position that repress splicing in the full-length design. FIG. 33C, Bacterial spot assays for the same RE/ωRNA deletion constructs in FIG. 33B, in which RNA-guided DNA cleavage leads to cell death. TnpB was expressed with either a targeting (T) or non-targeting (NT) ωRNA, and transformants were serially diluted, plated on selective media, and cultured at 37° C. for 24 h. Any deletion beyond 180 bp eliminates DNA cleavage activity. FIG. 33D, RT-qPCR analysis of splicing efficiency (left), and spot assays to monitor RNA-guided DNA cleavage activity (right), for the indicated RE/ωRNA pseudo-knot mutations, plotted as in FIGS. 33B and 33C. PK_{MUT1} and PK_{MUT2} contain mutations to either the upstream or downstream motif, whereas PK_{COMP} contains compensatory mutations in both motifs. The results indicate that ωRNA PK disruption abrogates TnpB-mediated DNA cleavage, while any mutation to the downstream PK motif abrogates intron splicing; intron splicing is strongly stimulated by mutations to the upstream PK motif. FIG. 33E, RT-qPCR analysis of splicing efficiency in the presence of a second effector plasmid harboring tnpB, dtnpB, or a codon-optimized (CO) dinpB gene. Empty refers to an empty vector control. These results reveal a repressive role of TnpB in intron splicing. FIG. 33F, RT-qPCR analysis of splicing efficiency in the absence or presence of TnpB, for the indicated RE/ωRNA variants. The repressive effect of TnpB on splicing is largely ablated when the ωRNA scaffold is missing (20-bp RE) or replaced with an unrelated sequence (Insert₁+20-bp RE). FIG. 33G, RT-qPCR analysis of splicing efficiency for the full-length (221-bp) or truncated 20-bp RE variant, without (“-”) or with three distinct sequence insertions replacing the ωRNA scaffold. These experiments demonstrate that the native ωRNA scaffold sequence alone acts as a potent repressor of splicing efficiency. FIG. 33H, Overall model for the balanced effects of intron splicing, TnpB-ωRNA, and TnpA_{S} transposition activity in the maintenance and spread of IS607-family IStron elements. Similarly to IS200/IS605-family transposons, scarless DNA excision by TnpA_{S} for IS607-family elements leads to transposon loss at the donor site and thus eventual transposon extinction, without the crucial function provided by TnpB-ωRNA in generating targeted DNA double-strand breaks and triggering homologous recombination to maintain presence of the transposon (top). Unlike canonical IS200/IS605 and IS607-family transposons, group I intron-containing IStrons mitigate their fitness costs on the host by splicing themselves out of interrupted transcripts at the RNA level, thereby restoring functional gene expression (bottom, middle). Splicing and ωRNA maturation are mutually exclusive, since splicing severs the ωRNA scaffold and guide sequences, and TnpB represses splicing through competitive binding of the 3′ SS. The competition between intron splicing and TnpB-ωRNA activity thus serves to regulate the dual objectives of maintaining transposon stealth and promoting transposon proliferation for IStron elements. A similar mechanism is hypothesized for IS200/IS605-family IStrons.

FIGS. 34A-34E show structure and sequence determinants of intron splicing and RNA-guided DNA cleavage. FIG. 34A, Agarose gel electrophoresis of RT-PCR products from splicing assays with the indicated serial deletions in the transposon left end/intron region (LE/intron, left) or transposon right end/ωRNA region (RE/ωRNA, right). Unspliced (U) and spliced(S) products are indicated, relative to reference amplicons for a SpecR drug marker (bottom). Any deletion in the 581-bp LE/intron region eliminates splicing, whereas deletions of everything but the terminal 20 bp in the RE/ωRNA region are tolerated. NTC, non-template control. FIG. 34B, Quantitative measurements of the spliced/unspliced ratio by RT-qPCR for the indicated constructs that harbor deletions in the RE/ωRNA region. The WT construct contains 221-bp of the RE, whereas a shorter 20-bp construct exhibits far greater splicing activity. Any deletion beyond 16 bp leads to a loss of splicing activity. FIG. 34C, Quantitative measurements of the spliced/unspliced ratio by RT-qPCR for the indicated constructs that harbor stem-loop (SL) deletions RE/ωRNA region, as defined in FIG. 33A. The WT constructs contains 221-bp of the RE. FIG. 34D, Bacterial spot assays for the same RE/ωRNA SL deletion constructs in FIG. 34C, in which RNA-guided DNA cleavage leads to cell death. TnpB was expressed with either a targeting (T) or non-targeting (NT) ωRNA, and transformants were serially diluted, plated on selective media, and cultured at 37° C. for 24 h. Deletion of any SL except SL4 completely abolished DNA cleavage activity. FIG. 34E, Quantitative measurements of the spliced/unspliced ratio by RT-qPCR for an intron substrate driven by the indicated variable-strength promoters, with (yellow) or without (green) TnpB co-expression. The repressive effect of TnpB is strongest at low expression levels. “-” refers to no specific promoter inserted before the intron containing gene.

### DETAILED DESCRIPTION

The disclosed systems, kits, and methods provide systems and methods for nucleic acid modification.

Insertion sequences (IS) are compact and pervasive transposable elements found in bacteria, which encode the genes for their mobilization and maintenance. IS200/IS605 elements undergo ‘peel-and paste’ transposition catalyzed by the TnpA transposase, but intriguingly, they also encode diverse, TnpB-family nucleases that are evolutionarily related to the CRISPR-associated effectors Cas9 and Cas12. Although recent studies demonstrated that TnpB-family proteins function as an RNA-guided DNA endonucleases, the broader biological role of this activity has remained enigmatic.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence of the present disclosure after aligning the two sequences and introducing gaps, if necessary, to achieve the maximum percent identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46:453 (1960) and Doty et al., Proc. Natl. Acad. Sci. USA, 46:461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) may also be considered a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Systems

Transposons encode RNA-guided DNA nucleases that are evolutionary ancestors to CRISPR-Cas9 and Cas12 enzymes, named IscB and TnpB respectively, but are roughly four times smaller and compact in size. These smaller nucleases function (e.g., in human cells) for targeted DSBs and genome editing. Because of their smaller size, IscB and TnpB nucleases offer promise for next-generation genome editing, since they are within the size range where packaging inside of small viral vectors (like AAV) becomes feasible, for example for use in base editing, prime editing, and epigenome editing. Indeed, IscB and TnpB show promise for a similar range of diverse genome engineering applications as has already been demonstrated with Cas9 and Cas12, but again, using a smaller and more compact protein-RNA system.

Provided herein are systems for modifying a target nucleic acid that include TnpA, TnpB, and/or IscB, or one or more nucleic acids encoding thereof. In some embodiments, the systems comprise: a TnpA protein, a TnpB protein, an IscB protein, or a combination thereof, and/or one or more nucleic acids encoding thereof; and optionally, at least one guide RNA, or one or more nucleic acids encoding thereof, complementary to at least a portion of a target nucleic acid.

In some embodiments, the system comprises, consists of, or consists essentially of a TnpA protein. In some embodiments, the system comprises, consists of, or consists essentially of a TnpA protein and at least one guide RNA.

In some embodiments, the system comprises, consists of, or consists essentially of a TnpB protein. In some embodiments, the system comprises, consists of, or consists essentially of a TnpB protein and at least one guide RNA.

In some embodiments, the system comprises a TnpA protein and a DNA nuclease capable of inducing site-specific single or double strand breaks, or one or more nucleic acids encoding thereof. The Cas CRISPR/Cas nuclease can be from any Type or Class of CRISPR-Cas systems (e.g., Class 1, Class 3, Types I-VI, or any of subtypes thereof). In some embodiments, the CRISPR/Cas nuclease is Cas9 or Cas12.

In some embodiments, the DNA nuclease is an RNA-guided DNA nuclease encoded by insertion sequences. In some embodiments, the DNA nuclease encoded by insertion sequences is IscB, IsrB, TnpB, or Fanzor.

In some embodiments, the DNA nuclease is a homing endonuclease. In some embodiments, the homing endonuclease is ISce-I, ICre-I, or HO.

In some embodiments, at least one of the TnpA, TnpB, and IscB proteins is derived from Geobacillus stearothermophilus, Clostridium botulinum, Clostridium senegalense or Clostridioides difficile.

The TnpA protein may be a serine-family recombinase or, alternatively, a tyrosine-family recombinase. In some embodiments, the TnpA protein comprises an amino acid sequence having at least 70% (e.g., having at least 75%, at least 80%, at least 85%, at least 90%, at least 92%, at least 95%, at least 96%, at least 97%, at least 98%, or at least 99%) identity to any of SEQ ID NO: 11, 21, 25, and 38-41. In some embodiments, the TnpA protein comprises an amino acid sequence of any of SEQ ID NO: 11, 21, 25, and 38-41.

The TnpB protein may be derived from an IS607-family or an IS200/IS605-family. In some embodiments, the TnpB protein comprises an amino acid sequence having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-4, 6-9, 17, 22-24, 30-37, and 42-50. In some embodiments, the TnpB protein comprises an amino acid sequence of any of SEQ ID NO: 1-4, 6-9, 17, 22-24, 30-37, and 42-50.

In some embodiments, the IscB protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 5 or 10. In some embodiments, the IscB protein comprises an amino acid sequence of SEQ ID NO: 5 or 10.

The TnpA protein may be a serine-family recombinase or a tyrosine-family recombinase. TnpA derived from IS607-family transposons represents a serine-family recombinase, hereby indicated by the suffix “(S)” to signify its serine catalytic active site. Contrarily, G. stearothermophilus TnpA corresponds to a tyrosine-family recombinase, referenced as TnpA (Y), emphasizing its tyrosine catalytic active site.

Any of the proteins described or referenced herein may comprise one or more amino acid substitutions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

For example, the TnpA, TnpB, and or IscB protein may be fully or partially catalytically inactivated by one or more amino acid substitutions. For example, D196A GstTnpB2, D58A/H209A/H210A IscB, D189A Cho TnpB, and others as described herein. Fully or partially catalytically inactivated variants of the proteins as disclosed herein may still function as a nucleic acid binding protein, alone or in coordination with a guide RNA or other protein, with the targeting capabilities of the fully functioning protein.

Any of the proteins disclosed herein may further comprise one or more proteins, polypeptides (e.g., protein domain sequences), or peptides fused to the polypeptide. For example, the proteins disclosed herein may be fused to another protein or protein domain that provides for tagging or visualization (e.g., GFP). The one or more proteins, polypeptides (e.g., protein domain sequences), or peptides may be appended at an N-terminus, a C-terminus, internally, or a combination thereof. The one or more proteins, polypeptides (e.g., protein domain sequences), or peptides may be fused in any orientation in relationship to the disclosed protein.

Any of the proteins described or referenced herein may be linked to an effector polypeptide. Effector polypeptides include proteins or protein domains that have additional functionality or activity useful to target to certain DNA sequences. The effector polypeptide. may comprise a number of functionalities, including but not limited to, nuclease function, recombinase function, epigenetic modifying function, transposase function, integrase function, resolvase function, invertase function, protease function, DNA methyltransferase function, DNA demethylase function, histone acetylase function, histone deacetylase function, transcriptional repressor function, transcriptional activator function, DNA binding protein function, transcription factor recruiting protein function, nuclear-localization signal function, DNA editing function (e.g., deaminase) or any combination thereof. For example, some effector domains function in transcriptional regulation via their ability to interact with the basal transcriptional machinery and general co-activators, interact with other transcription factors to allow cooperative binding, and/or directly or indirectly recruit histone and chromatin modifying enzymes.

In some embodiments, the system described herein is used to modulate gene regulatory activity, such as transcriptional or translational activity. For example, the at least one effector polypeptide may comprise activator and/or repressor activity that can affect transcription upstream and downstream of coding regions, and can be used to activate or repress gene expression. In some embodiments, the at least one effector polypeptide may include domains from transcription factors (activators, repressors, coactivators, co-repressors), silencers, and/or chromatin associated proteins and their modifiers (e.g., methylases, demethylases, acetylases and deacetylases).

Accordingly, in some embodiments, a system as disclosed herein having a transcription activator effector polypeptide can be used to directly increase gene expression. In some embodiments, a system as disclosed herein comprising a transcriptional protein recruiting domain, or active fragment thereof, can be used to recruit transcriptional activators or repressors to a specific nucleic acid sequence to localize activators and repressors to modulate gene expression in a targeted manner.

In some embodiments, the effector polypeptide comprises transcriptional repressor function. Transcription repressors prevent, partially or completely, the transcription of genes near to their target site. Exemplary transcriptional repressors include, but are not limited to, KRAB-domain containing proteins, SID, and Sp1.

In some embodiments, the effector polypeptide comprises transcriptional activator function. Transcriptional activators can be generally defined as proteins, or domains thereof, that bind to specific sites on promoter DNA and bring about increased transcription of specific genes through interactions with other proteins. Exemplary transcriptional activators include, but are not limited to, VP64, p65, p53, c-Myb, GATA-1, EKLF, MyoD, E2F, dTCF, Tat, HSF1, RTA and SET7/9.

In some embodiments, the effector polypeptide comprises DNA methyltransferase or DNA methylase function. DNA methyltransferases (DNMT's) are a family of DNA modifying proteins composed of different isomers (e.g., DNMT1, DNMT3A, and DNMT3B). Other exemplary DNA methyltransferases include SssI methylase, AluI methylase, HaeIII methylase, HhaI methylase, and HpaII methylase. Their main mechanism of action is addition of a methyl group to the fifth carbon of a cytosine residue (5mc) located adjacent to a guanine residue.

In some embodiments, the effector polypeptide comprises DNA demethylase function. DNA demethylation can be mediated by at least three enzyme families: (i) the ten-eleven translocation (TET) family, mediating the conversion of 5mC into 5hmC; (ii) the AID/APOBEC family, acting as mediators of 5mC or 5hmC deamination; and (iii) the BER (base excision repair) glycosylase family involved in DNA repair.

Kinases, phosphatases, and other proteins that modify or regulate other polypeptides involved in gene regulation are also useful as effector polypeptides. Such modifiers are often involved in switching on or off transcription mediated by, for example, hormones. Other useful domains for regulating gene expression can also be obtained from the gene products of oncogenes (e.g., myc, jun, fos, myb, max, mad, rel, cts, bcl, myb, mos family members) and their associated factors and modifiers.

The effector polypeptide can be used to target enzymatic activity to locations containing the target nucleic acid sequence to which the gRNA is directed. For example, in some embodiments, effector polypeptides having integrase or transposase activity can be used to promote integration of exogenous nucleic acid sequence into specific nucleic acid sequence regions and/or eliminate (knock-out) specific endogenous nucleic acid sequence.

Integrases allow for the insertion of nucleic acids, for example, into a host genome (mammalian, human, mouse, rat, monkey, frog, fish, plant (including crop plants and experimental plants like Arabidopsis), laboratory or biomedical cell lines or primary cell cultures, C. elegans, fly (Drosophila), etc.). Integrases are found in a retrovirus such as HIV (human immunodeficiency virus) and lambda integrase.

In some embodiments, the effector polypeptide comprises transposase functionality. Transposases are enzymes that bind to the end of a transposon and catalyze its movement by a cut and paste mechanism or a replicative transposition mechanism. Exemplary transpoases include, but are not limited to, Tc1 transposase, Mos1 transposase, Tn5 transposase, and Mu transposase

In some embodiments, the effector polypeptide modifies epigenetic signals and thereby modifies gene regulation, for example by promoting histone acetylase and histone deacetylase activity. The term “epigenetic modifier,” as used herein, refers to a protein or catalytic domain thereof having enzymatic activity that results in the epigenetic modification of DNA, for example, chromosomal DNA. Epigenetic modifications include, but are not limited to, histone modifications including methylation and demethylation (e.g., mono-, di- and tri-methylation), histone acetylation and deacetylation, as well as histone ubiquitylation, phosphorylation, and sumoylation.

Histone acetylation and deacetylation are the processes by which the lysine residues within the N-terminal tail protruding from the histone core of the nucleosome are acetylated and deacetylated as part of gene regulation. These reactions are typically catalyzed by enzymes with histone acetyltransferase (HAT) or histone deacetylase (HDAC) activity. Histone acetyltransferases include GNAT family proteins (e.g., Gcn5, Gcn5L, p300/CREB-binding protein associated factor (PCAF), Elp3, HPA2 and HAT1) and MYST family proteins (e.g., Sas3, essential SAS-related acetyltransferase (Esa1), Sas2, Tip60, MOF, MOZ, MORF, and HBO1). Histone deacetylases fall into four classes. Class I includes HDACs 1, 2, 3, and 8. Class II is divided into two subgroups, Class IIA and Class IIB. Class IIA includes HDACs 4, 5, 7, and 9 while Class IIB includes HDACs 6 and 10. Class III contains the Sirtuins and Class IV contains only HDAC11. Classes of HDAC proteins are divided and grouped together based on the comparison to the sequence homologies of Rpd3, Hos1 and Hos2 for Class I HDACs, HDA1 and Hos3 for the Class II HDACs and the sirtuins for Class III HDACs.

The site-specific methylation and demethylation of histone residues are catalyzed by methyltransferases and demethylases, respectively. Histone methylases transfer methyl groups to amino acids (e.g., lysine and arginine) of histone proteins, ultimately effecting transcription of genes. Methylases include SET1, MLL, SMYD3, G9a, GLP, EZH2, and SETDB1. Histone demethylases catalyze the removal of methyl marks from histones, an activity associated with transcriptional regulation and DNA damage repair. Demethylases include, for example, KDM1A, KDM1B, KDM2A, KDM2B, UTX, UTY, Jumonji C (JmJC) domain-containing demethylases, and GSK-J4.

In some embodiments, the effector polypeptide comprises nuclease activity. A nuclease is an agent that induces a break in a nucleic acid sequence, e.g., a single or a double strand break in a double-stranded DNA sequence. Nucleases include those which cut at or near a preselected or specific sequence and those which are not site specific. For example, nucleases include, but are not limited to, zinc finger nucleases (ZFN), homing endonucleases, meganucleases, restriction enzymes, TAL effector nucleases, Argonaute nucleases, CRISPR nucleases, comprising, for example, Cas9, Cpf1, Csm1, CasX or CasY nucleases, micrococcal nuclease, staphylococcal nuclease, DNase I, T7 endonuclease, or catalytically active fragments thereof.

In some embodiments, the effector polypeptide comprises invertase activity. Invertase activity can be used to alter genome structure by swapping the orientation of a DNA fragment.

In some embodiments, the effector polypeptide comprises recombinase activity. A recombinase is a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3 (also known as TnpR), β-six, CinH, ParA, γδ, Bxb1, ϕC31, TP901, TG1, ϕBT1, R4, ϕRV1, ϕFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange.

In some embodiments, the effector polypeptide comprises resolvase activity. Resolvases are site-specific recombinases that function to excise (as a circle) a segment of DNA contained between two recombination sites (called res) and include, for example, Ruv C resolvase, Holiday junction resolvase Hjc, Tn3 and γδ resolvase.

In some embodiments, the effector polypeptide comprises a peptide or polypeptide sequence responsive to a ligand, such as a hormone receptor ligand binding domain, including, for example, the ligand binding domains of the estrogen receptor, the glucocorticosteroid receptor, and the like. Such effector domains can be used to act as “gene switches,” and be regulated by inducers, such as small molecule or protein ligands, specific for the ligand binding domain.

In some embodiments, the effector polypeptide comprises sequences or domains of polypeptides that mediate direct or indirect protein-protein interactions, including, for example, a leucine zipper domain, a STAT protein N terminal domain, and/or an FK506 binding protein.

In some embodiments, the effector polypeptide comprises DNA editing function (e.g., deaminase, DNA repair activity, DNA damage activity, dismutase activity, alkylation activity, depurination activity, oxidation activity, pyrimidine dimer forming activity, polymerase activity (e.g., reverse transcriptase), ligase activity, helicase activity, photolyase activity or glycosylase activity).

In some embodiments, the effector polypeptide comprises a deaminase, or functional fragment thereof. The deaminase, or functional fragment thereof may be derived from a naturally occurring deaminase or variant thereof (e.g., a protein, enzyme, or domain with an amino acid sequence having at least 70% identity to a naturally occurring deaminase). Alternatively, the deaminase may be a synthetic or engineered deaminase. In some embodiments, the deaminase, or functional fragment thereof, is an adenosine deaminase, also sometimes referred to as an adenine deaminase. In some embodiments, the adenosine deaminase is derived from a bacterium, such as, E. coli. In some embodiments, the deaminase, or functional fragment thereof, is a cytidine deaminase.

In some embodiments, the activity mediated by the effector polypeptide is a non-biological activity, such as a fluorescence activity (e.g., fluorescent proteins), luminescence activity (e.g., a luminescent protein or enzyme which results in luminescence when interacting with a substrate (e.g., luciferase)), or binding activity, such as those mediated by maltose binding protein (“MBP”), glutathione S transferase (GST), hexahistidine, c-myc, and the FLAG epitope, for facilitating detection, purification, monitoring expression, and/or monitoring cellular and subcellular localization of the polypeptide to which the effector domain is appended. In such embodiments, the systems can also be used as a diagnostic reagent, for example, to detect mutations in gene sequences, to purify restriction fragments from a solution, or to visualize DNA fragments of a gel.

The effector polypeptides described herein are illustrative and merely provide the skilled artisan with examples of effectors that can be used in combination with the systems and methods described herein.

In some embodiments, the effector polypeptide comprises a transcription activator, a transcription repressor, a base editor, an epigenetic modifier, a chromosomal locus imaging agent (e.g., fluorescent protein or protein tag), or a combination thereof.

In some embodiments, the effector polypeptide comprise fragments of proteins that have been separated from their natural DNA binding domains and engineered to be part of a fusion protein with the protein described herein. In some embodiments, the effector polypeptides are proteins which normally bind to other proteins or factors which result in their recruitment to a specific or non-specific nucleic acid.

Any of the proteins described or referenced herein may further have a nuclear localization sequence (NLS). The at least one nuclear localization sequence may be appended to the N-terminus, the C-terminus, or embedded in the protein (e.g., inserted internally within the open reading frame (ORF)). The polypeptides may comprise one or more nuclear localization sequences. The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprises a single cluster of positively charged or basic amino acids. In some embodiments, the monopartite NLS comprises a sequence of K-K/R-X-K/R, wherein X can be any amino acid. Exemplary monopartite NLSs include, without limitation, those from the SV40 large T-antigen (PKKKRKVEDP; SEQ ID NO: 349), c-Myc (PAAKRVKLD; SEQ ID NO: 350), and TUS-proteins (Kaczmarczyk S J et al. PLoS ONE 5 (1): c8889.2010). In select embodiments, the NLS comprises a c-Myc NLS.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 351), the NLS of EGL-13, MSRRRKANPTKLSENAKKLAKEVEN (SEQ ID NO: 352), the bipartite SV40 NLS, KRTADGSEFESPKKKRKV (SEQ ID NO: 353).

Any of the proteins described or referenced herein may further have an epitope tag (e.g., 3×FLAG tag, an HA tag, a Myc tag, and the like). The epitope tags may be at the N-terminus, a C-terminus, or a combination thereof of the corresponding protein. In some embodiments, the epitope tag may be adjacent, either upstream or downstream, to a nuclear localization sequence.

The effector polypeptide, NLS, or epitope tag may be appended to the proteins described herein by a linker. The linker may have any of a variety of amino acid sequences. Suitable linkers include polypeptides of between 1 amino acids and 100 amino acids in length, between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the protein. Peptide linkers with a degree of flexibility can be used. The linking peptides may have virtually any amino acid sequence, bearing in mind that the preferred linkers will have a sequence that results in a generally flexible peptide. Small amino acids, such as glycine and alanine, are generally used in creating a flexible peptide. A variety of different linkers are commercially available and are considered suitable for use, including but not limited to, glycine-serine polymers, glycine-alanine polymers, and alanine-serine polymers.

In some embodiments, the systems further comprise a guide RNA complementary to at least a portion of the target nucleic acid sequence, or a nucleic acid encoding the at least one gRNA. In addition to a sequence that binds to a target nucleic acid, in some embodiments, the gRNA may also comprise a scaffold sequence.

The gRNA or portion thereof that hybridizes to the target nucleic acid (a target site) may be any length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid.

To facilitate gRNA design, many computational tools have been developed (See Prykhozhij et al. (PLoS ONE, 10 (3): (2015)); Zhu et al. (PLOS ONE, 9 (9) (2014)); Xiao et al. (Bioinformatics. Jan. 21 (2014)); Heigwer et al. (Nat Methods, 11 (2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence. Alternatively, the gRNA and scaffold sequence may be provided as omega RNA (ωRNA). Exemplary ωRNAs are provided in the Tables herein, for example, SEQ ID NOs: 12-16, 19-20, 26-29, and 51-57. The gRNA may be a non-naturally occurring gRNA.

The system may further comprise a target nucleic acid. The terms “target sequence,” “target nucleic acid,” and “target site” (e.g., a “target genomic DNA sequence”) are used interchangeably herein to refer to a polynucleotide (nucleic acid, gene, chromosome, genome, etc.) to which a guide sequence (e.g., a synthetic guide RNA) is designed to have complementarity, wherein hybridization between the target sequence and a guide sequence promotes the formation of a complex, e.g., of the guide RNA, target, and TnpB protein, provided sufficient conditions for binding exist. The target sequence and guide sequence need not exhibit complete complementarity, provided that there is sufficient complementarity to cause hybridization and promote formation of the complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art.

The target nucleic acid may or may not be flanked by a transposon adjacent motif (TAM). A TAM can be upstream of the target sequence. In one embodiment, the target sequence is immediately flanked on the 5′end by a TAM sequence. A TAM can be 1, 2, 3, 4, 5, 6, 7, 8, 9, 10 or more nucleotides in length. In certain embodiments, a TAM is between 2-6 nucleotides in length. In some embodiments, the TAM comprises a sequence of TT(C/T)A(A/T/C). In select embodiments, the TAM sequence is TTTAT or TTCAT. In some embodiments, the TAM sequence comprises TGG. Exemplary TAM sequences are provided in the Examples herein. There may be mismatches distal from the TAM.

The target nucleic acid may or may not be flanked by a transposon-encoded motif (TEM) sequence A TEM can be downstream of the target sequence. Exemplary TEM sequences are provided in the Examples herein. In some embodiments, the target nucleic acid may be flanked by at least one end sequence.

The system may further include a donor nucleic acid. The donor nucleic acid may be a part of a bacterial plasmid, bacteriophage, a virus, autonomously replicating extra chromosomal DNA element, linear plasmid, linear DNA, linear covalently closed DNA, mitochondrial or other organellar DNA, chromosomal DNA, and the like. In some embodiments, the donor nucleic acid comprises a cargo nucleic acid sequence.

The donor nucleic acid may be flanked by at least one end sequence. In some embodiments, the donor nucleic acid is flanked on the 5′ and the 3′ end with an end sequence, e.g., at least one of a left end sequence and a right end sequence.

The term “end sequence” refers to any nucleic acid comprising a sequence capable of designating the nucleic acid between the two ends for rearrangement. Usually, these sequences contain inverted repeats and may be about 10-150 base pairs long, however the exact sequence requirements differ for the specific elements and enzymes, as demonstrated in the Examples below. End sequences may or may not include additional sequences that promotes or augment transposition.

The end sequences on either end may be the same or different. The end sequence may be the endogenous end sequences or may include deletions, substitutions, or insertions. The endogenous end sequences may be truncated. For example, for Clostridium botulinum the minimal end sequences for a variety of functions are shown in Table 6.

The donor nucleic acid, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), or greater.

The system may be a cell free system. Also disclosed is a cell comprising the system described herein. In some embodiments, the cell is a prokaryotic cell. In some embodiments, the cell is a eukaryotic cell. In some embodiments, the cell is a mammalian cell (e.g., a cell of a non-human primate or a human cell). Thus, in some embodiments, disclosed herein are systems for nucleic acid modification of a target nucleic acid sequence in a eukaryotic cell (e.g., a mammalian cell, a human cell).

### Nucleic Acids

The one or more nucleic acids encoding a TnpA protein, a TnpB protein, an IscB protein and guide RNA (e.g., ωRNA) may be any nucleic acid including DNA, RNA, or combinations thereof. In some embodiments, nucleic acids comprise one or more messenger RNAs, one or more vectors, or any combination thereof.

In some embodiments, the TnpA protein, TnpB protein and/or IscB protein and the guide RNA (e.g., ωRNA) are all encoded on the same nucleic acid. In some embodiments, each of the TnpA protein, TnpB protein, IscB protein and the guide RNA (e.g., ωRNA) are encoded on different nucleic acids. Alternatively, two or more nucleic acids encode any combination of the TnpA protein, TnpB protein and/or IscB protein and the guide RNA (e.g., ωRNA) in the system.

In certain embodiments, engineering the system for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., about 65%, about 70%, about 75%, about 80%, about 85%, about 90%, about 95%, or about 98%) of the codons encoded therein are mammalian preferred codons.

The present disclosure also provides for DNA segments encoding the proteins and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more or all of the components of the present system. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example. this may allow for DNA integration by transforming bacteria of interest, but then being left with engineered strains that have no memory of the plasmids or vectors used for the integration.

Drug selection strategies may be adopted for positively selecting for cells that underwent DNA integration. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the present system or components thereof (such as a TnpA protein, a TnpB protein, an IscB protein, and/or a guide RNA) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7 (1): 33-40; and Walther W. and Stein U., 2000 Drugs, 60 (2): 249-71, incorporated herein by reference.

In one embodiment, a DNA segment encoding a TnpA protein, a TnpB protein, an IscB protein, and/or a guide RNA (e.g., ωRNA) is contained in a plasmid vector that allows expression of the protein(s) and subsequent isolation and purification produced by the recombinant vector. Accordingly, the proteins disclosed herein can be purified following expression, obtained by chemical synthesis, or obtained by recombinant methods.

To construct cells that express the present system or components thereof, expression vectors for stable or transient expression may be constructed via conventional methods as described herein and introduced into cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, mycoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

In one embodiment, the present disclosure comprises integration of exogenous DNA into an endogenous gene. Alternatively, an exogenous DNA is not integrated into the endogenous gene. The DNA may be packaged into an extrachromosomal or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The present system (e.g., proteins, polynucleotides encoding these proteins, donor polynucleotides and compositions comprising the proteins and/or polynucleotides described herein) may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459 (1-2): 70-83), incorporated herein by reference.

### Methods

Also disclosed herein are methods for nucleic acid modification utilizing the disclosed protein, nucleic acids encoding thereof, systems, or kits.

The methods may comprise contacting a target nucleic acid sequence with a system, a protein, a nucleic acid, and/or a composition disclosed herein. The descriptions and embodiments provided above for the system, the proteins, the gRNA (e.g., ωRNA), and the nucleic acids are applicable to the methods described herein.

The phrase “modifying a nucleic acid sequence” or “nucleic acid modification” as used herein, refers to modifying at least one physical feature of a nucleic acid sequence of interest. Nucleic acid modifications include, for example, single or double strand breaks, deletion, or insertion of one or more nucleotides, and other modifications that affect the structural integrity or nucleotide sequence of the nucleic acid sequence. In some embodiments, the modifications may include cleavage of the target nucleic acid, excision of the target nucleic acid, integration of the donor nucleic acid, or a combination thereof, as described and outlined in the examples and figures provided herein.

The methods may comprise excision of a target nucleic acid sequence. For example, a system comprising TnpA may be used to site-specifically excise a target DNA sequence. In some embodiments, the TnpA is derived from a IS607-family transposon. In some embodiments, the TnpA is a serine family recombinase. In such embodiments, in addition to the TAM/TEM sequences, the target nucleic acid may further be flanked by end sequences, as described above for the donor nucleic acid.

Alternatively, the methods may comprise insertion of a donor nucleic acid. For example, systems comprising TnpA, or a combination of TnpA and TnpB, for example, may be sued for RNA-guided DNA integration.

Further, the methods may comprise cleavage of the target nucleic acid sequence. For example, a system comprising TnpB, for example, may result in RNA-guided DNA cleavage of the target nucleic acid.

IStrons may also serve as platforms for introducing selection markers, facilitating their placement within any gene, even those categorized as essential. IStrons can splice at the RNA level, resembling the characteristics of group I introns. In some embodiments, the IStrons encode TnpB or IscB and optionally TnpA or a guide RNA (e.g., ωRNA), and may further include an exogenous cargo nucleic acid (e.g., selection marker, gene of interest, etc. These elements may be used to integrate exogenous nucleic acids in a wide variety of genomic locations in a range of species (e.g., using conventional genome editing techniques) or the methods disclosed herein. Once integrated, the IS element adopts the role of an adaptive ‘gene drive’.

Thus, further provided herein are engineered group I introns comprising an exogenous nucleic acid sequence. In some embodiments, the group I intron is self-splicing. In some embodiments, the group I intron is derived from an IS607 element. In some embodiments, the group I intron is derived from Clostridium botulinum. In some embodiments, the group I intron further comprises one or more of TnpA, TnpB, IscB, or a guide RNA (e.g., ωRNA).

Modifying a nucleic acid sequence may further comprise any or all of the functions provided by the effector polypeptide as described above. For example, any of the TnpA, TnpB, or IscB may be provided with a linked or conjugated effector polypeptide which will modify the target nucleic acid sequence accordingly. In some embodiments, the TnpA, TnpB, or IscB are provided as a fusion protein. Alternatively, TnpA, TnpB, or IscB include a binding moiety which associates with a moiety on the effector polypeptide to form a conjugate in situ.

The target nucleic acid sequence may be in a cell. In some embodiments, the contacting a target nucleic acid sequence comprises introducing the system, composition, or proteins into the cell. As described above the system, composition, or proteins may be introduced into eukaryotic or prokaryotic cells by methods known in the art. In some embodiments, the cell is a mammalian cell. In some embodiments, the cell is a human cell.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

Polynucleotides containing the target nucleic acid sequence may include, but is not limited to, purified chromosomal DNA, total cDNA, cDNA fractionated according to tissue or expression state (e.g., after heat shock or after cytokine treatment other treatment) or expression time (after any such treatment) or developmental stage, plasmid, cosmid, BAC, YAC, phage library, etc. Polynucleotides containing the target site may include DNA from organisms such as Homo sapiens, Mus domesticus, Mus spretus, Canis domesticus, Bos, Caenorhabditis elegans, Plasmodium falciparum, Plasmodium vivax, Onchocerca volvulus, Brugia malayi, Dirofilaria immitis, Leishmania, Zea maize, Arabidopsis thaliana, Glycine max, Drosophila melanogaster, Saccharomyces cerevisiae, Schizosaccharomyces pombe, Neurospora, Escherichia coli, Salmonella typhimurium, Bacillus subtilis, Neisseria gonorrhoeae, Staphylococcus aureus, Streptococcus pneumonia, Mycobacterium tuberculosis, Aquifex, Thermus aquaticus, Pyrococcus furiosus, Thermus littoralis, Methanobacterium thermoautotrophicum, Sulfolobus caldoaceticus, and others.

The methods may comprise administering to the subject, in vivo, or by transplantation of ex vivo treated cells, an effective amount of the described system. In some embodiments, the vector(s) is delivered to the tissue of interest by, for example, an intramuscular, intravenous, transdermal, intranasal, oral, mucosal, or other delivery methods.

The proteins, composition, components of the present system or ex vivo treated cells may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

In some embodiments, an effective amount of the components of the present system or compositions as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful DNA modification is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. Sec, e.g., Remington: The Science and Practice of Pharmacy 20th Ed. (2000) Lippincott Williams and Wilkins, Ed. K. E. Hoover.

The methods may be used for a variety of purposes. For example, the methods may include, but are not limited to, inactivation of a microbial gene, RNA-guided DNA integration in a plant or animal cell, methods of treating a subject suffering from a disease or disorder. The disclosed methods may modify a target DNA sequence in a cell so as to modulate expression of the target DNA sequence, e.g., expression of the target DNA sequence is increased, decreased, or completely eliminated (e.g., via deletion of a gene). The modifications of the target sequence may lead to, for example, gene correction, gene replacement, gene tagging, transgene insertion, nucleotide deletion/addition/correction, gene disruption, gene mutation, gene knock-down, etc.

In some embodiments, the methods described herein may be used to genetically modify a plant or plant cell. As used herein, genetically modified plants include a plant into which has been introduced an exogenous polynucleotide. Genetically modified plants also include a plant that has been genetically manipulated such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetically modified plant is one having an altered regulatory sequence, such as a promoter, to result in increased or decreased expression of an operably linked endogenous coding region. The genetically modified plant may promote a desired phenotypic or genotypic plant trait.

Genetically modified plants can potentially have improved crop yields, enhanced nutritional value, and increased shelf life. They can also be resistant to unfavorable environmental conditions, insects, and pesticides. The present systems and methods have broad applications in gene discovery and validation, mutational and cisgenic breeding, and hybrid breeding. The present methods may facilitate the production of a new generation of genetically modified crops with various improved agronomic traits such as herbicide resistance, herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, disease (e.g. bacterial, fungal, and viral) resistance, high yield, and superior quality. The present methods may also facilitate the production of a new generation of genetically modified crops with optimized fragrance, nutritional value, shelf-life, pigmentations (e.g., lycopene content), starch content (e.g., low-gluten wheat), toxin levels, propagation and/or breeding and growth time. See, for example, CRISPR/Cas Genome Editing and Precision Plant Breeding in Agriculture (Chen et al., Annu Rev Plant Biol. 2019 Apr. 29; 70:667-69), incorporated herein by reference.

The present method may confer one or more of the following traits to the plant cell: herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, resistance to fungal disease, and resistance to viral disease.

The present disclosure provides for a modified plant cell produced by the present method, a plant comprising the plant cell, and a seed, fruit, plant part, or propagation material of the plant. Transformed or genetically modified plant cells of the present disclosure may be as populations of cells, or as a tissue, seed, whole plant, stem, fruit, leaf, root, flower, stem, tuber, grain, animal feed, a field of plants, and the like. The present disclosure provides a transgenic plant. The transgenic plant may be homozygous or heterozygous for the genetic modification. Also provided by the present disclosure are transformed or genetically modified plant cells, tissues, plants, and products that contain the transformed or genetically modified plant cells. The present disclosure further encompasses the progeny, clones, cell lines or cells of the transgenic plants.

The present system and method may be used to modify a plant stem cell. The present disclosure further provides progeny of a genetically modified cell, where the progeny can comprise the same genetic modification as the genetically modified cell from which it was derived. The present disclosure further provides a composition comprising a genetically modified cell.

In one embodiment, the transformed or genetically modified cells, and tissues and products comprise a nucleic acid integrated into the genome, and production by plant cells of a gene product due to the transformation or genetic modification.

Methods of introducing exogenous nucleic acids into plant cells are well known in the art. Such plant cells are considered “transformed.” DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation. The transformation can be transient or stable transformation. Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (i.e., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. Sec., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used to produce a transgenic plant. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

In one embodiment, the present methods may be adapted to use in plants. The vectors may be optimized for transient expression of the present system in plant protoplasts, or for stable integration and expression in intact plants via the Agrobacterium-mediated transformation.

In certain embodiments, the present methods use a monocot promoter to drive the expression of one or more components of the present systems (e.g., gRNA) in a monocot plant. In certain embodiments, the present methods use a dicot promoter to drive the expression of one or more components of the present systems (e.g., gRNA) in a dicot plant.

The present methods may be used with various microbial species, including human pathogens that are medically important, and bacterial pests that are key targets within the agricultural industry, as well as antibiotic resistant versions thereof. The method may be designed to target any gene or any set of genes, such as virulence or metabolic genes, for clinical and industrial applications in other embodiments. The present systems and methods may be used to inactivate microbial genes. In some embodiments, the gene is an antibiotic resistance gene.

The methods described here also provide for treating a disease or condition in a subject. The methods may comprise administering to the subject, in vivo, or by transplantation of ex vivo treated cells (e.g., disclosed T cells), a therapeutically effective amount of the present system, polypeptides, or components thereof.

In some embodiments, the methods are used to treat a pathogen or parasite on or in a subject by altering the pathogen or parasite. In some embodiments, the methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutancous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1 (1): 192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the target DNA sequence can comprise a cancer oncogene. The present disclosure provides for gene editing methods that can ablate a disease-associated gene (e.g., a cancer oncogene), which in turn can be used for in vivo gene therapy for patients. In some embodiments, the gene editing methods include donor nucleic acids comprising therapeutic genes.

### Kits

Also within the scope of the present disclosure are kits that include the components of the present system, such as a TnpA protein, a TnpB protein, an IscB protein, and/or a guide RNA (e.g., ωRNA).

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system. The device may include an infusion device, an intravenous solution bag, a hypodermic needle, a vial, and/or a syringe.

### EXAMPLES

The following are examples of the present invention and are not to be construed as limiting.

### Materials and Methods

IscB and TnpB detection and database curation. Homologs of IscB proteins were comprehensively detected using the amino acid sequence of a K. racemifer homolog (NCBI Accession: WP_007919374.1) as the seed query in a JackHMMER part of the HMMER suite (v3.3.2). To minimize false homologs, a conservative inclusion and reporting threshold of 1e-30 was used in the iterative search against the NCBI NR database (retrieved on Jun. 11, 2021), resulting in 5,715 hits after convergence. These putative homologs were then annotated to profiles of known protein domains from the Pfam database (retrieved on Jun. 29, 2021) using hmmscan with an E-value threshold of 1e-5. Proteins that did not contain the RRXRR, RuvC, RuvC_III, or the RuvX domain were discarded. Although the HNH domain was annotated, proteins without the HNH were not removed. The variation in the presence of the HNH domain was preserved to better represent the natural diversity of IscBs. From the remaining set, proteins that were less 250 aa were removed to eliminate partial or fragmented sequences, resulting in a database of 4,674 non-redundant IscB homologs. Contigs of all putative iscB loci were retrieved from NCBI for downstream analysis using the Bio.Entrez package.

TnpB homologs were comprehensively detected similarly to IscB, use both the H. pylori (HpyTnpB) amino acid sequence (NCBI Accession: WP_078217163.1) and the G. stearothermophilus (Gs/TnpB2) amino acid sequence (NCBI Accession: WP_047817673.1) as seed queries for two independent iterative jackhammer searches against the NR database, with an inclusion and reporting threshold of 1e-30. The union of the two searches were taken, and proteins that were less than 250 aa were removed to trim partial or fragmented sequences, resulting in a database of 95,731 non-redundant TnpB homologs. Contigs of all putative tnpB loci were retrieved from NCBI for downstream analysis using the Bio.Entrez package.

Protein sequences used for the G. stearothermophilus proteins in the examples below are shown in Table 1.

Phylogenetic analyses. IscB protein sequences were clustered with at least 95% length coverage and 95% alignment coverage using CD-HIT (v4.8.1). The clustered representatives were taken and aligned using MAFFT (v7.508) with the E-INS-I method for 4 rounds. Post-alignment cleaning consisted of using trimA1 (v1.4.rev15) to remove columns containing more than 90% of gaps and manual inspection. The phylogenetic tree was created using IQ-Tree 2 (v2.1.4) with the WAG model of substitution. Branch support was evaluated with 1000 replicates of SH-aLRT, aBayes, and ultrafast bootstrap support from the IQTREE package. The tree with the highest maximum-likelihood was used as the reconstruction of the IscB phylogeny.

Putative TnpB sequences were clustered by 50% length coverage and 50% alignment coverage using CD-HIT. Similar to IscB, the clustered representatives were taken and aligned using MAFFT⁵⁵ with the E-INS-I method for 4 rounds. Post-alignment cleaning consisted of using trimA1 to remove columns containing more than 90% of gaps and manual inspection. The phylogenetic tree was created using IQ-Tree 2 with the WAG model of substitution. Branch support was evaluated with 1000 replicates of SH-aLRT, aBayes, and ultrafast bootstrap support from the IQTREE package. The tree with the highest maximum-likelihood was used as the reconstruction of the TnpB phylogeny.

ωRNA covariation analyses. Initially searches of the Rfam database indicated a potential ncRNA belonging to the HNH endonuclease-associated RNA and ORF (HEARO) RNA (RF02033). A covariance model of HEARO RNA (retrieved Jun. 24, 2021) was initially used to discover all HEAROs within the curated IscB-associated contig database using cmsearch from the Infernal package (v1.1.4). A liberal minimum bit score of 15 was used in an attempt to capture distant or degraded HEAROs, and the identification of a HEARO as a putative ωRNA was supported by its proximity, orientation, and relative location to the nearest identified IscB ORF. Remaining hits were considered ωRNAs if they were upstream of an IscB ORF and within 500 bp or overlapping with the nearest IscB ORF. After inspecting the RF02033 model, it appeared to lack additional structural elements located downstream. To address this, the boundaries of ωRNA were refined and used to generate a more accurate, comprehensive covariance model. Hits to the RF02033 model described above were retrieved, expanded 200 bp downstream, and clustered by 80% length coverage and 80% alignment coverage using CD-HIT. CMfinder (v0.4.1.9) was then used with recommended parameters to discover new motifs de novo. Additional structures were discovered and present in over 80% of the expanded sequences. This covariance model was used to expand the 3′ coordinates of previously identified ωRNAs to encompass the second stem loop using cmsearch on expanded ωRNAs. These refined ωRNA boundaries and sequences were then used to create a new ωRNA model. The refined ωRNAs were clustered by 99% length coverage and 99% alignment coverage using CD-HIT to remove duplicates. A structure-based multiple alignment was then performed using mLocARNA (v1.9.1) with the following parameters:
--max-diff-am 25--max-diff 60--min-prob 0.01--indel-50--indel-open-750--plfold-span 100--alifold-consensus-dp

The resulting alignment with structural information was used to generate a new ωRNA covariance model with the Infernal suite, refined with Expectation-Maximization from CMfinder, and verified with R-scape at an E-value threshold of 1e-5. The resulting ωRNA covariance model was used with cmsearch to discover new ωRNAs within the curated IscB-associated contig database. The resulting sequences were aligned to generate a new CM model that was used to again search the IscB-associated contig database. This process was repeated three times for the final generic IscB-associated ωRNA model.

While covariance models of TnpB-associated ωRNAs were available through Rfam (RF03065) and (RF02998), these models appeared to only include a very small subset of TnpB-associated ωRNA and contained very few hits. Based on small RNA-seq analysis that suggested a ncRNA often overlapped with the TnpB ORF and extending into the RE boundary of the IS element, sequences 150 bp downstream of the last nucleotide of the TnpB ORF were extracted to define the RE and transposon boundaries. The ˜ 150-bp sequences were clustered by 99% length coverage and 99% alignment coverage using CD-HIT to remove duplicates. The remaining sequences were then clustered again by 95% length coverage and 95% alignment coverage using CD-HIT. This was done to identify clusters of sequences that were closely related but not identical, as expected of IS elements that have recently mobilized to new locations. For the 300 largest clusters, which all had a minimum of 10 sequences, MUSCLE (v3.8.1551) with default parameters was used to align each cluster of sequences. Then, each cluster alignment was manually inspected for the boundary between high conservation and low conservation, or where there was a stark drop-off in mean pairwise identity over all sequences. This point was annotated for each cluster as the putative 3′ end of the IS elements. If there was no conservation boundary, sequences in these clusters were expanded by another 150 bp, in order to capture the transposon boundaries, and realigned. The consensus sequence of each alignment (defined by a 50% identity threshold up until the putative 3′ end) was extracted, and rare insertions that introduced gaps in the consensus were manually removed. With the 3′ boundary of the IS element, and thus the 3′ boundary of the TnpB ωRNA properly defined, a covariance model of the TnpB ωRNA could be built.

From a randomly selected member of each of the 300 clusters, a 250-bp window of sequence 5′ of the 3′ end of the ωRNA was extracted. A structurally based multiple alignment was then performed using mLocARNA and used to generate a TnpB-specific ωRNA covariance model with Infernal, refined with CMfinder, and verified with R-scape at an E-value threshold of 1e-5. This was iterated twice to generate the final generic model of TnpB-associated ωRNA. In addition, more localized ωRNA covariance models were created for each of the 4 TnpB homologs used in this study (GstTnpB1-4). Each protein was used as a seed query in a phmmer (v3.3.2) search against the NR database, with an inclusion and reporting threshold of 1e-30 to identify close relatives of each protein. The steps described above were used to define transposon boundaries and generate ωRNA models using sequences identified in the phmmer search.

TnpA detection and autonomous element identification. For both IscB- and TnpB-associated contigs, TnpA was detected using the Pfam Y1_Tnp (PF01797) for a hmmsearch from the HMMR suite (v3.3.2), with an E-value threshold of 1e-4. This search was performed independently on both the curated CDSs of each contig from NCBI and the ORFs predicted by Prodigal on default settings. The union of these searches was used as the final set of detected TnpA proteins. IS elements that encoded IscB homologs within 1,000 bp of a detected TnpA, or that encoded TnpB homologs within 10,000 bp of a detected TnpA, were defined as autonomous. Analysis which uncovered association with serine resolvases (PF00239) was performed with the same parameters mentioned above.

Orientation bias analysis. The closest NCBI-annotated/predicted CDS upstream of each transposon-encoded gene (tnpBliscB or the IS630 transposase) was retrieved and analyzed relative to the gene itself. Initially, the metadata for every NCBI-annotated CDS within contigs containing these genes (tnpBliscB or IS630) were retrieved, including coordinates and strandedness. Using this information, the closest upstream CDS was identified for each gene based on distance. Then, the annotated orientation of the closest upstream CDS was compared to the annotated orientation of the respective transposon-encoded gene (tnpBliscB or IS630), to determine whether they were matching. This analysis was performed for gene/CDS pairs at all distances between 0-1000 bp upstream (5′) of the transposon-encoded gene ORF, where 0-bp was defined as overlapping, using a custom Python script.

Transposon boundary and TAM/TEM motif determination for G. stearothermophilus IS elements (ISGst). IS200/IS605 elements found in G. stearothermophilus strain DSM 458 (NCBI Accession: NZ_CP016552.1) that encoded iscB or tnpB were identified by a protein homology-based search, as described above. Initial identification of transposon boundaries ware identified by multiple sequence alignment of each unique tnpB or iscB gene using DNA sequences flanking the TnpB/IscB ORF, and were aligned using MUSCLE (5.1) PPP algorithm in Geneious (2023.0.1). To build covariance models of the transposon ends, cmfinder was used to detect structural motifs for each end of ISGst1, ISGst2 and ISGst5 (LE and RE separately) and produce an alignment based on secondary structure. This model was then used for further searches (CMSearch), to identify structurally similar positions within the genome of G. stearothermophilus strain DSM 458. All transposon ends were initially paired with the most similar query end and then manually curated, to ensure each the LE and RE within a given pair were correctly positioned relative to each other. This analysis identified several PATE-like elements lacking any protein-coding genes, and a total of 47 IS elements were identified with similar LE and RE sequences. 50 bp upstream and downstream were extracted and aligned using MUSCLE (5.1) PPP algorithm in Geneious and trimmed using trimA1 (v1.4.rev15), to capture transposon boundaries and identify TAM and TEM motifs based on previous literature describing the location of these essential motifs. Transposon DNA guide regions were predicted based on structural similarities to the transposon ends of H. pylori IS608 and covarying mutations at those predicted locations. TAM motifs, which function as target sites for the transposon insertion event, were confirmed by blastn analysis of DNA sequences flanking predicted transposon boundaries to the NT or WGS database. Phylogenetic trees of transposon ends were built using FastTrec (2.1.11) with default parameters.

Small RNA-seq analyses. Small RNA-seq reads were retrieved from NCBI SRA database under accession SRX3260293. Reads were downloaded using the SRA toolkit (2.11.0) and mapped to genomic regions encoding G. stearothermophilus IscB and TnpB homologs used in this study, using G. stearothermophilus strain ATCC 7953 (GCA_000705495.1) from which small RNA-seq data derives. Reads were mapped using Geneious RNA assembler at medium sensitivity and visualized using Integrative Genomics Viewer.

Plasmid construction. All plasmids used in this study are described in Tables 7 and 8. In brief, genes encoding TnpA, TnpB, and IscB homologs from G. stearothermophilus, H. pylori and D. radiodurans were synthesized by GenScript, along mini-Tn elements containing a chloramphenicol resistance gene. To generate mini-Tn plasmids, gene fragments (GenScript) encoding the transposase (TnpA) downstream of a lac and T7 promoter, and transposon ends flanking a chloramphenicol resistance gene, were cloned into EcoRI sites of pUC57. To generate pEffector plasmids, gene fragments (Genscript) of ωRNA encoded downstream of T7 promoter, along with tnpB or iscB also encoded downstream of T7 promoter, were cloned into pCDF-Duet1 vectors at PfoI and Bsu36I sites. Oligonucleotides containing J23-series promoters were cloned into SalI and KpnI sites, replacing the T7 promoter for ωRNA expression, or into PfoI-XhoI sites, replacing the T7 promoter for tnpB expression. pTarget plasmids were generated using a minimal pCOLADuet-1, generated by around-the-horn PCR to create a minimal pCOLA-Duet-1 containing only the ColA origin of replication and kanamycin resistance gene. This vector was then used to generate pTargets encoding 45-bp target sites by around-the-horn PCR. Derivatives of these plasmids were cloned using a combination of methods, including Gibson assembly, restriction digestion-ligation, ligation of hybridized oligonucleotides, and around-the-horn PCR. Plasmids were cloned, propagated in NEB Turbo cells (NEB), purified using Miniprep Kits (Qiagen), and verified by Sanger sequencing (GENEWIZ).

Recombineering. Lambda Red (2-Red) recombination was used to generate genomically integrated mini-Tn cassette. In brief, E. coli strain MG1655 (sSL0810) was transformed with pSIM6 (pSL2684) carrying a temperature-sensitive vector encoding λ-Red recombination genes, generating strain sSL2681, and cells were made electrocompetent using standard methods. Fragments for recombineering were generated using standard PCR amplification with primers to generate 50-bp overhangs homologous to the sites of integration. PCR fragments were gel extracted and used to electroporate sSL2681, and cells were recovered for 24 h in LB media. Cells were spun down and plated onto LB-agar containing kanamycin (50 μg ml⁻¹) to select for mini-Tn cassette integration. Single colonies were isolated and confirmed to contain a genomically integrated mini-Tn within the lacZ locus by colony PCR and Sanger sequencing.

Transposon excision assays. For each excision experiment involving a plasmid-based IS element, a single plasmid encoding for TnpA and a chloramphenicol resistance gene-containing mini-Tn IS element was used to transform E. coli strain MG1655. Cultures were grown overnight at 37° C. on LB-agar under antibiotic selection (100 μg ml⁻¹ carbenicillin, 25 μg ml⁻¹ chloramphenicol). Next, three colonies were picked from each agar plate and used to inoculate 5 ml LB supplemented with 0.05 mM IPTG and antibiotic for only for backbone marker (100 μg ml⁻¹ carbenicillin). The liquid cultures were incubated at 37° C. for 24 h. Cell lysates were generated, as described previously (Klompe, S. E., et al., Nature 571, 219-225, doi: 10.1038/s41586-019-1323-z (2019)). In brief, the optical density at 600 nm was measured for liquid cultures. Approximately 3.2×10⁸ cells (equivalent to 200 μl of OD₆₀₀=2.0) were transferred to a 96-well plate. Cells were pelleted by centrifugation at 4,000 g for 5 min and resuspended in 80 μl of H₂O. Next, cells were lysed by incubating at 95° C. for 10 min in a thermal cycler. The cell debris was pelleted by centrifugation at 4,000 g for 5 min, and 10 μl of lysate supernatant was removed and serially diluted with 90 μl of H₂O to generate 10- and 100-fold lysate dilutions for PCR and qPCR analysis.

IS element excision from the plasmid backbone was detected by PCR using OneTaq 2× Master Mix with Standard Buffer (NEB) and 0.2 μM primers, designed to anneal upstream and downstream of the IS element. PCR reactions contained 0.5 μl of each primer at 10 μM, 12.5 μl of OneTaq 2× MasterMix with Standard Buffer, 2 μl of 100-fold diluted cell lysate serving as template, and 9.5 μl of H₂O. The total volume per PCR was 25 μl. Measurements were performed in a BioRad T100 thermal cycler using the following thermal cycling parameters: DNA denaturation (94° C. for 30 s), 35 cycles of amplification (annealing: 52° C. for 20 s, extension: 68° C. for 30 s), followed by a final extension (68° C. for 5 min). Products were resolved by 1.5% agarose gel electrophoresis and visualized by staining with SYBR Safe (Thermo Fisher Scientific). IS element excision events were confirmed by Sanger sequencing of gel-extracted, column-purified (Qiagen) PCR amplicons (GENEWIZ/Azenta Life Sciences).

For excision events involving genomically integrated IS elements, lysate was prepared as described above but harvested from LB-agar containing carbenicillin (100 μg ml⁻¹), spectinomycin (100 μg ml⁻¹), and X-gal (200 mg ml 1) in transposition assays combining TnpA and TnpB, as described below. Measurements were performed in a BioRad T100 thermal cycler using the following thermal cycling parameters: DNA denaturation (94° C. for 30 s), 26 cycles of amplification (annealing: 52° C. for 20 s, extension: 68° C. for 1:15 min), followed by a final extension (68° C. for 5 min). Products were resolved by 1.5% agarose gel electrophoresis and visualized by staining with SYBR Safe (Thermo Fisher Scientific). IS element excision events were confirmed by Sanger sequencing of gel-extracted, column-purified (Qiagen) PCR amplicons (GENEWIZ/Azenta Life Sciences).

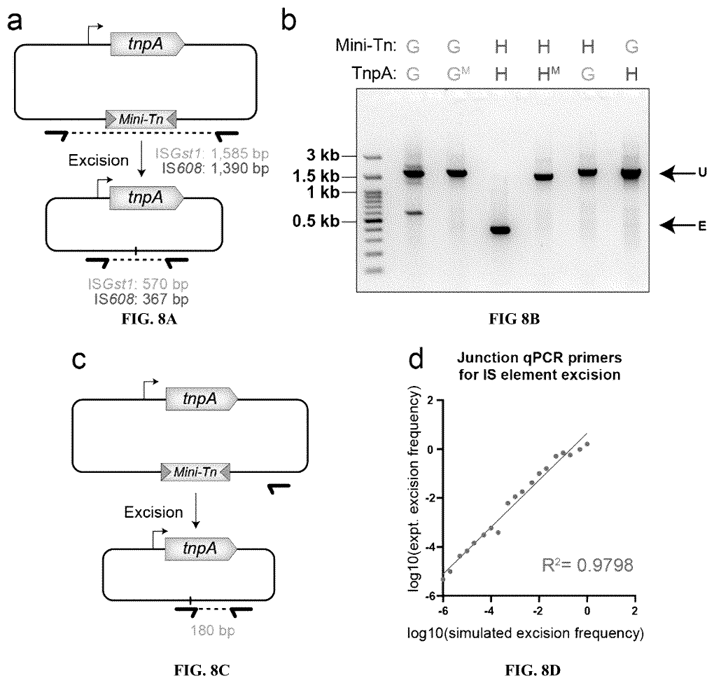

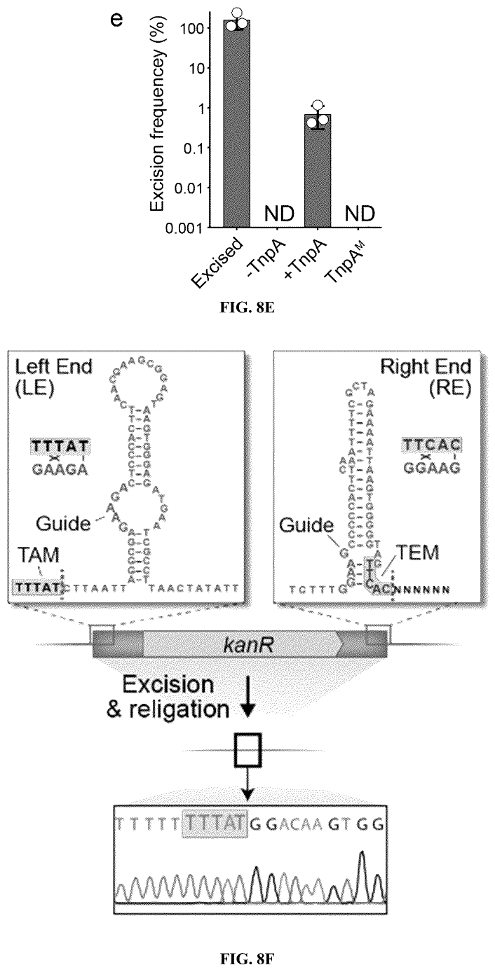

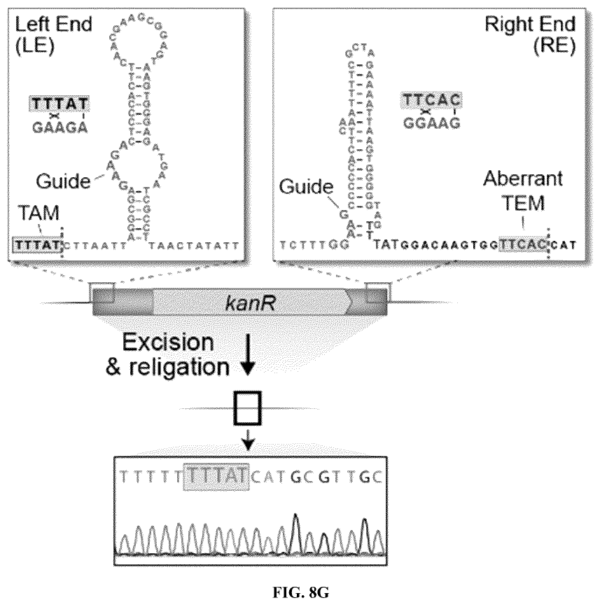

qPCR quantification of IS element excision. IS element excision frequency from a plasmid backbone was detected by qPCR using SsoAdvanced™ Universal SYBR Green Supermix. qPCR analysis (FIGS. 8C-8E) was performed using a donor joint-specific primer along with a flanking primer designed to amplify only the excision product; genome-specific primers for relative quantification were designed to amplify the E. coli reference gene, rssA. 10 μl qPCR reactions containing 5 μl of SsoAdvanced™ Universal SYBR Green Supermix, 2 μl of 2.5 μM primer pair, 1 μl H₂O, and 2 μl of tenfold-diluted lysate were prepared as described for transposon excision assays. Reactions were prepared in 384-well clear/white PCR plates (BioRad), and measurements were performed on a CFX384 RealTime PCR Detection System (BioRad) using the following thermal cycling parameters to selectively amplify excision products: polymerase activation and DNA denaturation (98° C. for 2.5 min), 40 cycles of amplification (98° C. for 10 s, 62° C. for 20 s), and terminal melt-curve analysis (65-95° C. in 0.5° C. per 5 s increments).

To confirm the sensitivity of qPCR-based measurements from plasmid encoded mini-Tn substrates, lysates were prepared from cells harboring a plasmid containing a mock excised mini-Tn substrate (pSL4826) and a plasmid containing the mini-Tn but lacking an active TnpA transposase required for excision (pSL4735). Variable IS element excision frequencies were simulated across five orders of magnitude (ranging from 0.002% to 100%) by mixing cell lysates the control strain and the IS-encoding strain in various ratios, which demonstrated accurate detection of excision products in genomic IS element excision assays in vivo to a frequency of 0.001 (FIG. 8D).

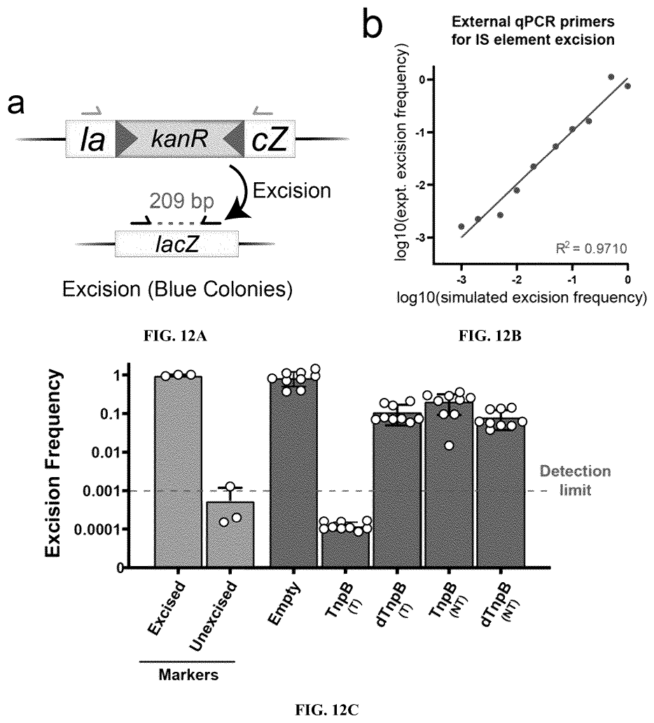

Similarly, IS element excision frequencies of genomically integrated mini-TN were quantified by qPCR using SsoAdvanced™ Universal SYBR Green Supermix (BioRad) (FIG. 12). Cells were harvested from LB containing carbenicillin (100 μg ml⁻¹), spectinomycin (100 μg ml⁻¹), and X-gal (200 mg ml⁻¹), as described above. qPCR analysis was performed using transposon flanking- and genome-specific primers. Transposon flanking primers were designed to amplify an approximately 209-bp fragment upon excision. An unexcised product would yield 1,661 bp unexcised fragment. A separate pair of genome-specific primers was designed to amplify an E. coli reference gene (rssA) for normalization purposes. 10 μl qPCR reactions containing 5 μl of SsoAdvanced™ Universal SYBR Green Supermix, 2 μl of 2.5 μM primer pair, 1 μl H₂O, and 2 μl of tenfold-diluted lysate were prepared, as described for transposon excision assays. Reactions were prepared in 384-well clear/white PCR plates (BioRad), and measurements were performed on a CFX384 RealTime PCR Detection System (BioRad) using the following thermal cycling parameters to selectively amplify excision products: polymerase activation and DNA denaturation (98° C. for 2.5 min), 40 cycles of amplification (98° C. for 10 s, 60° C. for 20 s), and terminal melt-curve analysis (65-95° C. in 0.5° C. per 5 s increments).

To confirm the sensitivity of qPCR-based measurements from genomically integrated mini-Tn, lysates were prepared from a control MG1655 strain, and a strain containing a genomically-encoded IS element that disrupts the lacZ gene. Similar to the plasmid-based assay, variable IS element excision frequencies were simulated across five orders of magnitude (ranging from 0.002% to 100%) by mixing cell lysates the control strain and the IS-encoding strain in various ratios, and showed accurate detection of excision products in genomic IS element excision assays in vivo to a frequency of 0.001 (FIG. 12B).

Mating-out assays. sSL1592, harbors a mini-F plasmid derivative with an integrated spectinomycin cassette. This strain was transformed with a plasmid carrying a mini-Tn harboring a kanamycin marker and either GstTnpA (pSL4245) or catalytically inactive GstTnpA (pSL4974). Cells were selected on LB media containing spectinomycin (100 μg ml⁻¹), carbenicillin (100 μg ml⁻¹), and kanamycin (50 μg ml⁻¹) to generate a donor strain. Three independent colonies were inoculated in liquid LB media containing spectinomycin (100 μg ml⁻¹), carbenicillin (100 μg ml⁻¹), kanamycin (50 μg ml⁻¹), and 0.05 mM IPTG to induce expression of TnpA for 12 h at 37° C. In parallel, the recipient strain harboring genomically encoded resistance for rifampicin and nalidixic acid were grown in liquid LB media containing rifampicin (100 μg/mL) and nalidixic acid (30 μg/mL) for 12 h at 37° C. Cells were 100-fold diluted into fresh liquid LB media with respective antibiotics and grown for 2 h to ˜0.5 OD. Cells were then washed with H₂O and mixed at a concentration of 5×10⁷ for both donor and recipient cells, and plated onto solid LB-agar media with no antibiotic selection. Cells were grown for 20 h at 37° C., scraped off plates, and resuspended in H₂O. Cells were then serially diluted and plated onto LB media containing rifampicin (100 μg/mL), nalidixic acid (30 μg/mL), spectinomycin (100 μg ml⁻¹), and kanamycin (50 μg ml⁻¹) to monitor transposition. In addition, cells were also plated to rifampicin (100 μg ml⁻¹), nalidixic acid (30 μg ml⁻¹), and spectinomycin (100 μg ml⁻¹), to determine the entire transconjugant population. The frequency of transposition was calculated by taking the number of colonies that exhibited Nal^{R}+Rif^{R}+Spec^{R}+Kan^{R} phenotype (e.g., transposition positive), divided by the number of transconjugants that exhibited a Nal^{R}+Rif^{R}+SpecR phenotype. Transconjugants showing resistance to nalidixic acid, rifampicin, spectinomycin, and kanamycin were isolated using Zymo Research ZR BAC DNA miniprep kit and sequenced using nanopore long-read sequencing (Plasmidsaurus). Reads were analyzed in Geneious Prime (2023.0.1) by using a custom blast database to identify reads containing mini-Tn and flanking mini-F plasmid sequence. Insertion events were aligned to Mini-F plasmid reference to identify sites of integration.

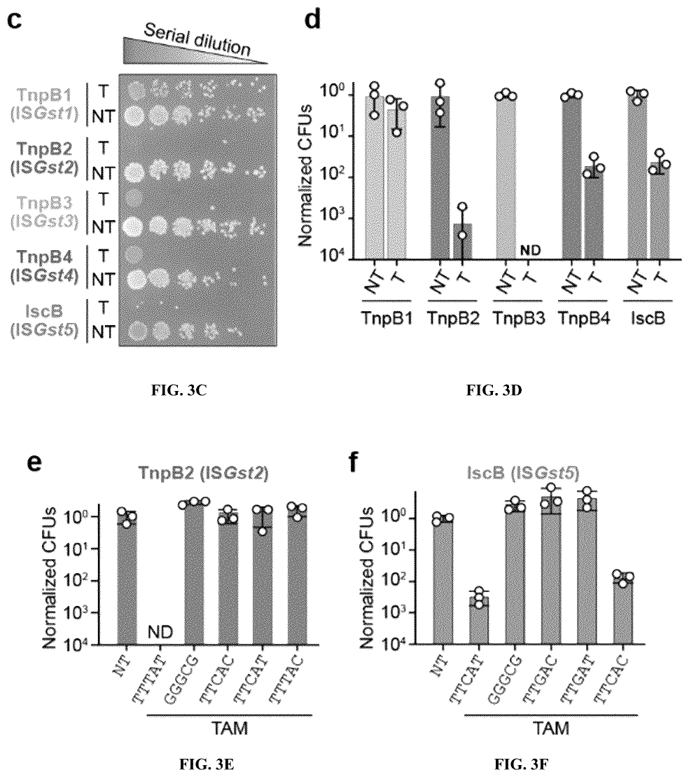

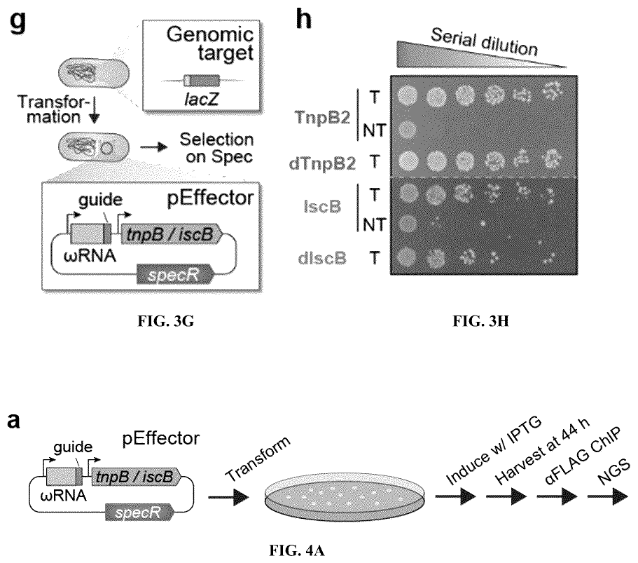

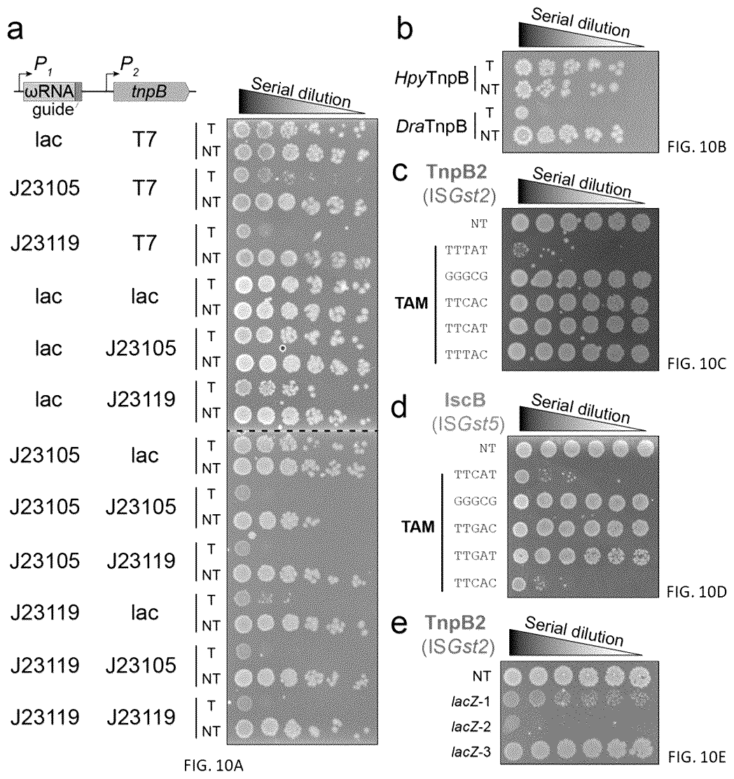

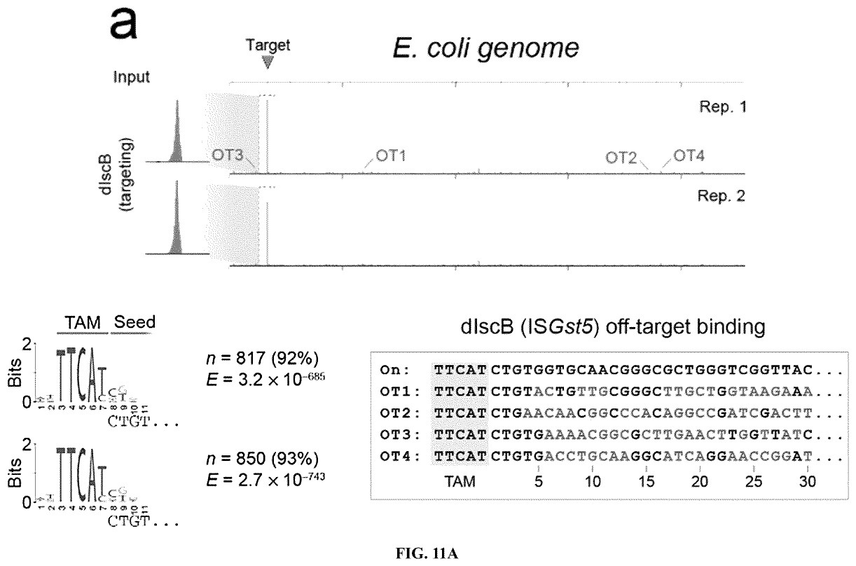

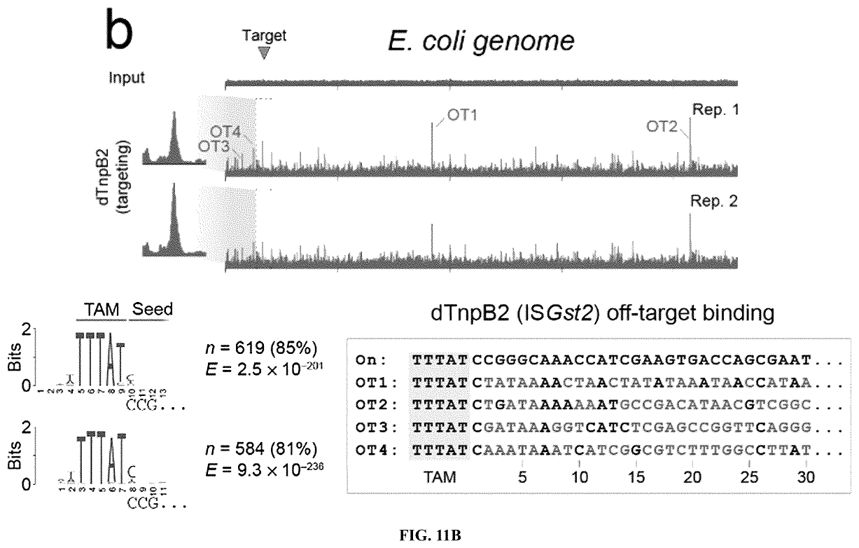

Plasmid interference assays. Plasmid interference assays were performed in E. coli BL21 (DE3) (FIGS. 3C, 3F, 10A-10B, and 10D) or E. coli str. K-12 substr. MG1655 (sSL0810) strains for all other experiments. For FIG. 3C (TnpB homologs), BL21 (DE3) cells were transformed with pTarget plasmids, and single colony isolates were selected to prepare chemically competent cells. 400 ng of pEffector plasmids were then delivered via transformation. After 3 h, cells were spun down at 4000 g for 5 min and resuspended in 20 μl of H₂O. Cells were then serial diluted (10×) and transferred to LB media containing spectinomycin (100 μg ml⁻¹), kanamycin (50 μg ml⁻¹), and 0.05 mM IPTG and grown for 24 h at 37° C. For all remaining spot assays using MG1655 strains, chemically competent cells were first prepared with pEffector plasmid and then transformed with 400 ng of pTarget plasmids. After 3 h, cells were spun down at 4000 g for 5 min and resuspended in 20 μl of H₂O. Cells were then serial diluted (10×) and transferred to LB media containing spectinomycin (100 μg ml⁻¹), kanamycin (50 μg ml⁻¹), and 0.05 mM IPTG and grown for 14 h at 37° C. Plates were imaged in an Amersham Imager 600.

Quantification of plasmid interference was calculated by determining the number of colony forming units (CFUs) following transformation. Cells were first transformed with pEffector plasmids and prepped as chemically competent cells for a second round of transformation with 200 ng of pTarget. Cells were then spun down at 4000 g for 5 min and resuspended in 100 μL of H₂O. Cells were then serial diluted and plated to LB media containing spectinomycin (100 μg ml⁻¹), kanamycin (50 μg ml⁻¹). 0.05 mM IPTG was added to media when T7 promoter was used. CFUs were counted following 24 h of growth at 37° C. Frequencies were normalized relative to a non-targeting guide RNA.

Genome targeting and cell killing assays. Cell killing assays via genomic targeting with TnpB (FIGS. 3H and 10E) or IscB (FIG. 3H) were performed by transforming E. coli str. K-12 substr. MG1655 (sSL0810) strains with spectinomycin-resistant plasmids constitutively expressing TnpB/IscB and either genomic targeting or non-targeting guide RNAs. Cells were transformed with 400 ng plasmid. After 3 h, cells were spun down at 4000 g for 5 min and resuspended in 20 μl of H₂O. Cells were then serial diluted (10×) and transferred to LB media containing spectinomycin (100 μg ml⁻¹) and grown for 24 h at 37° C.

ChIP-seq experiments and library preparation. ChIP-seq experiments were generally performed as described previously (Sec, Hoffmann, F. T. et al., Nature 609, 384-393 (2022), incorporated herein by reference). The following active site mutations were introduced to inactivate the endonuclease domains of the respective 3×Flag-tagged proteins to simulate DNA binding prior to DNA cleavage: GstIscB (D87A, H₂₃₈A, H₂₃₉A); GstTnpB (D196A); SpyCas9 (D10A, H₈₄₀A); AsCas12a (D908A). E. coli BL21 (DE3) cells were transformed with a single plasmid encoding the catalytically inactive effector and either a lacZ targeting ωRNA or non-targeting ωRNA. After incubation for 16 h at 37° C. on LB agar plates with antibiotics (200 μg ml⁻¹ spectinomycin), cells were scraped and resuspended in 1 ml of LB. The optical density at 600 nm (OD600) was measured, and approximately 4.0×10⁸ cells (equivalent to 1 ml with an OD600 of 0.25) were spread onto two LB agar plates containing antibiotics (200 μg ml⁻¹ spectinomycin) and supplemented with 0.05 mM IPTG. Plates were incubated at 37° C. for 24 h. All cell material from both plates was scraped and transferred to a 50 ml conical tube.

Cross-linking was performed by mixing 1 ml of formaldehyde (37% solution; Thermo Fisher Scientific) to 40 ml of LB medium (˜1% final concentration) followed by immediate resuspension of the scraped cells by vortexing and 20 min of gentle shaking at room temperature. Cross-linking was stopped by the addition of 4.6 ml of 2.5 M glycine (˜0.25 M final concentration) followed by 10 min incubation with gentle shaking. Cells were pelleted at 4° C. by centrifuging at 4,000 g for 8 min. The following steps were performed on ice using buffers that had been sterile-filtered. The supernatant was discarded, and the pellets were fully resuspended in 40 ml TBS buffer (20 mM Tris-HCl pH 7.5, 0.15 M NaCl). After centrifuging at 4,000 g for 8 min at 4° C., the supernatant was removed, and the pellet was resuspended in 40 ml TBS buffer again. Next, the OD600 was measured for a 1:1 mixture of the cell suspension and fresh TBS buffer, and a standardized volume equivalent to 40 ml of OD600=0.6 was aliquoted into new 50 ml conical tubes. A final 8 min centrifugation step at 4,000 g and 4° C. was performed, cells were pelleted and the supernatant was discarded. Residual liquid was removed, and cell pellets were flash-frozen using liquid nitrogen and stored at −80° C. or kept on ice for the subsequent steps.

Bovine serum albumin (GoldBio) was dissolved in 1×PBS buffer (Gibco) and sterile-filtered to generate a 5 mg ml-1 BSA solution. For each sample, 25 μl of Dynabeads Protein G (Thermo Fisher Scientific) slurry (hereafter, beads or magnetic beads) were prepared for immunoprecipitation. Up to 250 μl of the initial bead slurry were prepared in a single tube, and washes were performed at room temperature, as follows: the slurry was transferred to a 1.5 ml tube and placed onto a magnetic rack. The supernatant was removed, 1 ml BSA solution was added, and the beads were fully resuspended by vortexing, followed by rotating for 30 s. This was repeated for three more washes. Finally, the beads were resuspended in 25 μl (×n samples) of BSA solution, followed by addition of 4 μl (×n samples) of monoclonal anti-Flag M2 antibodies produced in mouse (Sigma-Aldrich). The suspension was moved to 4° C. and rotated for >3 h to conjugate antibodies to magnetic beads. While conjugation was proceeding, cross-linked cell pellets were thawed on ice, resuspended in FA lysis buffer 150 (50 mM HEPES-KOH pH 7.5, 0.1% (w/v) sodium deoxycholate, 0.1% (w/v) SDS, 1 mM EDTA, 1% (v/v) Triton X-100, 150 mM NaCl) with protease inhibitor cocktail (Sigma-Aldrich) and transferred to a 1 ml milliTUBE AFA Fiber (Covaris). The samples were sonicated on a M220 Focused-ultrasonicator (Covaris) with the following SonoLab 7.2 settings: minimum temperature, 4° C.; set point, 6° C.; maximum temperature, 8° C.; peak power, 75.0; duty factor, 10; cycles/bursts, 200; 17.5 min sonication time. After sonication, samples were cleared of cell debris by centrifugation at 20,000 g and 4° C. for 20 min. The pellet was discarded, and the supernatant (˜1 ml) was transferred into a fresh tube and kept on ice for immunoprecipitation. For non-immunoprecipitated input control samples, 10 μl (˜1%) of the sheared cleared lysate were transferred into a separate 1.5 ml tube, flash-frozen in liquid nitrogen and stored at −80° C.

After greater than 3 h, the conjugation mixture of magnetic beads and antibodies was washed four times with BSA solution as described above, but at 4° C. Next, the beads were resuspended in 30 μl (× n samples) FA lysis buffer 150 with protease inhibitor, and 31 μl of resuspended antibody-conjugated beads were mixed with each sample of sheared cell lysate. The samples rotated overnight for 12-16 h at 4° C. for immunoprecipitation of Flag-tagged proteins. The next day, tubes containing beads were placed on a magnetic rack, and the supernatant was discarded. Then, six bead washes were performed at room temperature, as follows, using 1 ml of each buffer followed by sample rotation for 1.5 min: (1) two washes with FA lysis buffer 150 (without protease inhibitor); (2) one wash with FA lysis buffer 500 (50 mM HEPES-KOH PH 7.5, 0.1% (w/v) sodium deoxycholate, 0.1% (w/v) SDS, 1 mM EDTA, 1% (v/v) Triton X-100, 500 mM NaCl); (3) one wash with ChIP wash buffer (10 mM Tris-HCl pH 8.0, 250 mM LiCl, 0.5% (w/v) sodium deoxycholate, 0.1% (w/v) SDS, 1 mM EDTA, 1% (v/v) Triton X-100, 500 mM NaCl); and (4) two washes with TE buffer 10/1 (10 mM Tris-HCl pH 8.0, 1 mM EDTA). The beads were then placed onto a magnetic rack, the supernatant was removed, and the beads were resuspended in 200 μl of fresh ChIP elution buffer (1% (w/v) SDS, 0.1 M NaHCO₃). To release protein-DNA complexes from beads, the suspensions were incubated at 65° C. for 1.25 h with gentle vortexing every 15 min to resuspend settled beads. During this incubation, the non-immunoprecipitated input samples were thawed, and 190 μl of ChIP Elution Buffer was added, followed by the addition of 10 μl of 5 M NaCl. After the 1.25 h incubation of the immunoprecipitated samples was complete, the tubes were placed back onto a magnetic rack, and the supernatant containing eluted protein-DNA complexes was transferred to a new tube. Then, 9.75 μl of 5 M NaCl was added to ˜195 μl of eluate, and the samples (both immunoprecipitated and non-immunoprecipitated controls) were incubated at 65° C. overnight to reverse-cross-link proteins and DNA. The next day, samples were mixed with 1 μl of 10 mg ml 1 RNase A (Thermo Fisher Scientific) and incubated for 1 h at 37° C., followed by addition of 2.8 μl of 20 mg ml 1 proteinase K (Thermo Fisher Scientific) and 1 h incubation at 55° C. After adding 1 ml of buffer PB (QIAGEN recipe), the samples were purified using QIAquick spin columns (QIAGEN) and eluted in 40 μl TE buffer 10/0.1 (10 mM Tris-HCl pH 8.0, 0.1 mM EDTA).

ChIP-seq Illumina libraries were generated for immunoprecipitated and input samples using the NEBNext Ultra II DNA Library Prep Kit for Illumina (NEB). Sample concentrations were determined using the DeNovix dsDNA Ultra High Sensitivity Kit. Starting DNA amounts were standardized such that an approximately equal mass of all input and immunoprecipitated DNA was used for library preparation. After adapter ligation, PCR amplification (12 cycles) was performed to add Illumina barcodes, and ˜450 bp DNA fragments were selected using two-sided AMPure XP bead (Beckman Coulter) size selection, as follows: the volume of barcoded immunoprecipitated and input DNA was brought up to 50 μl with TE Buffer 10/0.1; in the first size-selection step, 0.55×AMPure beads (27.5 μl) were added to the DNA, the sample was placed onto a magnetic rack, and the supernatant was discarded and the AMPure beads were retained; in the second size-selection step, 0.35×AMPure beads (17.5 μl) were added to the DNA, the sample was placed onto a magnetic rack, and the AMPure beads were discarded and the supernatant was retained. The concentration of DNA was determined for pooling using the DeNovix dsDNA High Sensitivity Kit.

Illumina libraries were sequenced in paired-end mode on the Illumina MiniSeq and NextSeq platforms with automated demultiplexing and adapter trimming (Illumina). For each ChIP-seq sample, >1,000,000 raw reads (including genomic and plasmid-mapping reads) were obtained.

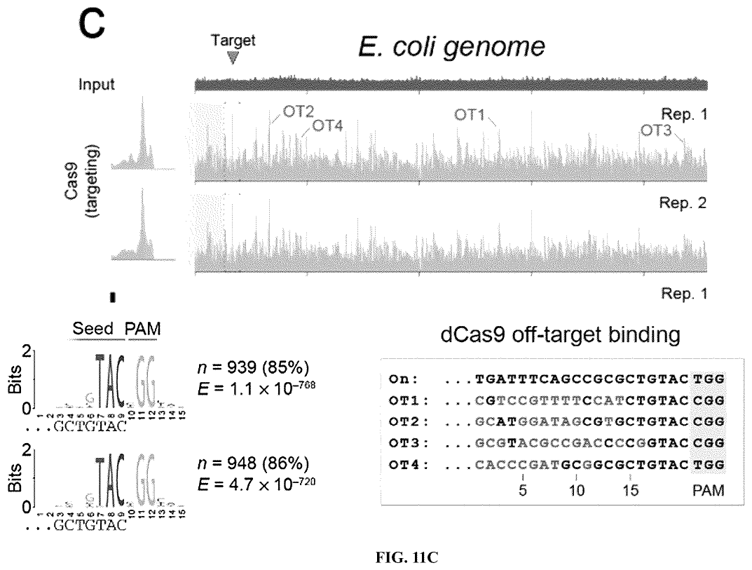

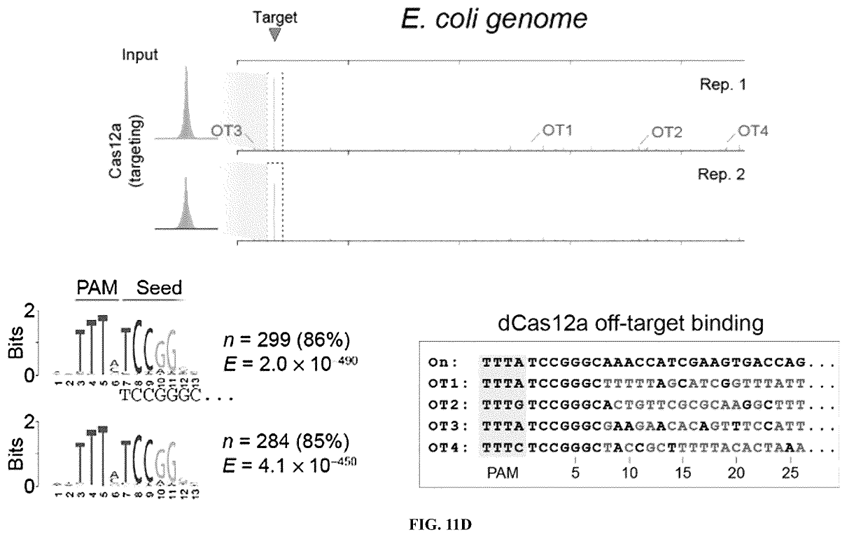

ChIP-seq data analyses. ChIP-seq data analysis was generally performed as described previously (Sec, Hoffmann, F. T. et al., Nature 609, 384-393 (2022), incorporated herein by reference). In brief, ChIP-seq paired-end reads were trimmed and mapped to an E. coli BL21 (DE3) reference genome (GenBank: CP001509.3). Genomic lacZ and lacI regions, partially identical to plasmid-encoded genes, were masked in all alignments (genomic coordinates: 335,600-337,101 and 748,601-750,390). In the ChIP-seq analysis of Cas9 and Cas12a, rrnB t1 terminator genomic sequence was masked (genomic coordinates: 4,121,275-4,121,400). Mapped reads were sorted, indexed, and multi-mapping reads were excluded. Aligned reads were normalized by RPKM and visualized in IGV. For genome-wide views, maximum read coverage values were plotted in 1-kb bins. Peak calling was performed using MACS3 with respect to non-immunoprecipitated control samples of TnpB and Cas9. The peak summit coordinates in the MACS3 output summits.bed file were extended to encompass a 200-bp window using BEDTools. The corresponding 200-bp sequence for each peak was extracted from the E. coli reference genome using the command bedtools getfasta. Sequence motifs were determined using MEME ChIP. Individual off-target sequences (FIG. 11) represent sequences from the top enriched peaks determined by MACS3 that contain the MEME ChIP motif.

TAM library cloning. TAM libraries were cloned containing a 6-bp randomized sequence between the native target sequences for GstIscB (ISGst5) and GstTnpB2 (ISGst2). In brief, two partially overlapping oligos (oSL9404 and oSL9405) were annealed by heating to 95° C. for 2 min and then cooled to room temperature. One of these oligos (oSL9404) contained a 6-nt degenerate sequence flanked by target sites for GstTnpB2 and GstIscB. Annealed DNA was treated with DNA Polymerase I, Large (Klenow) Fragment (NEB) in 40 μL reactions and incubated at 37° C. for 30 min, then gel purified (QIAGEN Gel Extraction Kit). Double-stranded insert DNA and vector backbone (pSL4031) was digested with BamHI and HindIII (37° C., 1 h). The digested insert was cleaned-up (Qiagen MinElute PCR Purification Kit), and digested backbone was gel-purified (Qiagen QIAquick Gel Extraction Kit). The backbone and insert were ligated with T4 DNA Ligase (NEB). Ligation reactions were transformed in with electrocompetent NEB 10-beta cells according to the manufacturer's protocol. After recovery (37° C. for 1 h), cells were plated on large bioassay plates containing LB agar and kanamycin (50 μg ml⁻¹). Approximately 5 million CFUs were scraped from each plate, representing 1000× coverage of each library member, and plasmid DNA was isolated using the Qiagen CompactPrep Midi Kit.

TAM library assays and NGS library prep. DNA solutions containing 500 ng of the TAM plasmid library (pSL4841) and 500 ng of plasmids encoding either GstTnpB2 (pSL4369) or GstIscB (pSL4514) were co-transformed in electrocompetent E. coli BL21 (DE3) cells according to the manufacturer's protocol (Sigma-Aldrich). Cells were serially diluted on large bioassay plates containing LB agar, spectinomycin (100 μg ml⁻¹), and kanamycin (50 μg ml⁻¹). Approximately 600,000 CFUs were scraped from plates, representing 100× coverage of each library member, and plasmid DNA was isolated using the Qiagen CompactPrep Midi Kit. Illumina amplicon library for NGS was prepared through 2-step PCR amplification. In brief, ˜50 ng of plasmid DNA recovered from TAM assay was used in each “PCR-1” amplification reaction with primers flanking the degenerate TAM library sequence and containing universal Illumina adaptors as 5′ overhangs. Amplification was carried out using high-Fidelity Q5 DNA Polymerase (NEB) for 16 thermal cycles. Samples from “PCR-1” amplification were diluted 20-fold and amplified for “PCR-2” in 10 thermal cycles with primers contain indexed p5/p7 sequences. Reactions were verified by analytical gel electrophoresis. Sequencing was performed with a paired-end run using a MiniSeq High Output Kit with 150-cycles (Illumina).

Analyses of NGS TAM library data. Analysis of TAM depletion library was performed using a custom Python script. Demultiplexed reads were filtered to remove reads that did not contain a perfect match to the 58-bp sequence upstream of the degenerate sequence for any i5-reads. For reads that passed this filtering step, the 6-nt degenerate sequence was extracted and counted. The relative abundance of each degenerate sequence in a sample was determined by dividing the degenerate sequence count by the total number of sequence counts for that sample. Then, the fold-change between the output and input libraries was calculated by dividing the relative abundance of each degenerate sequence in the output library by its relative abundance in the input library, and then log 2-transformed. Sequence logos were constructed by taking the 10 most depleted sequences and generated using WebLogo (v2.8).

Transposition assays combining TnpA and TnpB. E. coli str. K-12 substr. MG1655 (sSL0810) was engineered to carry a genomic integrated mini-Tn containing a kanamycin resistance cassette inserted into lacZ by recombineering as described above to generate sSL2771. This strain was transformed with either pCDFDuct-1 (pSL0007) or various GstTnpB carrying vectors (pSL4369, pSL4664, pSL4518 and pSL4740, see Table 8 for description) and selected on LB agar containing spectinomycin (100 μg ml⁻¹) and kanamycin (50 μg ml⁻¹). Single colony isolates of cells harboring each plasmid were prepared chemically competent and transformed with a TnpA expression vector (pSL4529) or a catalytically inactive mutant TnpA expression vector (pSL4534) and selected on LB agar containing carbenicillin (100 μg ml⁻¹), spectinomycin (100 μg ml⁻¹) and kanamycin (50 μg ml⁻¹). Three single colony isolate of each transformant were grown in liquid LB containing carbenicillin (100 μg ml⁻¹), spectinomycin (100 μg ml⁻¹) and kanamycin (50 μg ml⁻¹) and grown for 14 h at 37° C. Optical density (OD) of each culture was measured and approximately 10⁷ cells were plated onto MacConkey agar media containing carbenicillin (100 μg ml⁻¹), spectinomycin (100 μg ml⁻¹) and 0.05 mM IPTG for TnpA induction. Importantly, the media did not contain kanamycin to allow for excision of the mini-Tn. Cells were grown at 37° C. for 4 days on MacConkey media to enrich for mini-Tn excision events. Cells were then harvested, serially diluted, and plated onto LB agar containing carbenicillin (100 μg ml⁻¹), spectinomycin (100 μg ml⁻¹) and X-gal (200 mg ml 1) or carbenicillin (100 μg ml⁻¹), spectinomycin (100 μg ml⁻¹), kanamycin (50 μg ml⁻¹) and X-gal (200 mg ml 1) and grown for 18 h at 37° C. Total number of colonies were counted, along with the number of blue colonies to determine the frequency of excision and reintegration events. In addition, genomic lysate was harvested from cells as described above for PCR analysis.

Statistics and reproducibility. qPCR and analytical PCRs resolved by agarose gel electrophoresis gave similar results in three independent replicates. Sanger sequencing of excision products was performed once for each isolate. Next-generation sequencing of PCR amplicons was performed once. Plasmid interference assays were performed in three independent replicates. Transposition assays combining TnpA and TnpB were performed with three independent replicates.

Data availability. Next-generation sequencing data are available in the National Center for Biotechnology Information (NCBI) Sequence Read Archive: SRX19058888-SRX19058905, SRR23476356-SRR23476358 (BioProject Accession: PRJNA925099) and the Gene Expression Omnibus (GSE223127). The published genome used for ChIP-seq analyses was obtained from NCBI (GenBank: CP001509.3). The published genome used for bioinformatics analyses of the Geobacillus stearothermophilus genome was obtained from NCBI (GenBank: NZ_CP016552.1).

Code availability. Custom scripts used for bioinformatics, TAM library analyses, and ChIP-seq data analyses are available at GitHub (github.com/sternberglab/Meers_et_al_2023).

### Example 1

### G. Stearothermophilus Encodes Diverse TnpB/IscB Homologs

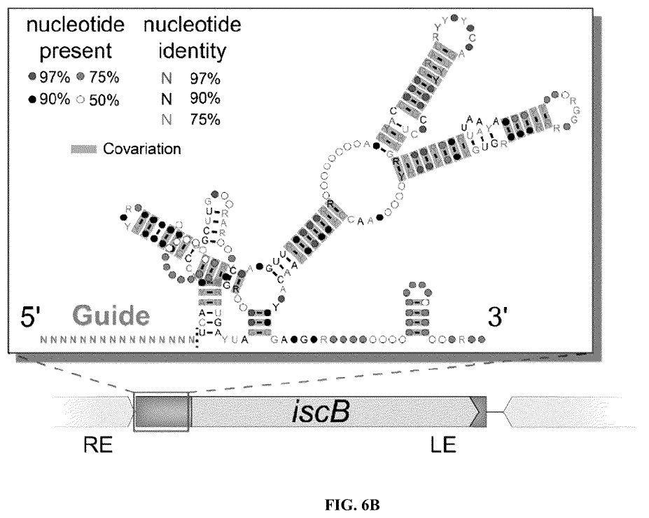

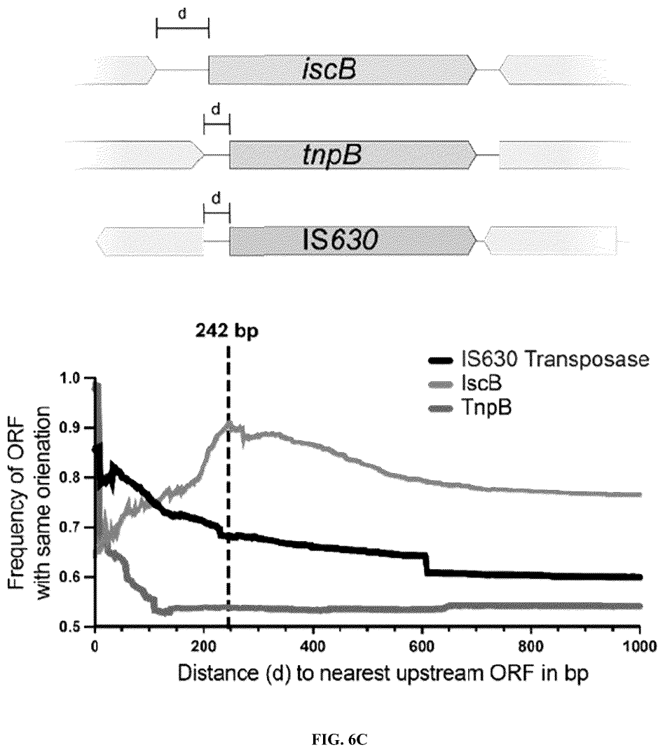

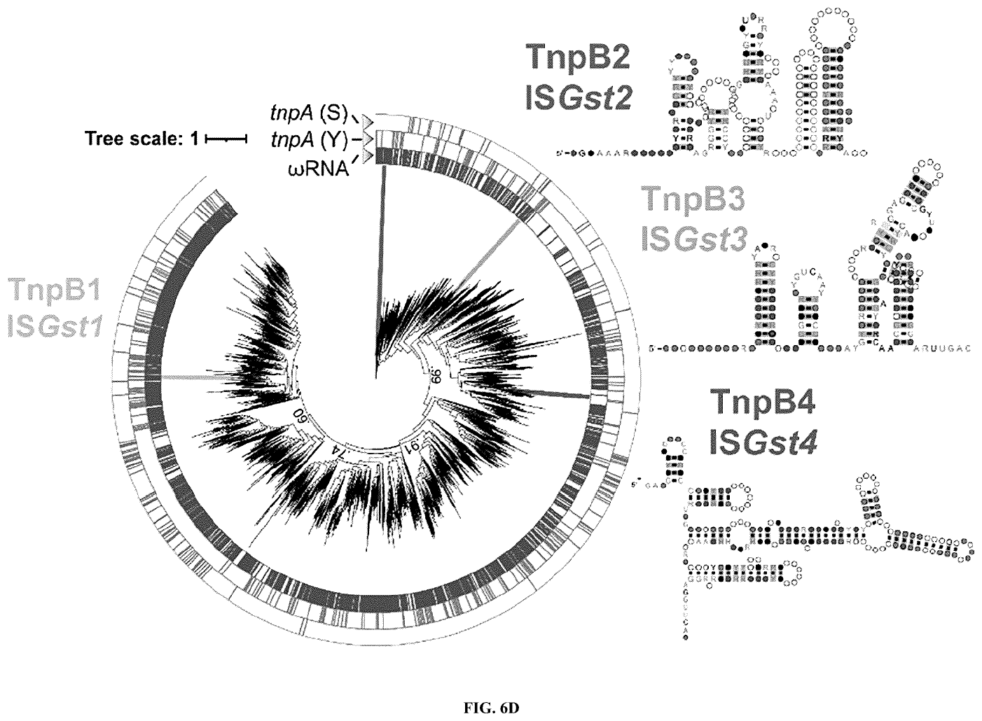

The NCBI NR database was mined for TnpB/IscB homologs and phylogenetic trees were built that highlight the diversity of both protein families (FIGS. 6A and 6D). When extracting flanking genomic regions, only a sporadic association with Y1 tyrosine transposases was identified, with ˜25% of all tnpB genes containing an identifiable tnpA nearby, indicative of autonomous transposons. Interestingly, iscB genes were much less abundant than tnpB and rarely associated with tnpA (˜1.5%). This suggested that the vast majority of tnpB/iscB genes are encoded within transposons lacking tnpA, suggesting a non-autonomous function that would indicate transposases encoded elsewhere mobilize them in trans (FIGS. 6A and 6D). TnpB but not iscB genes were also found associated with an unrelated serine resolvase (also denoted tnpA) that is a hallmark of IS607-family transposons, albeit at a much lower frequency (˜8%) (FIG. 6D).

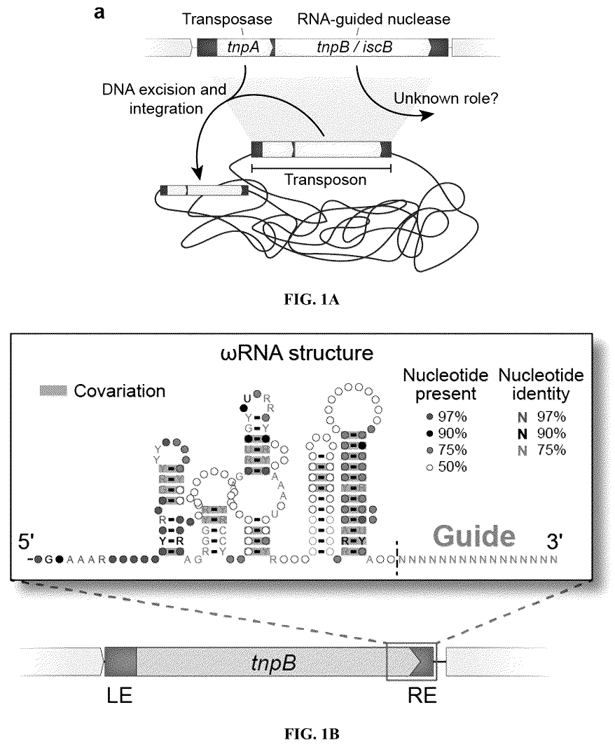

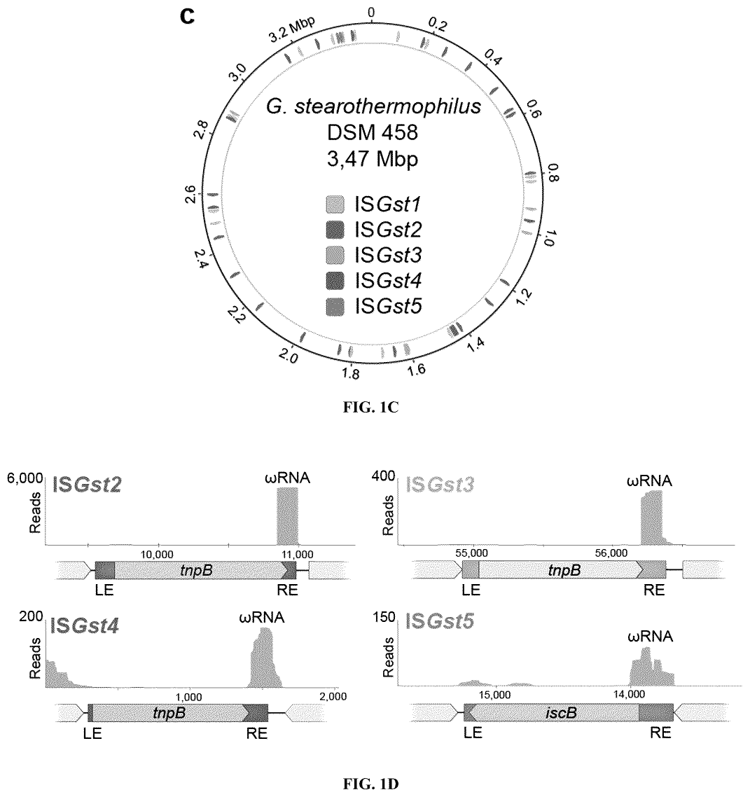

A conserved intergenic region upstream of iscB was bounded by the transposon right end (RE), and bore similarity to a non-coding RNAs. Both IscB and TnpB use these transposon-encoded RNAs, referred to hereafter as ωRNAs, as guides to direct cleavage of complementary dsDNA substrates, in a mechanism analogous to Cas9 and Cas12. Covariation models were generated for TnpB- and IscB-specific ωRNAs, which revealed the conserved secondary structural motifs characteristic of both guide RNAs (FIGS. 1B and 6B), and these models were used to demonstrate the tight genetic linkage between tnpB/iscB genes and flanking ωRNA loci (FIGS. 6A and 6D). In order to investigate whether ωRNA production might be sensitive to local genetic context, the orientation of genes upstream of iscB were analyzed throughout the diverse members in the phylogenetic tree and a strong bias for genes encoded in the same orientation was observed (FIG. 6C). Since IscB-specific ωRNAs comprise a constant scaffold sequence derived from the transposon RE, joined by a 5′-adjacent guide region encoded outside of the transposon boundary, ωRNA biogenesis relies on transcription initiating outside of the IS element and proceeding towards the iscB ORF (FIG. 6B). Genomic insertions into transcriptionally active target sites may aid in the generation of functional ωRNAs, and these insertion products are either preferentially generated (during transposition) or preferentially retained. Notably, this orientation bias was absent for TnpB, whose ωRNA substrates rely on transcription that initiates within the IS element itself (described below), and for an unrelated IS630-family transposase that were included as a negative control (FIG. 6C).

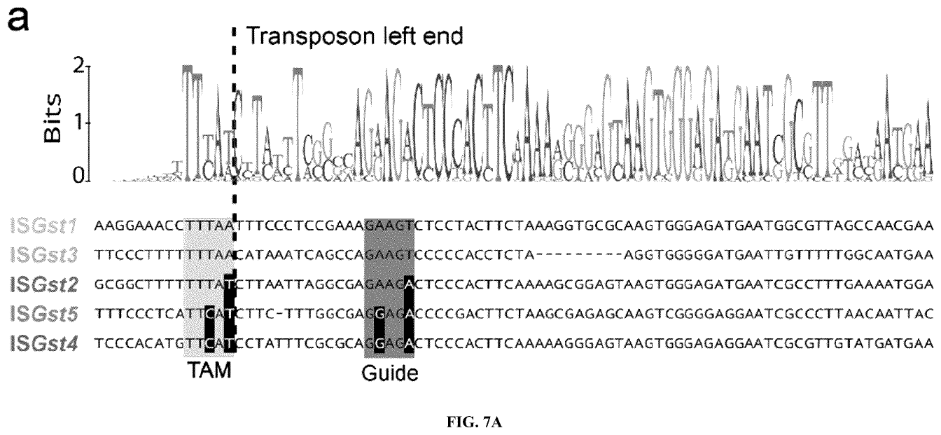

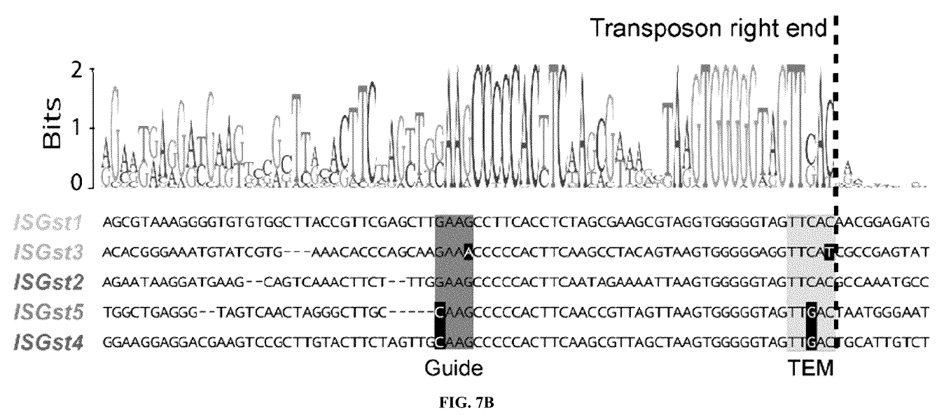

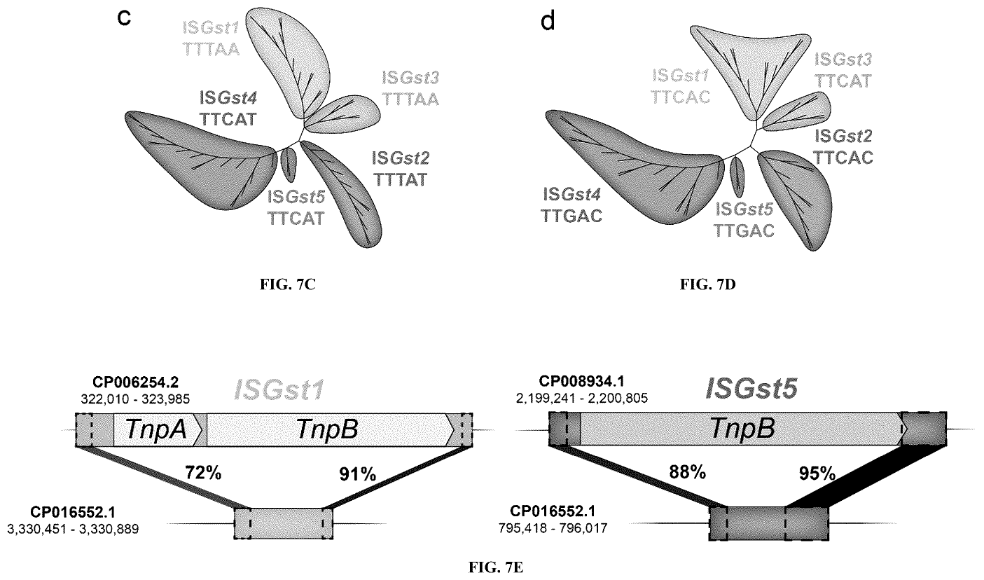

Geobacillus stearothermophilus (Gst), a thermophilic soil bacterium, has a substantial expansion of five IS605-family elements encoding both TnpB and IscB, denoted ISGst1-5, collectively comprising ˜ 1% of the entire genome (FIG. 1C). Analysis of small RNA sequencing data revealed that ωRNAs from multiple transposons were constitutively expressed (FIG. 1D), and the left end (LE) and right end (RE) boundaries of these IS elements were highly similar in DNA sequence (FIGS. 7A-7D), suggesting a common mechanism of mobilization. Using this information, a candidate tnpA gene responsible for transposing these elements was identified, as well as minimal non-autonomous IS elements that lacked protein-coding genes altogether and resembled palindrome-associated transposable elements (PATEs; FIG. 7E).

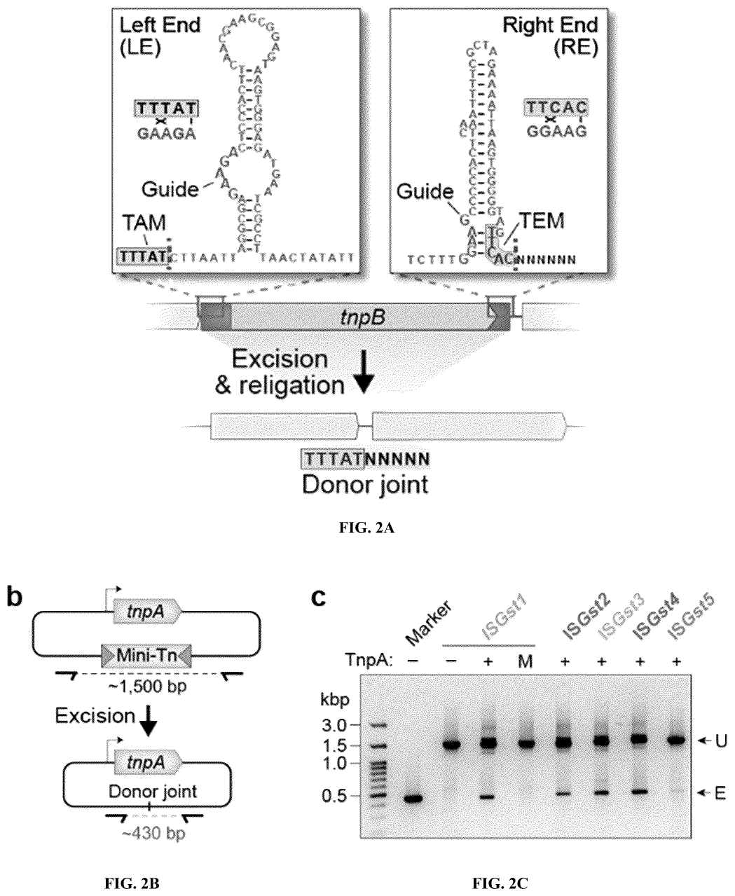

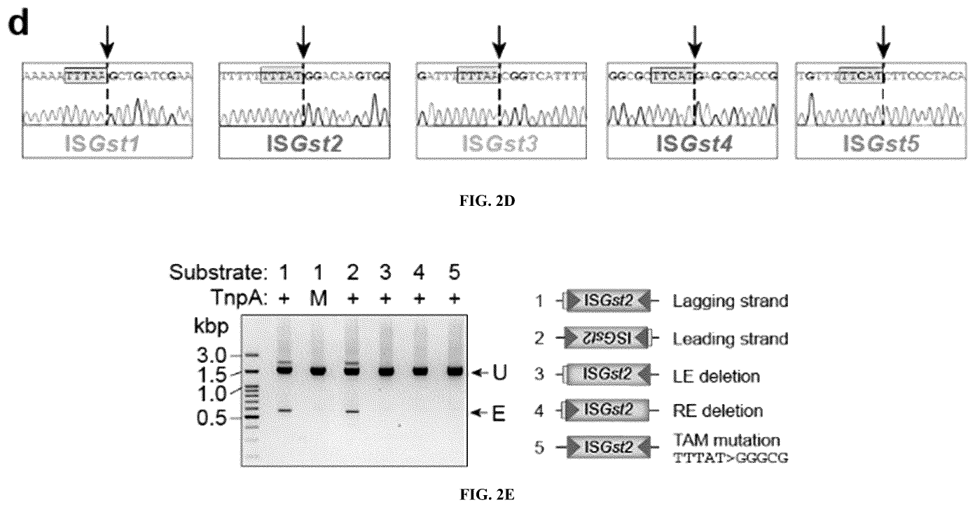

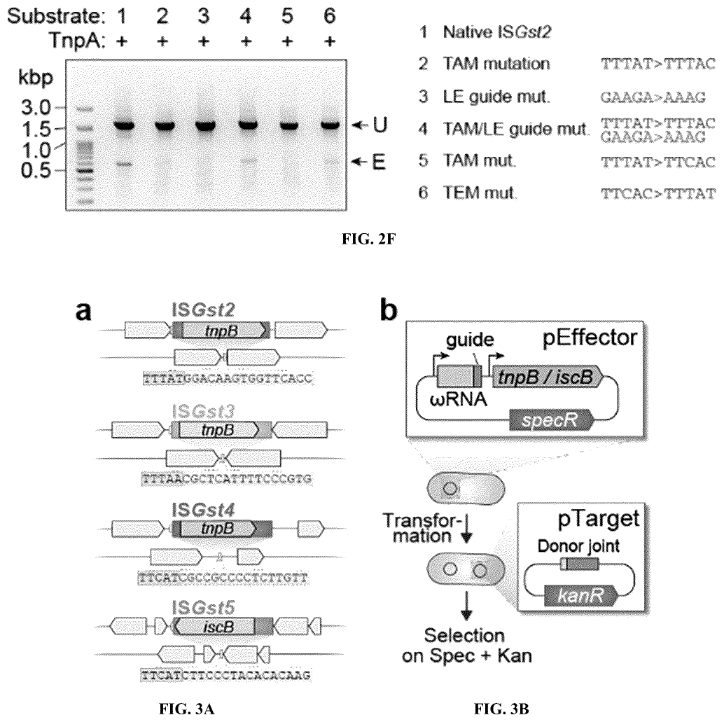

Interestingly, in addition to sharing similar sequences within the LE and RE, ISGst1-5 elements exhibited conserved, clade-specific transposon-adjacent motifs and transposon-encoded motifs (TAMs and TEMs; FIGS. 7A-D). Prior studies on the TnpA transposase from Helicobacter pylori IS608, which transposes a related IS605-like element, revealed that these motifs constitute the target and cleavage sites recognized during transposon insertion and transposon excision reactions, respectively. Yet rather than being recognized exclusively through protein-DNA recognition, these motifs form non-canonical base-pairing interactions with a DNA ‘guide’ sequence located in the sub-terminal ends of the IS element (FIG. 2A). Focusing on multiple sequence alignments between ISGst1-5 elements, covarying mutations between both the TAM/TEM sequences and their associated DNA guide sequences were observed (FIGS. 2A, 7A and 7B).

### Example 2

### GstTnpA is Active for DNA Excision and Transposition

A DNA excision assay was designed to test the activity of GstTnpA on a mini-transposon (mini-Tn) substrate derived from its native autonomous IS element, ISGst1. E. coli expression vectors that encoded GstTnpA upstream of the mini-Tn, which comprised an antibiotic resistance gene flanked by full-length LE and RE sequences and genomic G. stearothermophilus sequences upstream and downstream of the predicted transposon boundaries were cloned. Primers were designed to bind outside the mini-Tn, such that PCR from cellular lysates would amplify either the starting substrate or a shorter reaction product resulting from transposon excision and re-ligation (FIGS. 2A-2B). A parallel panel of substrates containing LE and RE sequences derived from ISGst2-5, which natively encode IscB, TnpB, or ωRNA only, were generated to determine the breadth of GstTnpA substrate recognition. Remarkably, GstTnpA was active on all five families of IS elements, with excision dependent on the predicted catalytic tyrosine residue (FIG. 2C), but failed to cross-react with a DNA substrate derived from an H. pylori IS608 element (FIGS. 8A-8B). Sanger sequencing of excision products revealed that in each case, TnpA precisely re-joined sequences flanking the mini-Tn to generate a scarless donor joint (FIG. 2C), which could be recognized and cleaved by TnpB/IscB (see below). Using an alternative qPCR-based strategy to prime directly off the donor joint sequence, excision frequencies of 0.70% were calculated directly from overnight cultures (FIGS. 8C-8E).

Excision proceeded regardless of whether the mini-Tn was encoded on the leading or lagging-strand template, but was ablated when either the LE or RE sequence were scrambled, confirming the importance of these regions for TnpA recognition. Excision was also strongly dependent on the presence of a cognate TAM adjacent to the LE as well as a compatible DNA ‘guide’ sequence located within the LE, since mutation of either region led to a loss of product formation (FIG. 2E). Interestingly, however, simultaneous mutation of both the TAM and LE guide sequence to the corresponding motifs found in IS608 restored excision activity with GstTnpA (FIG. 2F). Similar base-pairing interactions occur between a DNA ‘guide’ sequence within the RE and a matching TEM found at the RE boundary, with only minor differences between the TAM and TEM at positions 3 and 5 (FIGS. 8A and 8B). Whereas the excision reaction did not tolerate mutation of the TAM sequence to the TEM sequence, mutations to the TEM were still tolerated, despite ablating predicted base-pairing interactions with the RE ‘guide’ sequence (FIG. 2F). However, closer inspection revealed that these excision events resulted from erroneous selection of an alternative mini-Tn boundary downstream of the native RE, at a sequence matching the WT TEM (TTCAC; FIGS. 8F-8G). These results indicated that IS200/IS605-family elements tolerate flexible spacing between the TAM/TEM and corresponding guide sequences, allowing for capture of additional sequences outside of the native LE and RE boundaries.

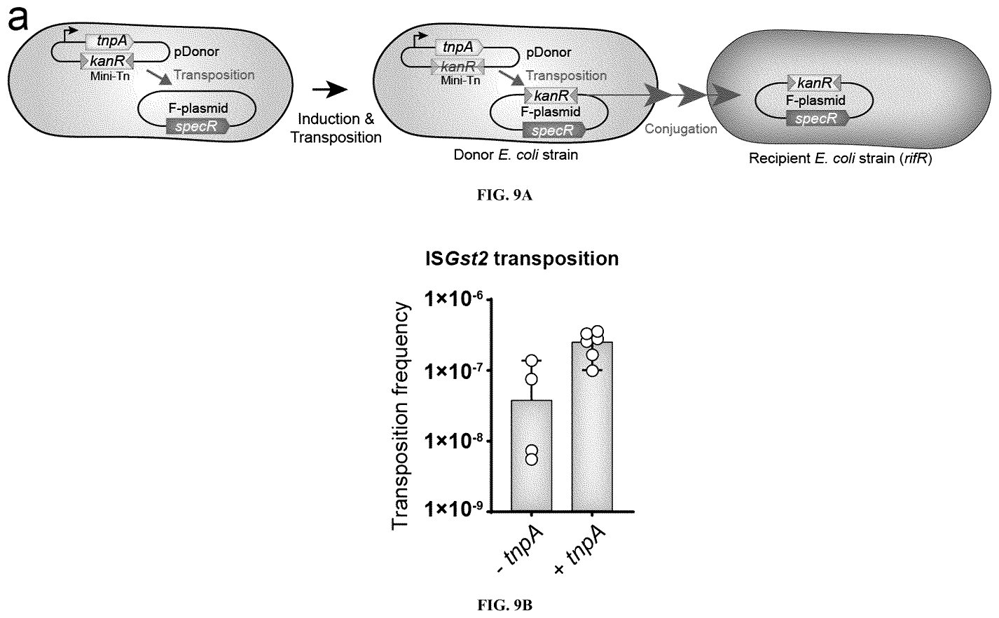

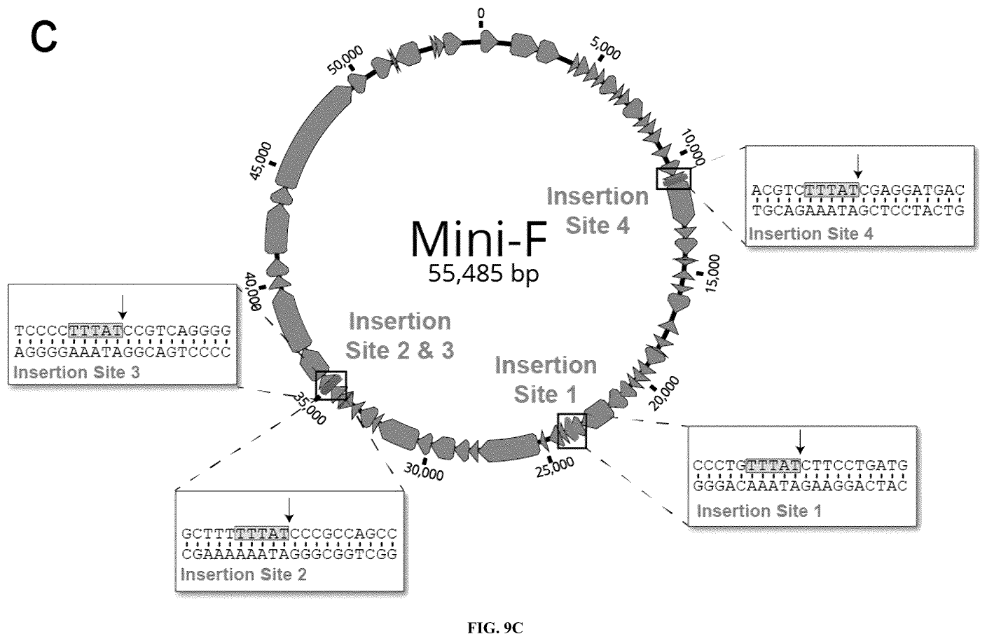

Using a traditional mating-out assay with the ISGst2 mini-Tn (FIG. 9A), in which transposition events into a conjugative plasmid are isolated via drug selection, transposition efficiencies of 2.5×10−7 were measured, which were several orders of magnitude lower than the observed rates of transposon excision (FIGS. 8E and 9B). These results suggest that, under the tested experimental conditions, TnpA expression would eventually lead to permanent transposon loss from the cell population, absent any active mechanisms for maintaining transposons at their donor sites during or after excision (see below). Long-read sequencing of drug-resistant transconjugants confirmed the presence of novel mini-Tn insertions, which were invariably located downstream of endogenous TAM sites on the F-plasmid (FIG. 9C). Collectively, these experiments demonstrated that GstTnpA is active in mobilizing a large network of diverse, IS605-like elements found in the G. stearothermophilus genome, but that its intrinsic enzymatic properties render transposons vulnerable to being permanently lost from the population without an active mechanism for donor-site preservation.

### Example 3

### GstTnpB and IscB Homologs Function as RNA-Guided Endonucleases

With knowledge that GstTnpA was active in mobilizing diverse IS elements, nuclease activity for the associated GstTnpB/IscB proteins was tested using a plasmid interference assay, in which successful targeting leads to plasmid cleavage and a loss of cellular viability (FIG. 3B). Expression plasmids encoding both TnpB/IscB and the corresponding ωRNA guides derived from their native GstIS elements (pEffector) were designed, alongside target plasmids containing donor joints that were bioinformatically identified and experimentally verified in TnpA excision assays (pTarget; FIGS. 2D and 3A). After screening various promoter combinations driving expression of the nuclease and ωRNA (FIG. 10A), GstIscB and three GstTnpB distinct homologs were highly active for RNA-guided DNA cleavage of their native donor joints (FIGS. 3C and 3D). Interestingly, HpyTnpB encoded by the well-studied IS608 element was inactive when tested under similar conditions, whereas the activity for DraTnpB was confirmed (FIG. 10B).

The TAM on pTarget was systematically mutagenized and DNA cleavage was ablated with even single-bp changes, which would also render the site of ωRNA biogenesis at the transposon RE, where the motif differs from the cognate TAM in only two positions, completely unrecognizable (FIGS. 3E-3F and 10C-10D). TnpB and IscB were both functional for genomic targeting and cleavage as well, and point mutations in the predicted HNH and/or RuvC nuclease domains completely ablated activity (FIGS. 3G and 3H). Interestingly, a panel of three TnpB-specific ωRNAs targeting lacZ showed varying levels of activity, as assessed by cell lethality (FIG. 10E).

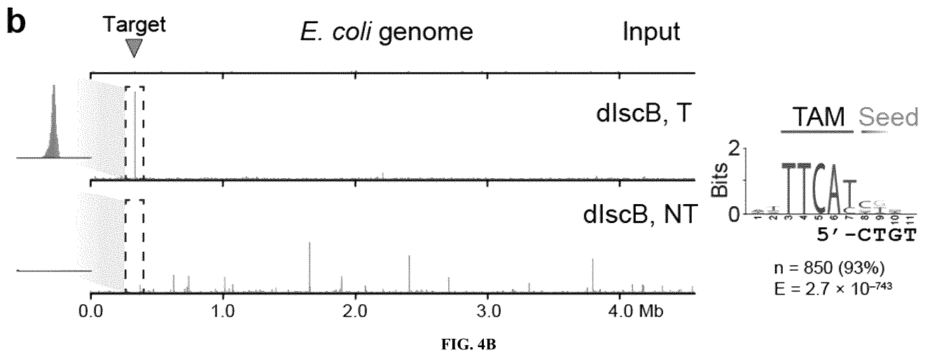

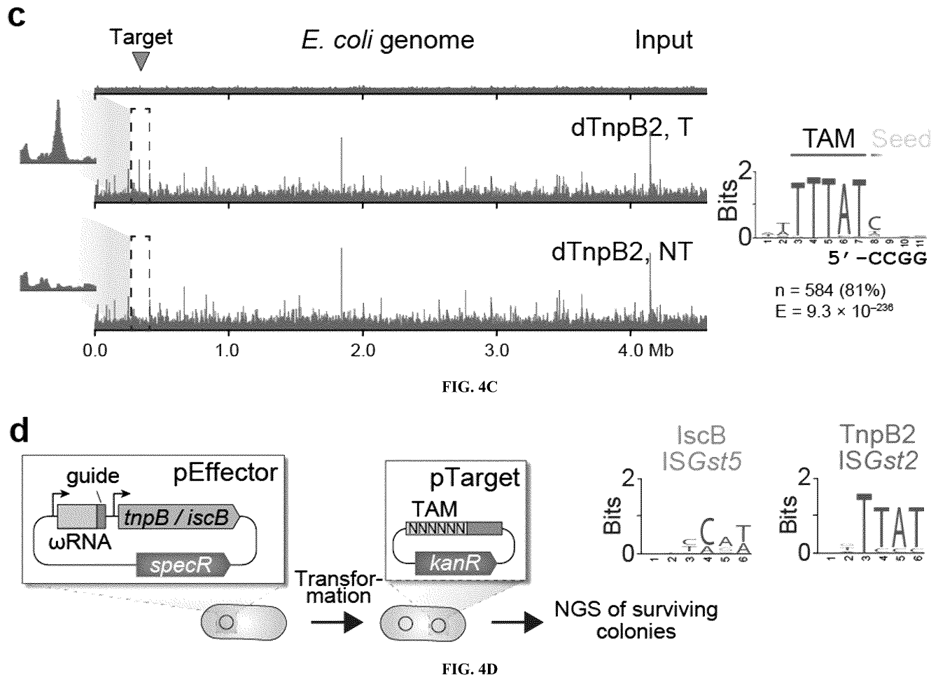

To investigate binding specificity in more detail, ChIP-seq experiments were performed to map all chromosomal binding sites of nuclease-dead IscB and TnpB programmed with lacZ-specific ωRNAs (FIG. 4A). The resulting data revealed strong enrichment at the on-target site and numerous off-targets (FIGS. 11A-11D), and the majority of peaks shared highly conserved consensus motifs of 5′-TTCAT-3′ (IscB from ISGst5) and 5′-TTTAT-3′ (TnpB2 from ISGst2) (FIGS. 4B-4C), which precisely matched the TAM motifs neighboring the native ISGst5 and ISGst2 elements, respectively (FIG. 7A). Similar consensus motifs emerged when cleavage activity in cells was tested using pTarget libraries containing degenerate TAM sequences (FIG. 4D), indicating common sequence determinants for DNA target binding and cleavage. Neither IscB nor TnpB exhibited a strong requirement for extensive complementarity within the seed sequence for the off-target sites analyzed (FIGS. 11A-11B), and this absence was particularly striking in comparison to matched experiments with Cas9 and Cas12a, which were strongly dependent on 3-5 nt of PAM-adjacent sequence matching the guide RNA (FIGS. 11C-11D). Cas9 and Cas12 may have evolved a greater degree of reliance on RNA-DNA complementarity for stable DNA binding, whereas IscB and TnpB may be dependent on a more extensive TAM motif but permissive of RNA-DNA mismatches.

### Example 4

### RNA-Guided Nucleases Promote Transposon Retention Through Targeted DSBs

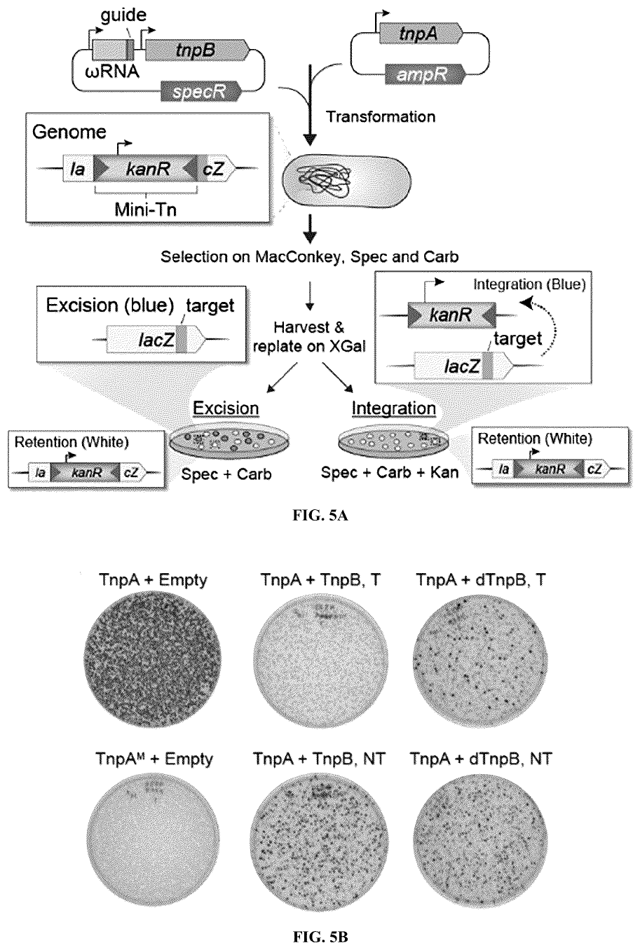

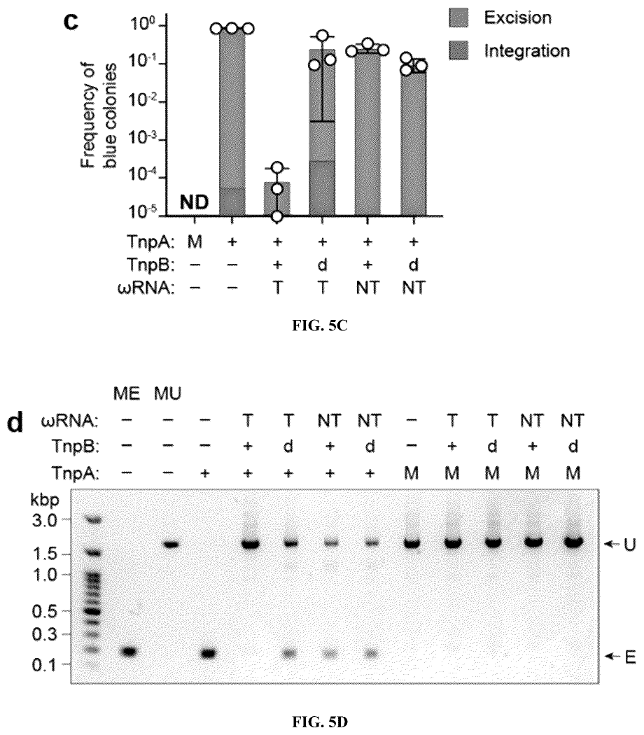

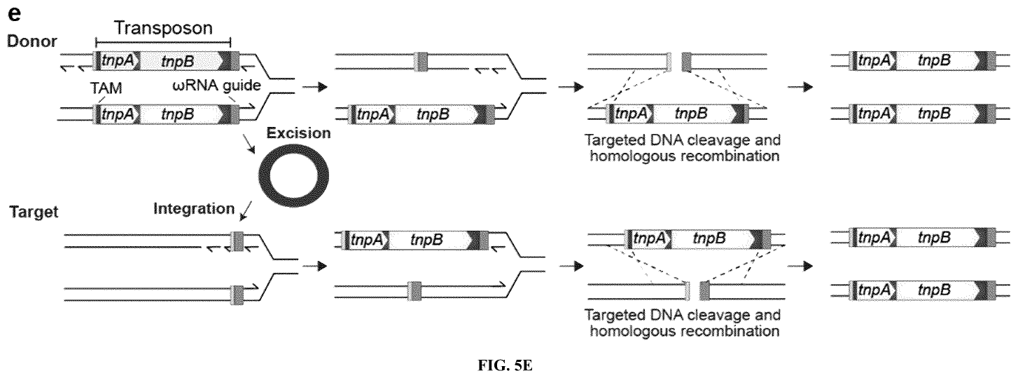

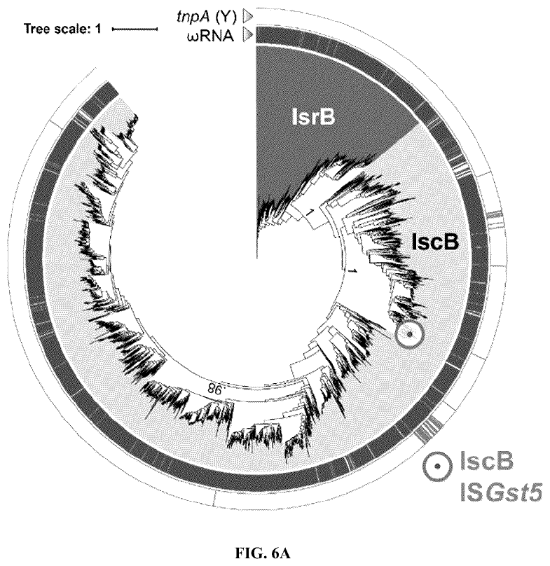

To test if IscB/TnpB nucleases with compatible ωRNAs would rapidly intercept the donor joint products generated upon transposon excision by TnpA and promote reinstallation of transposon copies at pre-existing donor sites, an E. coli strain harboring a lacZ-interrupting mini-Tn that was inserted downstream of a TnpB-compatible TAM was generated, such that scarless excision by TnpA would result in a phenotypic switch from lacZ (white colony phenotype) to functional lacZ+ (blue colony phenotype; FIG. 5A). Strains were transformed with expression plasmids encoding TnpA (or an inactive mutant) and TnpB (or an inactive mutant), programmed with either a non-targeting ωRNA or a lacZ-targeting ωRNA designed to cleave the donor joint generated upon TnpA-mediated mini-Tn excision. After enriching for excision events by growing strains on MacConkey agar, cells were plated on media containing X-gal and performed blue-white colony screening. Using this approach, the emergence of a large fraction of blue colonies were observed in the presence of WT TnpA, but not a catalytically inactive mutant, and colony PCR analysis confirmed that these colonies had indeed permanently lost the transposon at the donor lacZ locus (FIGS. 5B-5D). When a similar population of cells was plated onto X-gal plates that also contained kanamycin, thus selecting for the presence of the mini-Tn, blue colonies were 1000× less abundant (FIG. 5C), confirming that the frequency of transposon excision at the donor site vastly exceeds the frequency of transposon integration at a new target site.

Remarkably, co-expression of TnpB and a lacZ-specific ωRNA completely eliminated the emergence of blue colonies under otherwise identical conditions, and colony PCR confirmed that transposons were uniformly maintained at their original genomic location (FIGS. 5B-5D and 12). This phenotypic effect was dependent on both a targeting ωRNA and an intact TnpB nuclease domain, indicating that targeting/binding alone is insufficient for transposon retention at the donor site, but that targeted cleavage and local DSB formation facilitate the effect. TnpB nucleases preserve transposons at the donor site that are otherwise lost via TnpA-mediated excision, through formation of targeted DSBs and ensuing recombination (FIG. 5E).

### Example 5

### Methods for DNA and RNA Modification Using IStron-Derived Enzymes and Self-Splicing RNAs

Insertion sequences (IS) are the simplest mobile genetic elements found in bacteria which encode only the genes for their mobilization and retention. Those usually include two open reading frames, namely TnpA and TnpB. There are two main classes of IS elements, IS605 and IS607, which have homologous tnpB, but evolutionary unrelated tnpA genes. IS605 elements harbor a Y1 tyrosine transposase, which mediates transposition via single stranded DNA intermediate. IS607 TnpA is a serine resolvase, capable of cleaving and re-joining double stranded DNA.

Interestingly, some TnpA and TnpB homologs are encoded within group I introns, generating chimeric genetic elements called IStrons. These elements are not only mobile on the DNA level, due to TnpA and TnpB, but are phenotypically silent on the RNA level because the whole element is removed during splicing. IStrons can harbor TnpA and TnpB proteins related to either IS605 or IS607, suggesting multiple IS element acquisition events by group I intron during evolution. Some of the IStrons encoding proteins from IS607 elements were found in pathogenic bacteria species of Clostridium botulinum.

An IStron homolog from this species showed that TnpB (CboTnpB) is active for double-stranded DNA cleavage in E. coli. Here, TnpB from IS607 elements cleaves DNA when both TAM and target-complementary ωRNA guide are present and this activity is dependent on its RuvC active site. The same active site is also responsible for ωRNA maturation on the 5′ end. The transposase (CboTnpA) associated with this TnpB recognizes CboIStron ends and can excise the element from its native location. Lastly, the CboIStron can self-splice from the E. coli RNA transcript.

ωRNA covariation analyses. A ωRNA covariance model was built by performing blastp search for ChoTnpB protein (Table 3). Top 50 homologs were retrieved together with 3 kb sequence upstream and downstream of ChoTnpB gene. The sequences were clustered using CD-HIT and MAFFT was used to identify IStron boundaries. 220 nt upstream from 3′ end of mobile genetic element was extracted from each member and a structure-based multiple alignment was then performed using mLocARNA with the following parameters:
--max-diff-am 25--max-diff 60--min-prob 0.01--indel-50--indel-open-750--plfold-span 100--alifold-consensus-dp

The resulting alignment with structural information was used to generate a new ωRNA covariance model with the Infernal suite, refined with Expectation-Maximization from CMfinder and verified with R-scape at an E-value threshold of 1e-5. The resulting ωRNA covariance model was used with cmsearch to discover new ωRNAs within top 1000 hits from blastp search for CboTnpB protein. The new identified sequences were used to iterate the initial model.

Cloning Expression vectors (pEffector) and target plasmids (pTarget), were designed as described previously using a variety of methods, including inverse (around-the-horn) PCR, Gibson assembly, restriction digestion-ligation, and ligation of hybridized oligonucleotides. pEffector encodes a codon optimized CboTnpB (or CboTnpB (D190A)) and an ωRNA under the control of two separate constitutive promoters on a pCDF-Duct-1 vector. In this experiment, target plasmids (pTarget) were designed to encode a 40-bp complementary target sequence to ωRNA guide, on a pCOLA backbone. Representative plasmid sequences are listed in Tables 7 and 8.

Targeted plasmid DNA cleavage in E. coli Plasmid interference assays were performed in E. coli str. K-12 substr. MG1655. The cells were transformed with pEffector plasmids and single colony isolates were selected to prepare chemically competent cells. These cells were transformed with 200 ng of pTarget plasmids by heat shocking at 42° C. for 30 sec, followed by recovery at 37° C. for 1 h. The cells were then spun down at 4000 g for 5 min and resuspended in 30 μl of MilliQ H₂O. Cells were then serially diluted (10×) and plated on LB-agar media with spectinomycin (100 μg ml⁻¹) and kanamycin (50 μg ml⁻¹). Cells were grown for 24 h at 37° C. and plates were imaged in an Amersham Imager 600.

TAM library assays and NGS library prep. To unbiasedly determine ChoTnpB TAM sequence a plasmid library with 6 degenerate nucleotides 5′ of the target sequence was used. DNA solutions containing 500 ng of the TAM plasmid library (pSL4841) and 500 ng of plasmids encoding either ChoTnpB with a targeting ωRNA (pSL5002) or ChoTnpB with a non-targeting ωRNA (pSL4902) were co-transformed in electrocompetent E. coli BL21 (DE3) cells according to the manufacturer's protocol (Sigma-Aldrich). Cells were serially diluted on large bioassay plates containing LB agar supplemented with spectinomycin (100 μg ml⁻¹) and kanamycin (50 μg ml⁻¹). Approximately 400,000 CFUs were scraped from plates, representing 100× coverage of each library member. Plasmid DNA was isolated using the Qiagen CompactPrep Midi Kit. Illumina amplicon library for NGS was prepared through a 2-step PCR amplification. In brief, ˜25 ng of plasmid DNA recovered from TAM assay was used in each 1^{st} step PCR amplification reaction with primers flanking the degenerate TAM library sequence and containing universal Illumina adaptors as 5′ overhangs. Amplification was carried out using high-Fidelity Q5 DNA Polymerase (NEB) for 15 thermal cycles. Samples from 1^{st} step PCR amplification were diluted 20-fold and amplified for 2nd step PCR in 10 thermal cycles with primers containing indexed p5/p7 sequences. Reactions were verified by analytical gel electrophoresis. Sequencing was performed with a single-end run using a MiniSeq High Output Kit for 75-cycles (Illumina).

Analyses of NGS TAM library data. Analysis of TAM depletion library was performed using a custom Python script. Demultiplexed reads were filtered to remove reads that did not contain a perfect match to the 58-bp sequence upstream of the degenerate sequence for any i5-reads. For reads that passed this filtering step, the 6-nt degenerate sequence was extracted and counted. The relative abundance of each degenerate sequence in a sample was determined by dividing the degenerate sequence count by the total number of sequence counts for that sample. Then, the fold-change between the output and input libraries was calculated by dividing the relative abundance of each degenerate sequence in the output library by its relative abundance in the input library, and then log 2-transformed. Sequence logos were constructed by taking the 50 most depleted sequences and generated using WebLogo (v2.8).

RIP-seq to capture mature ωRNA. RNA-immunoprecipitation (RIP) followed by sequencing was used to detect mature ωRNA bound by ChoTnpB. Cells expressing 3×FLAG-CboTnpB and bioinformatically predicted ωRNA were grown until they reached an exponential phase, then pelleted, resuspended in lysis buffer and sonicated. Resulting lysate was centrifuged and supernatant left to incubate overnight at 4° C. with Dynabeads, conjugated with anti-FLAG antibodies. The bound fraction was eluted, with TRIzol and chloroform, followed by RNA purification using Zymo RNA Clean & Concentrator Kit. Purified RNA was fragmented and treated with Turbo DNase, purified using Zymo RNA Clean & Concentrator Kit and used for library preparation with NEBNext Small RNA Library Prep Set for Illumina. Sequencing was performed with a paired-end run using a MiniSeq High Output Kit for 150-cycles (Illumina). Resulting reads were mapped using BWA and custom Python script.

Excision assay with CboTnpA. To monitor excision MG1655 cells were transformed with TnpA expressing plasmid. The obtained transformants were used to make chemically competent cells that were then transformed with donor DNA containing plasmid. Double-transformants were plated on LB agar with selective antibiotics and IPTG for the induction of TnpA expression. Resulting colonies were scraped from the plate and lysed by boiling at 95° C. for 10 min. The lysate was centrifuged, and the supernatant was used for PCR.

Splicing assay of CboIStron. Cells expressing CboIStron were grown until they reached exponential phase. They were pelleted and total RNA was extracted using TRIzol and chloroform. RNA was concentrated using NEB Monarch RNA Cleanup Kit. 200 ng of total RNA were treated with dsDNAse and reverse-transcribed using SuperScript IV Reverse Transcriptase. Resulting cDNA was used for PCR.

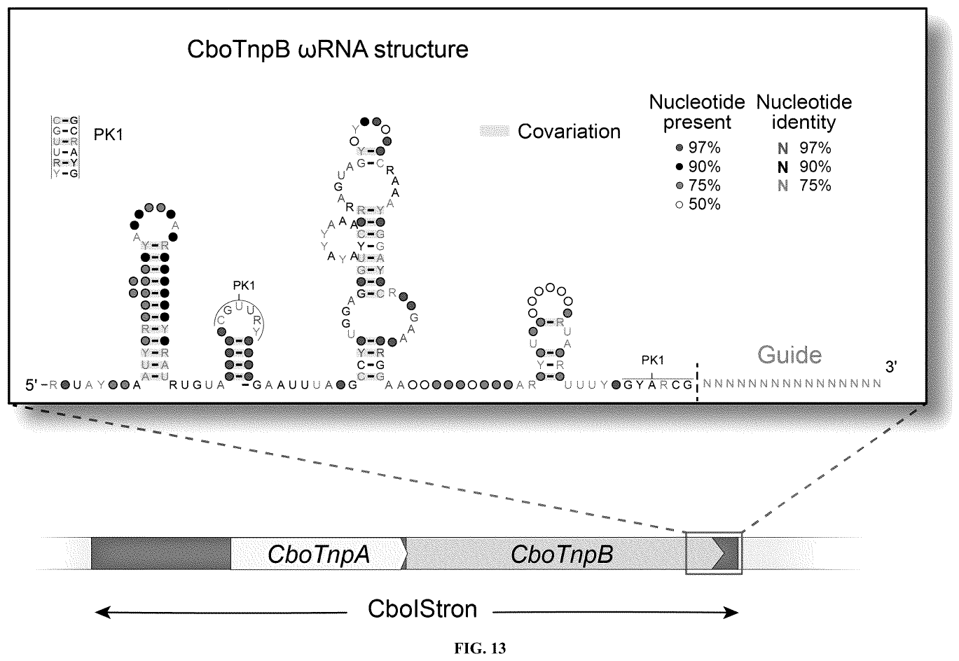

Investigating ChoTnpB DNA cleavage It was hypothesized that ChoTnpB could be binding an ωRNA, which was likely encoded at the 3′ end of mobile genetic element. From the knowledge about TnpB from IS605 elements, the RNA was expected to be ˜200-250 nt in length and could be partially overlapping with TnpB coding sequence. ChoTnpB homologs were aligned and a covariation model for the expected coding region of ωRNA was built. The 3′ end of IStron has a conserved secondary structure indicating that the transcript might be functional at RNA level (FIG. 13). Therefore, 220 nt upstream of 3′ IStron end were used as a scaffold for the guide RNA. A likely ChoTnpB target could its donor joint, which is formed at the genomic location once IStron is excised by TnpA. In the case of Clostridium botulinum IStron (CboIStron), the motif upstream of mobile genetic element is 5′-TGG, which were selected to be used as TAM. Downstream of it a native sequence found 3′ of IStron was cloned in and ωRNA guide were designed to be complementary to it.

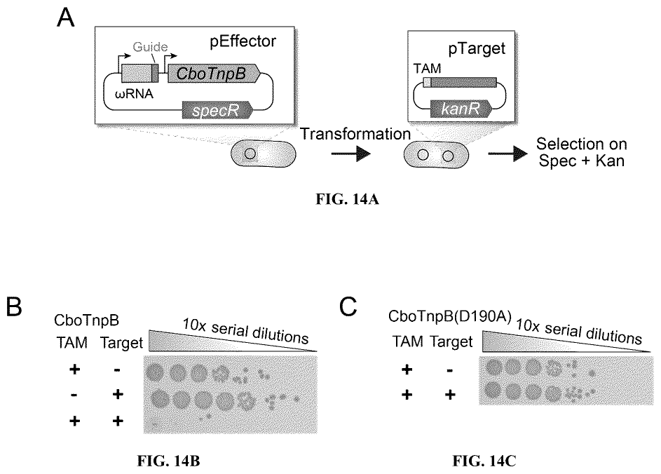

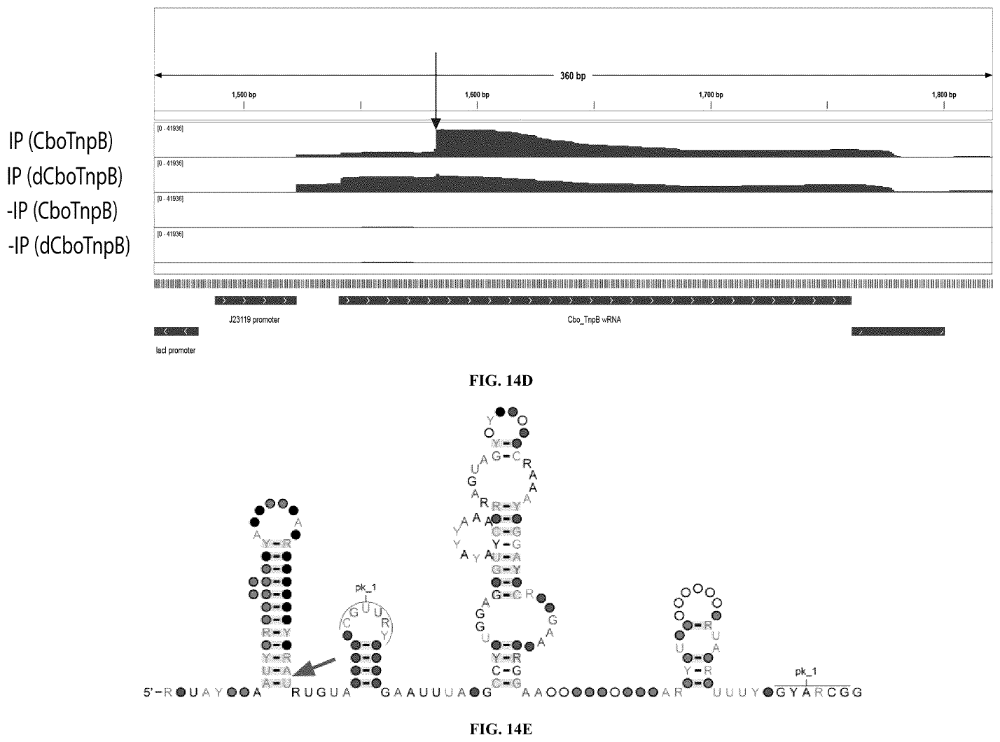

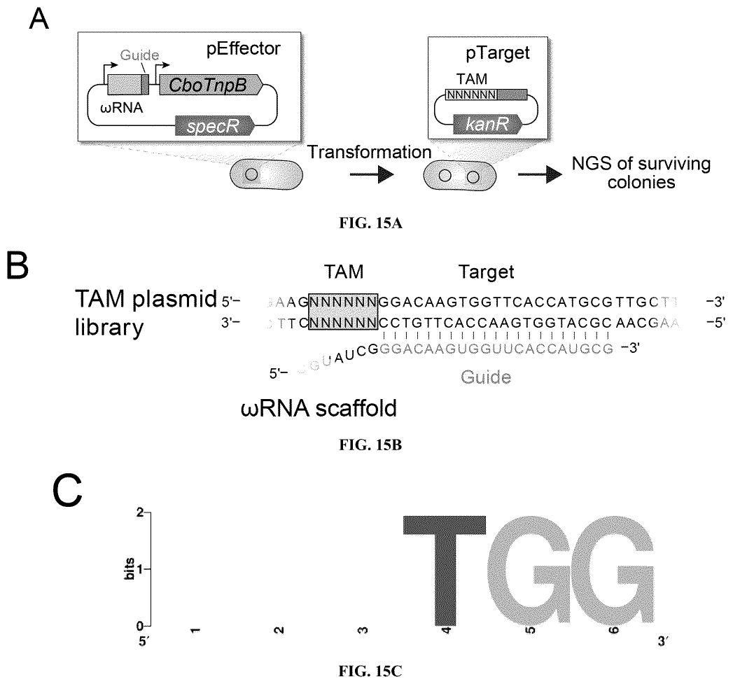

To test whether ChoTnpB is able to cleave double-stranded DNA in E. coli a plasmid interference assay was designed (FIG. 14A). E. coli was transformed with a pEffector plasmid (encoding CboTnpB and ωRNA), and then transformed with pTarget plasmids. When the target is recognized and cleaved, bacteria lose resistance to antibiotic encoded by pTarget. ChoTnpB DNA cleavage utilized both TAM and ωRNA guide complementarity to the target sequence (FIG. 14B).

TnpB proteins have a predicted RuvC nuclease domain, which is also found in widely studied class II CRISPR-Cas nucleases (Cas9 and Cas 12). By mutating one of its active site residues ChoTnpB loses its activity confirming that DNA cleavage is RuvC-dependent (FIG. 14C).

Additionally, RNA sequencing was performed to determine the mature form of CboTnpB ωRNA. Using RNA immunoprecipitation followed by sequencing (RIP-seq) a 197 bp long RNA which precipitated together with ChoTnpB was detected. Interestingly, a sharp processing site at 5′ end was observed only when ChoTnpB RuvC domain was intact, suggesting its role in ωRNA maturation (FIG. 14D). There was no significant difference between 3′ end boundary between nuclease active and dead ChoTnpB variants, suggesting that it is being truncated by cellular nucleases. When looking at the covariation model, ChoTnpB cleavage spot lands right at the base of a highly conserved stem loop, suggesting that it might be important for maturation (FIG. 14E).

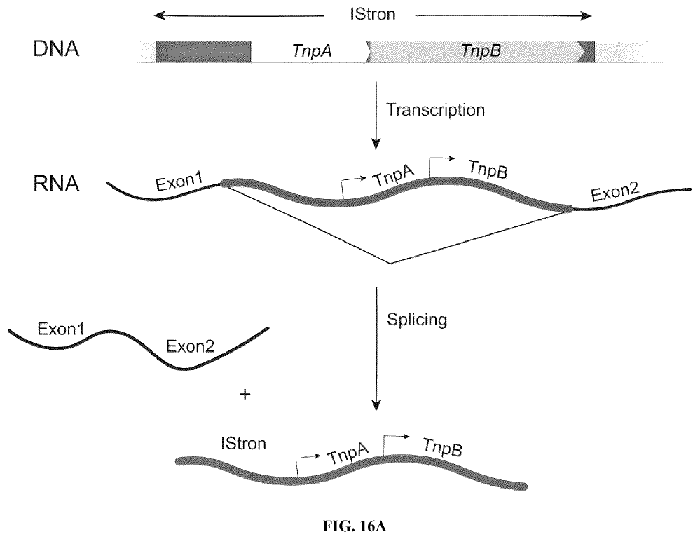

Reprogramming of ChoTnpB To unbiasedly probe how stringent TAM preference CboTnpB has, a library cleavage experiment was performed (FIG. 15A). A similar experimental setup was used, but instead of a single pTarget a plasmid library that has a degenerate 6N nucleotide sequence was used. The ωRNA guide sequence was also changed to be complimentary to the sequence downstream of the 6N motif (FIG. 15B). By plating on selective media and harvesting the surviving clones, the most depleted library members were identified by NGS. This experiment confirmed that ChoTnpB can be reprogrammed to cleave a different DNA target than the native one, and 5′-TGG is the most favored TAM sequence, which is readily recognized and cleaved by ChoTnpB (FIG. 15C).

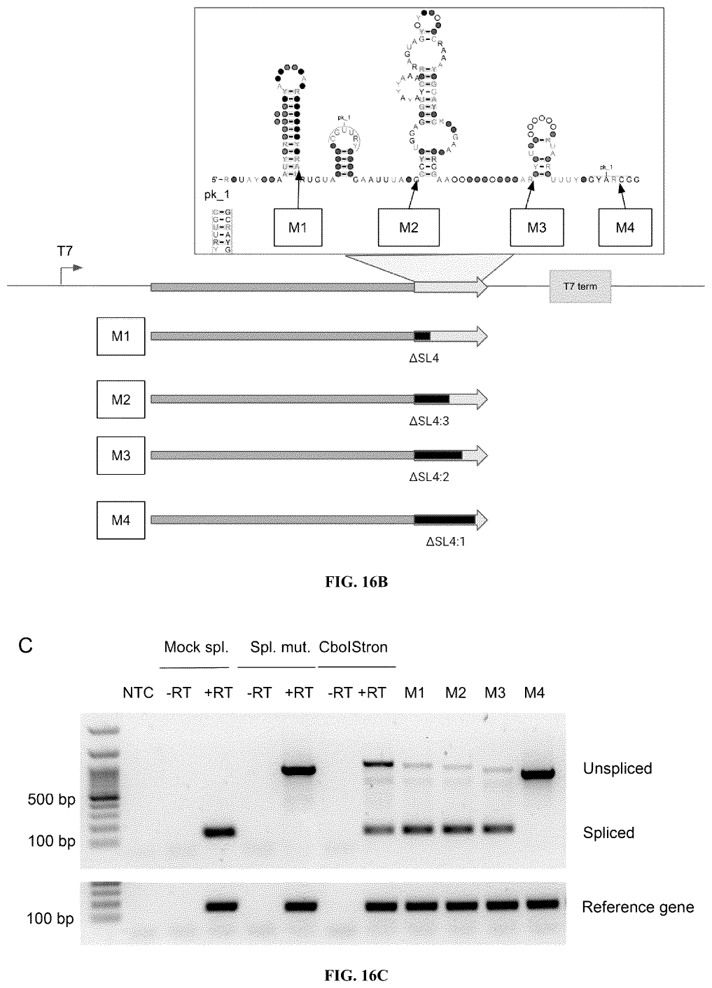

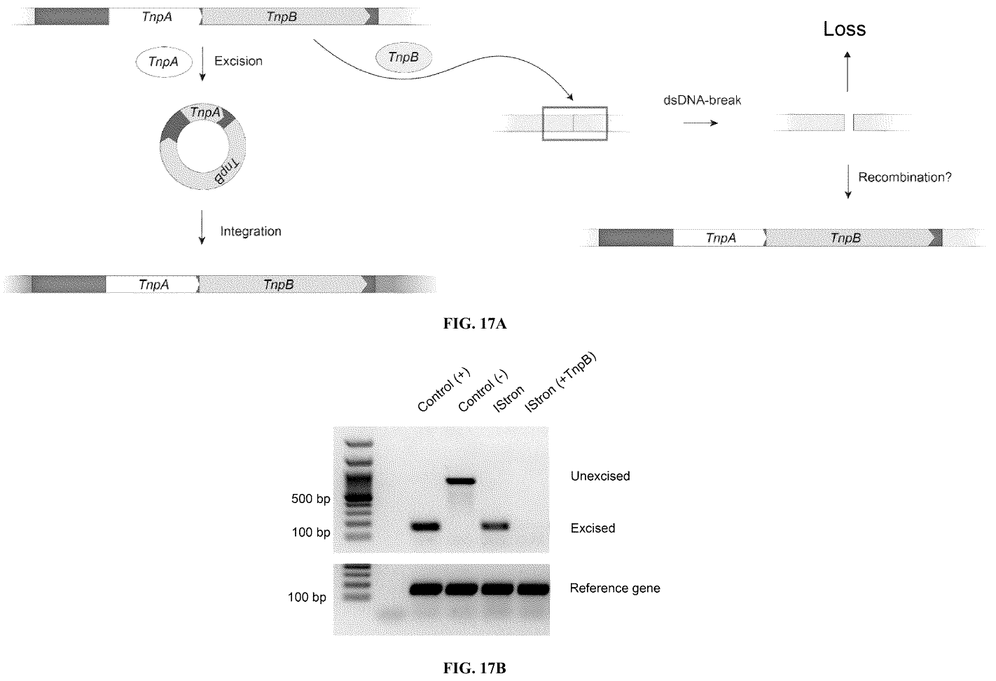

CboIStron exhibits self-splicing in E. coli Due to their left and right end similarity to group I introns, IStrons are predicted to be silent mobile genetic elements, capable of cleaving themselves out of RNA transcript (FIG. 16A). To investigate if CboIStron retains self-splicing ability a minimal IStron (lacking tnpA and tnpB genes) was constructed and RT-PCR was performed to capture splicing products. Being guided by predicted IStron right end (ωRNA) fold some predicted structural features were removed to test minimal requirements for intron splicing (FIG. 16B). This assay revealed that removing up to three inner stem loops leads to increased splicing activity, but splicing was ablated when the outer-most stem loop is lost (FIG. 16C).

CboTnpA can excise IStron from its genomic location Just as the self-splicing activity can remove IStron from RNA transcript, so can CboTnpA permanently excise the element form any gene at the DNA level and integrate it elsewhere (FIG. 17A). The activity of CboTnpA was reconstituted in E. coli and monitored excision by doing PCR to amplify the excision junction. CboTnpA effectively excised minimal IStron, but the effect was lost when IStron encoded CboTnpB (FIG. 17B).

### Example 6

### Methods for Programmable RNA-Guided DNA Cleavage Using TnpB Homologs

Recent genome editing methods have largely depended on Cas9- or Cas 12-like nucleases with an associated guide RNA (sometimes referred to as gRNA or sgRNA). These nucleases are guided to a target site complementary to their respective sgRNA and generate DNA double strand breaks (DSBs) at the target site. In some embodiments, a ssDNA or dsDNA donor template may be introduced as well for homologous recombination to occur, leading to the knock-in of a desired DNA sequence. In some embodiments, one active site of the nuclease may be inactivated via specific amino acid mutations, resulting in a “nickase”. In some embodiments, the nuclease protein may be catalytically inactive. In some embodiments, the nuclease can be fused to various effector proteins, including, but not limited to, a reverse transcriptase, a DNA deaminase, a transcriptional activator, or a transcriptional repressor. However, current editing methods are still limited due to the large coding size of typical genome editors, and many have focused on identifying smaller Cas9 orthologs to enable more efficient delivery methods. Furthermore, Cas9 orthologs from thermophilic species lend further opportunities for improved genome editing, as thermostable systems show improved behavior in human cells. TnpB and IscB proteins derived from G. stearothermophilus are appealing nucleases for genome editing given their small reading frame and potential thermostability.

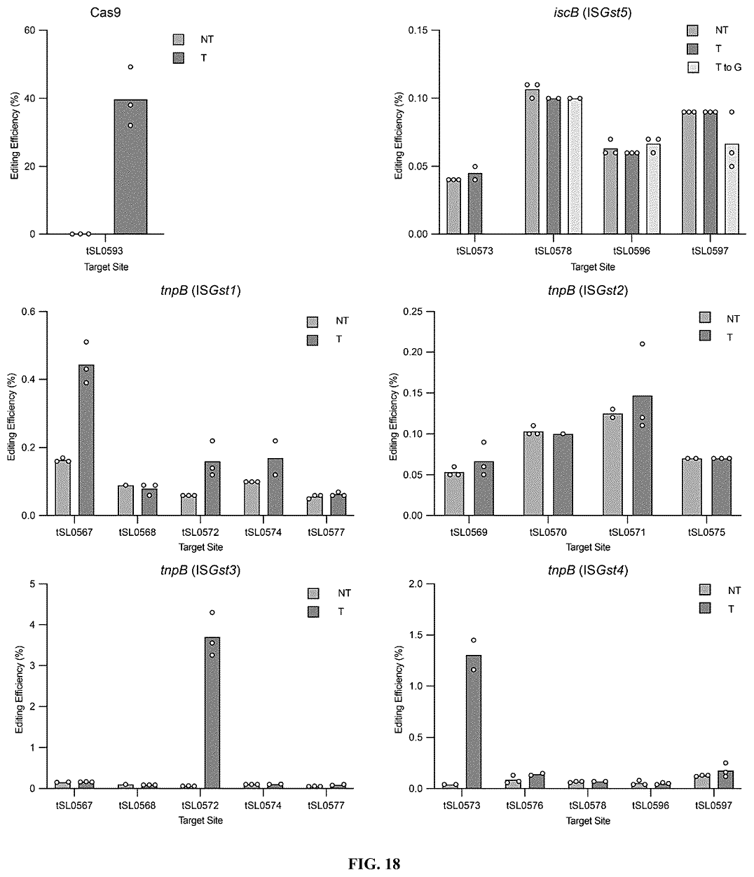

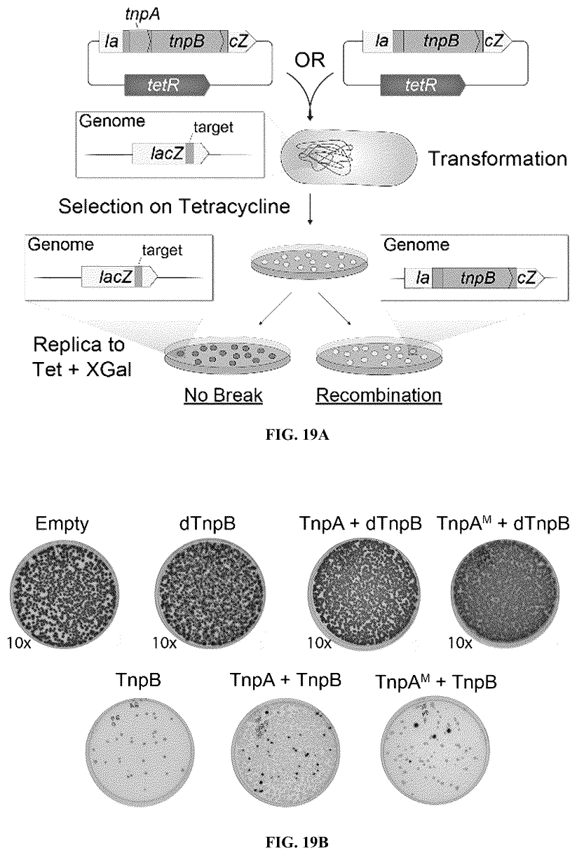

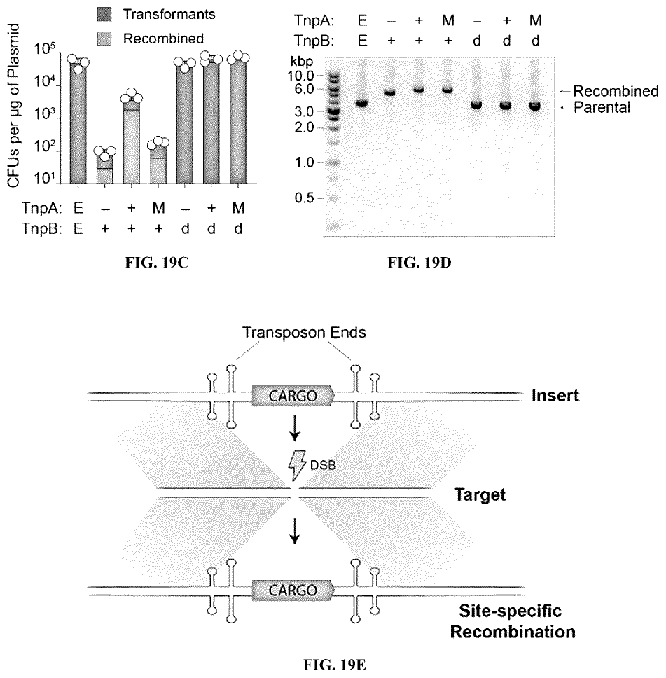

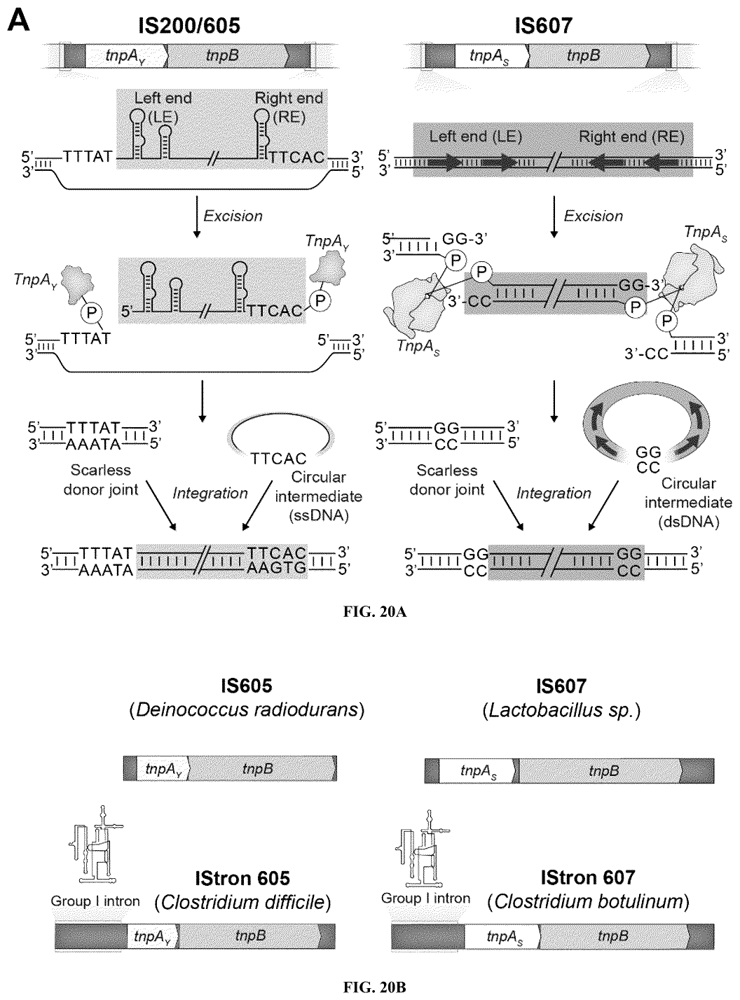

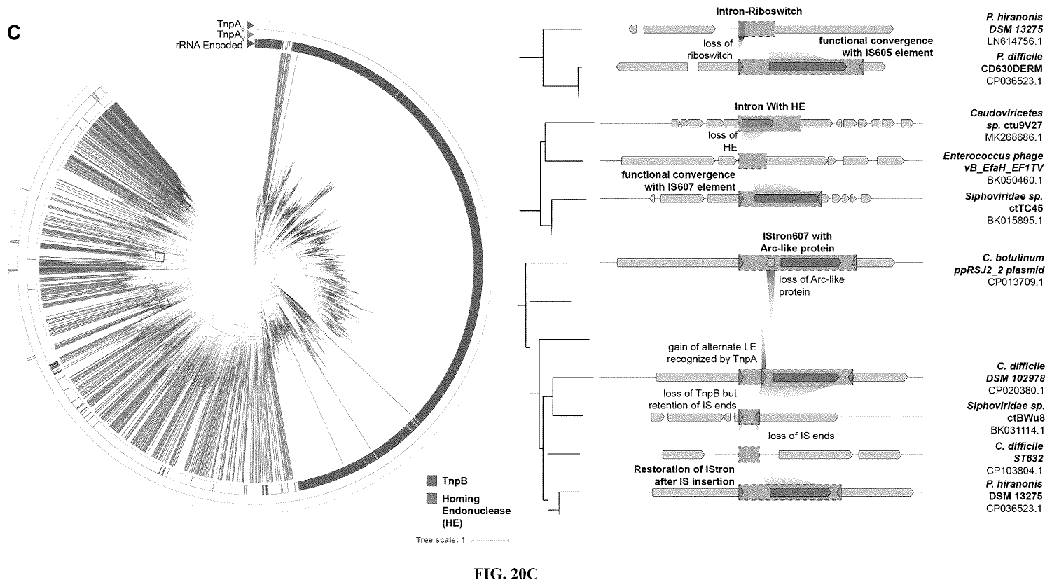

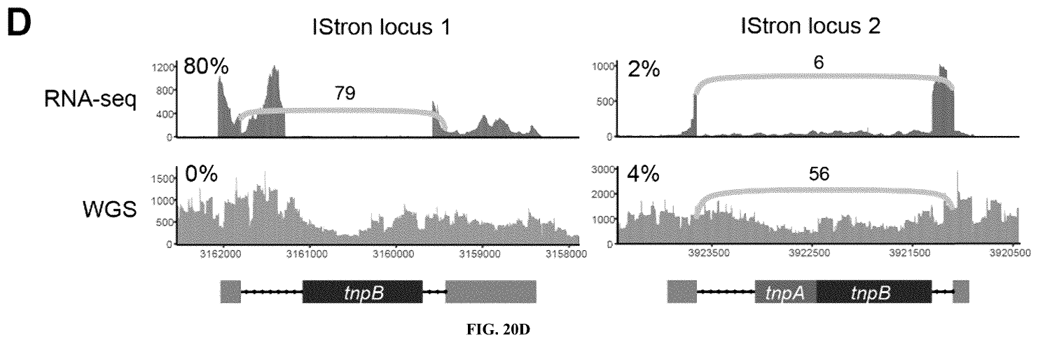

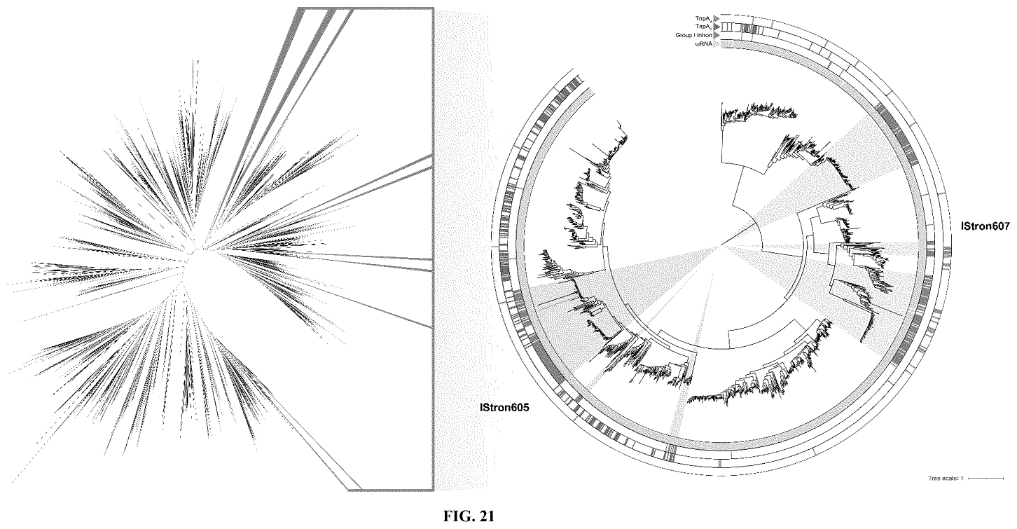

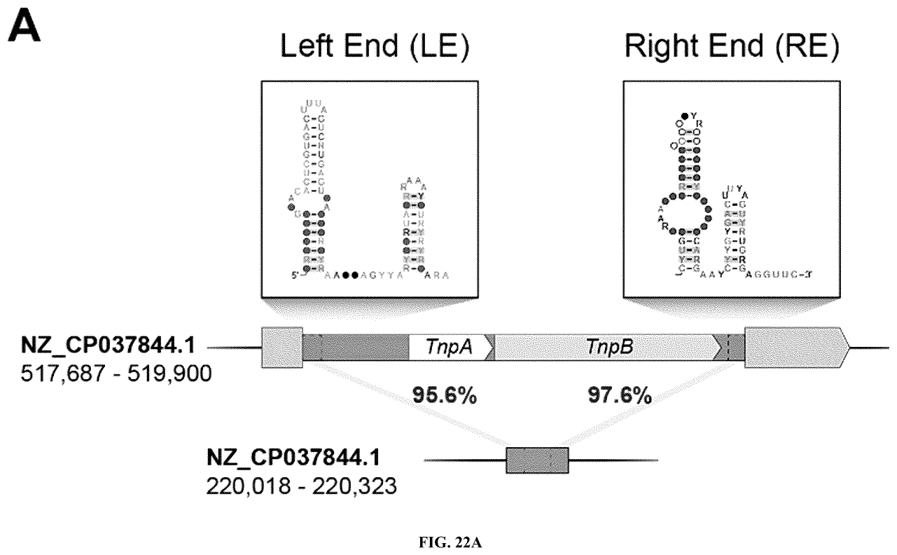

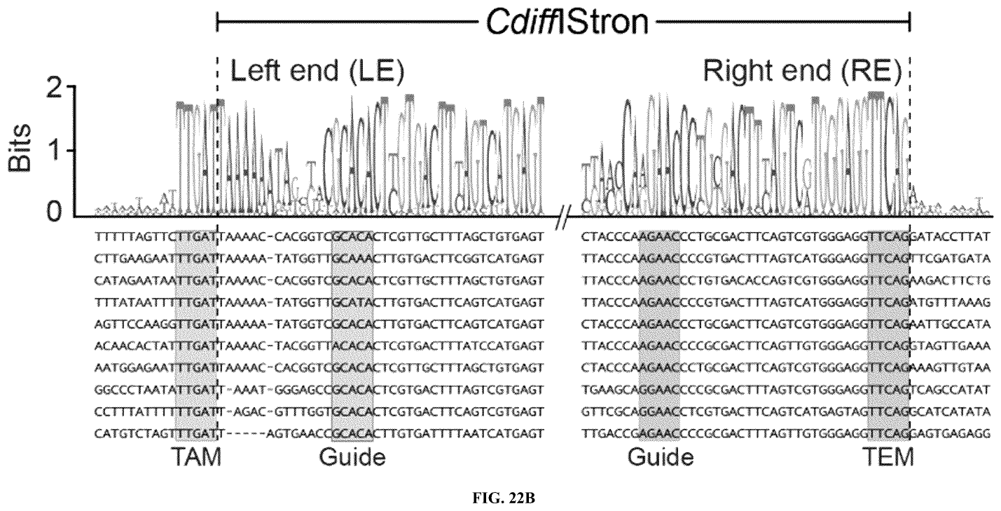

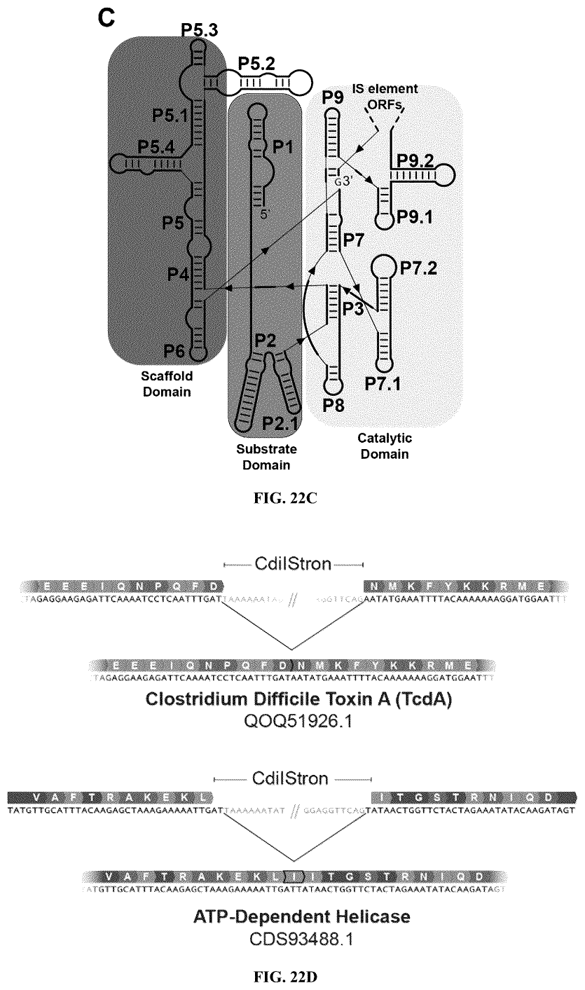

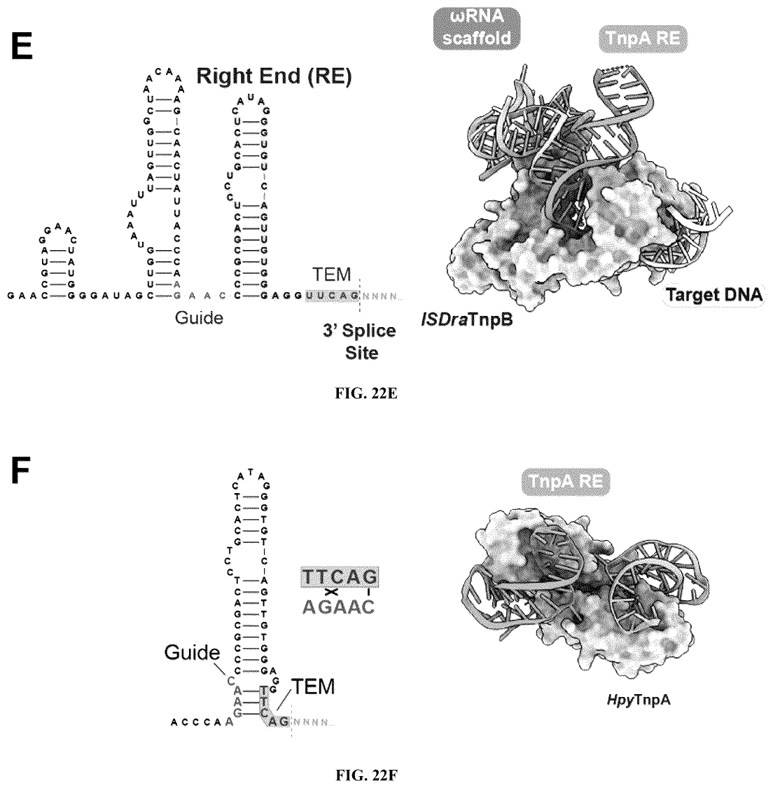

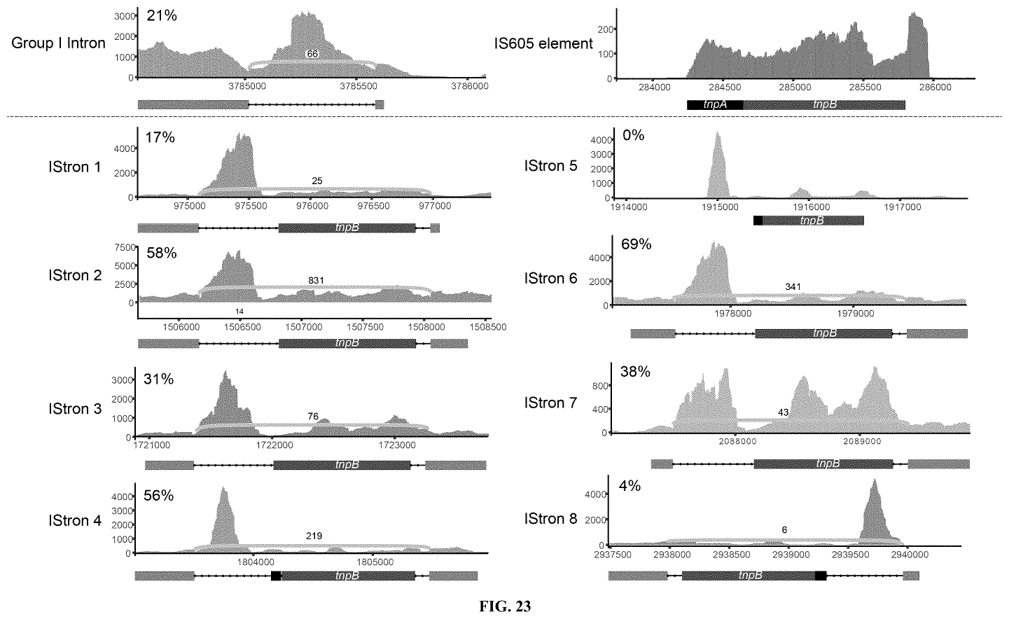

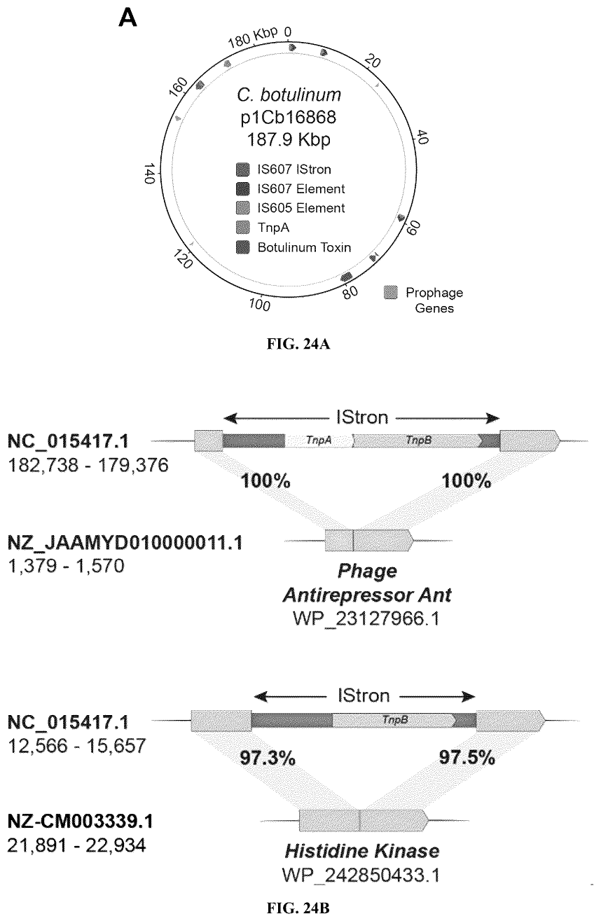

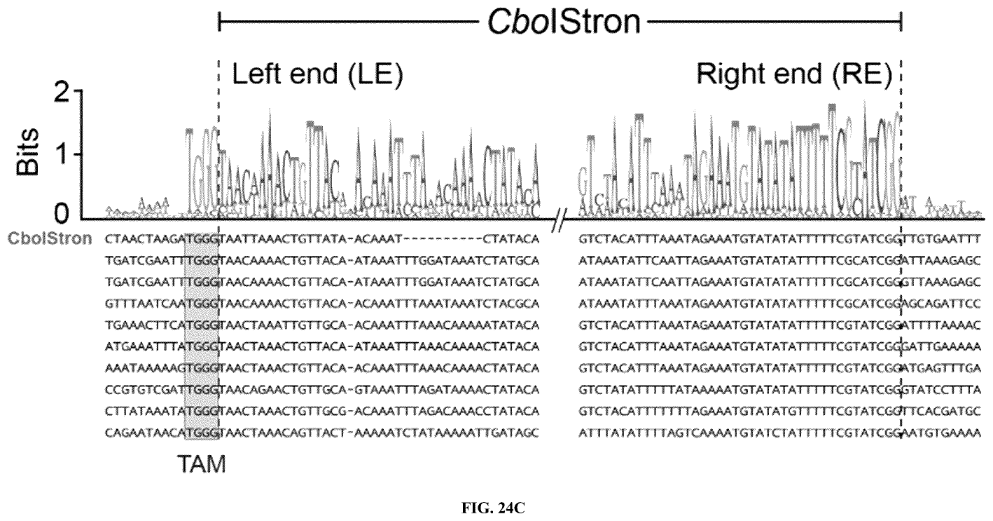

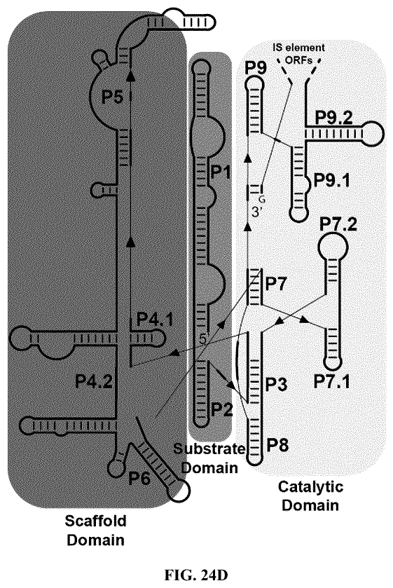

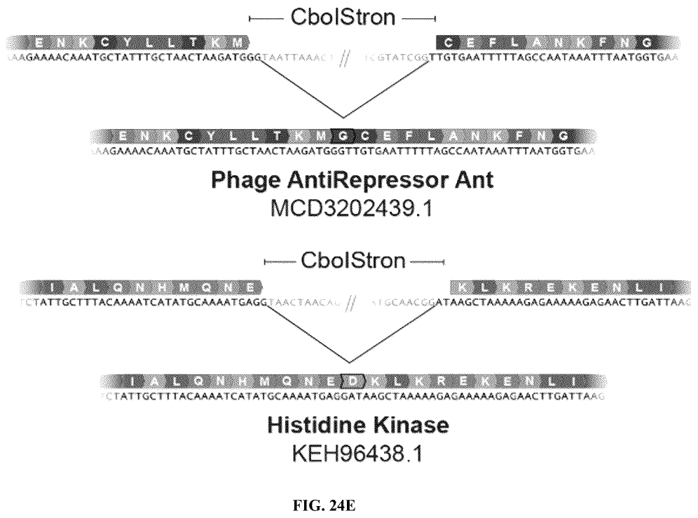

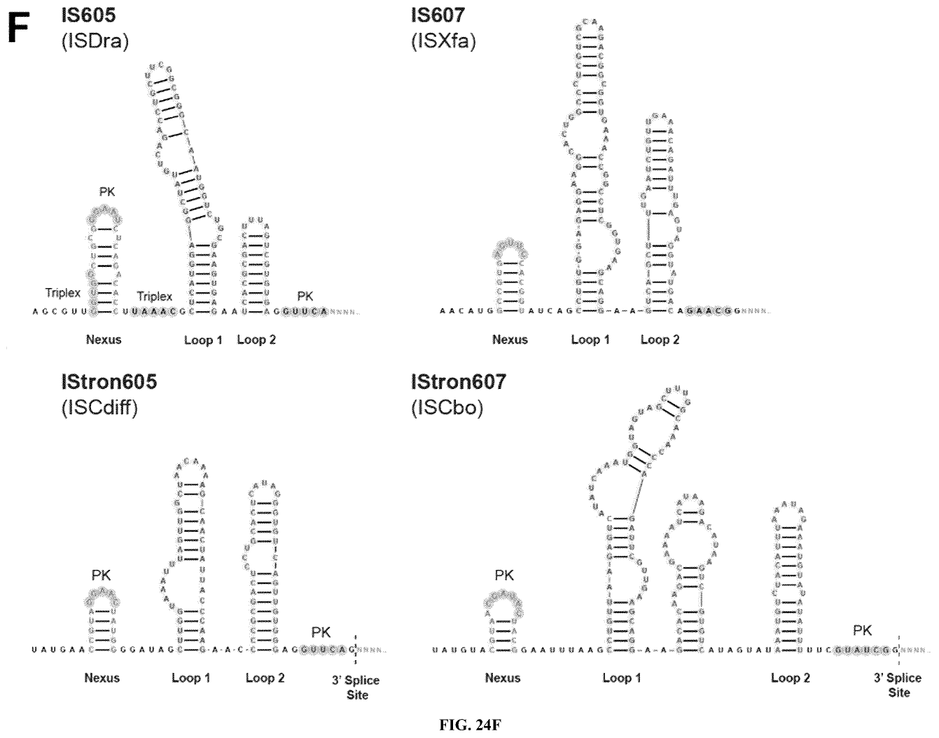

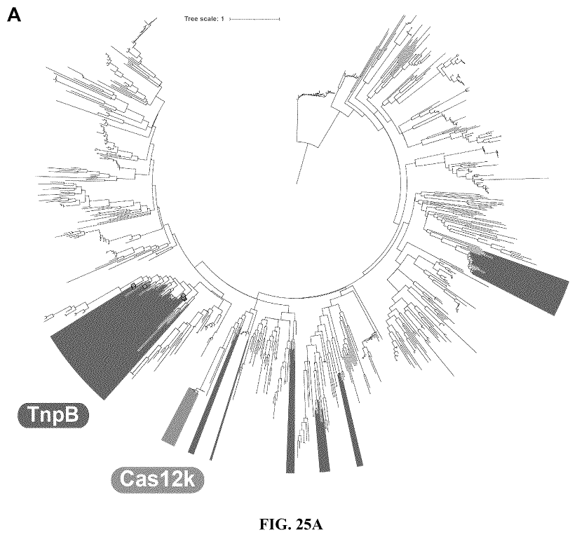

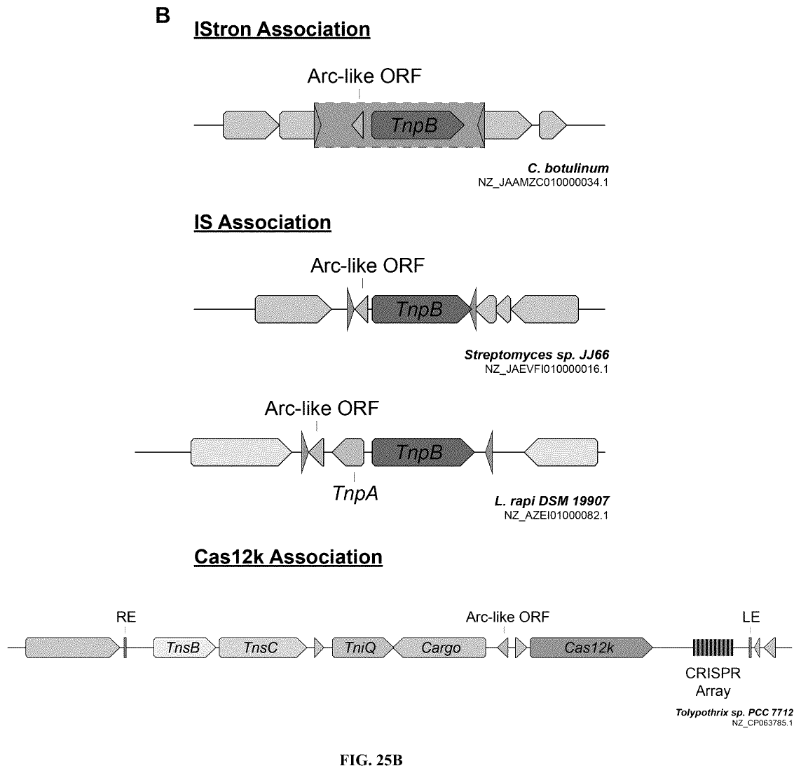

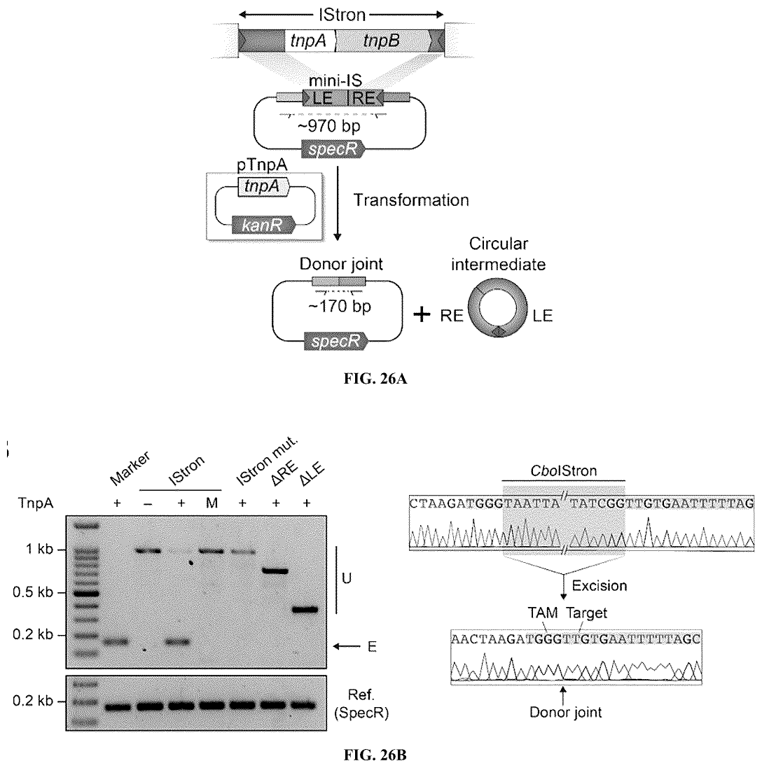

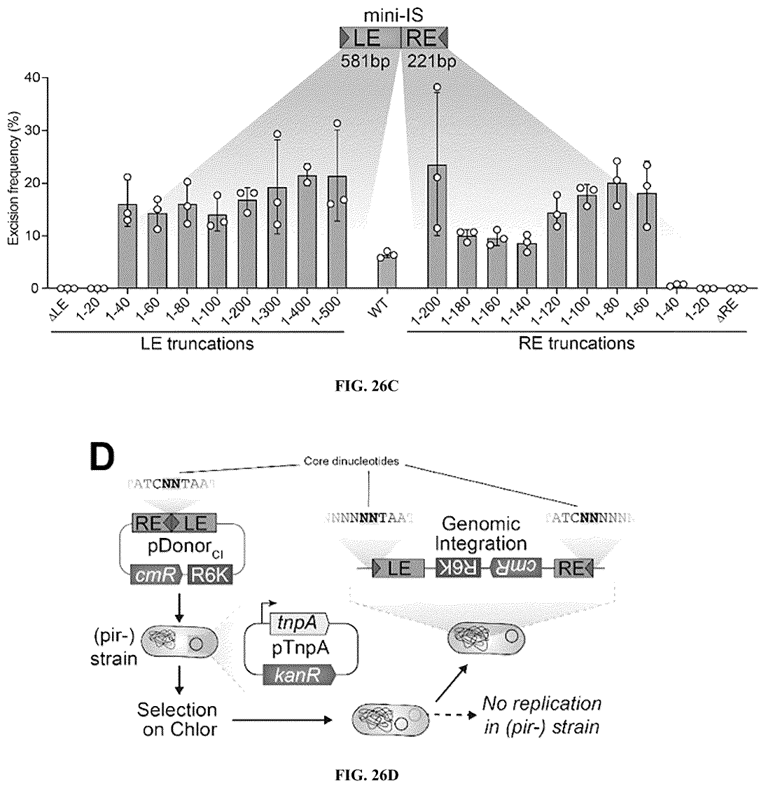

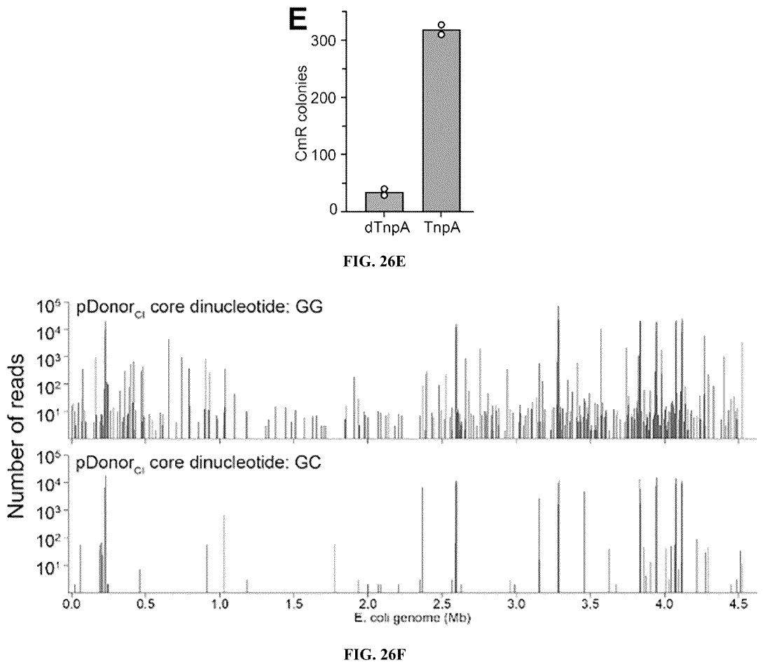

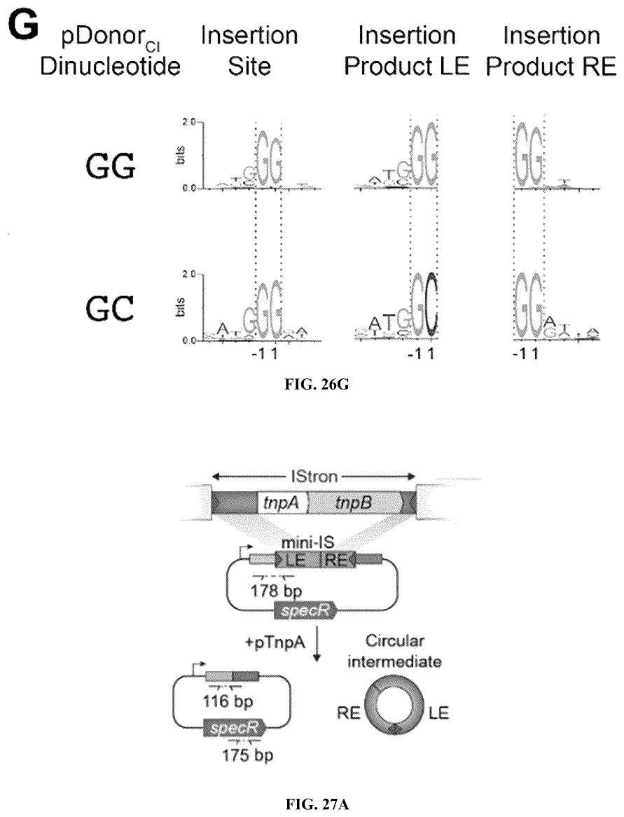

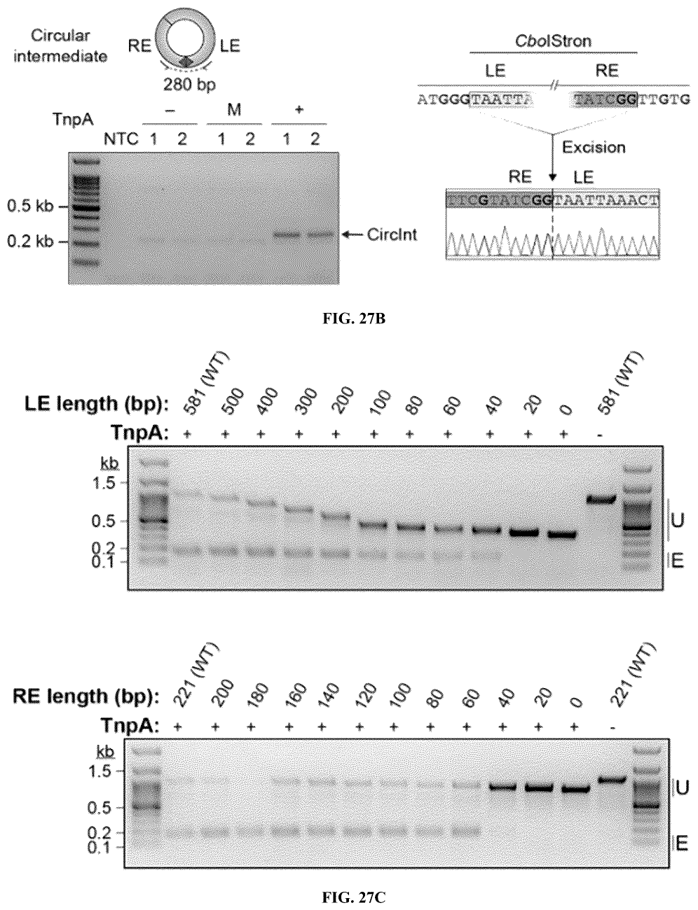

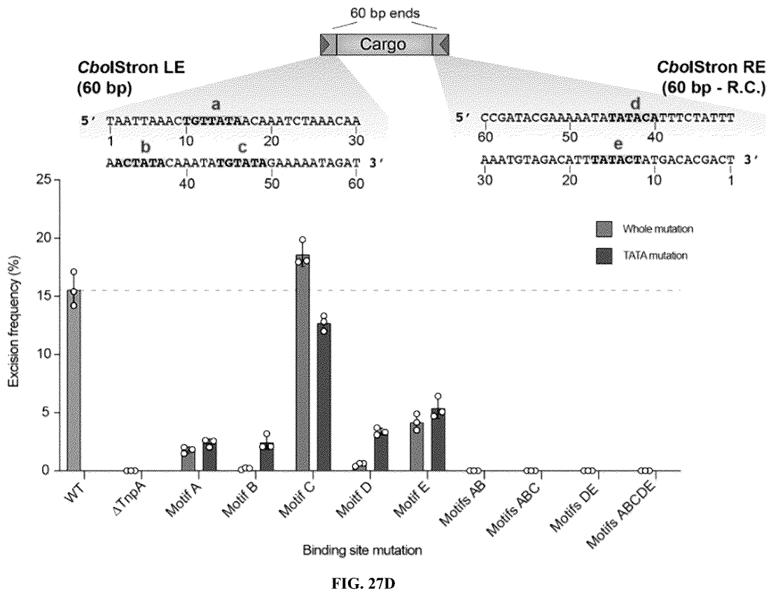

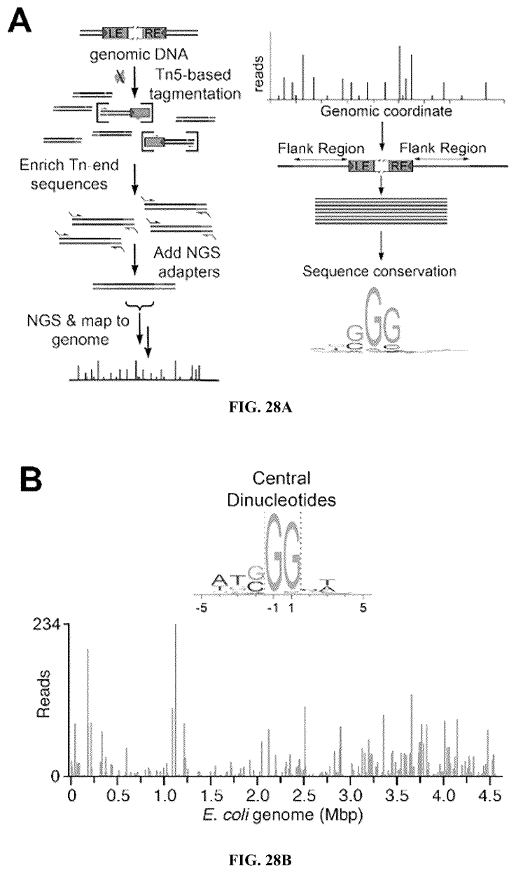

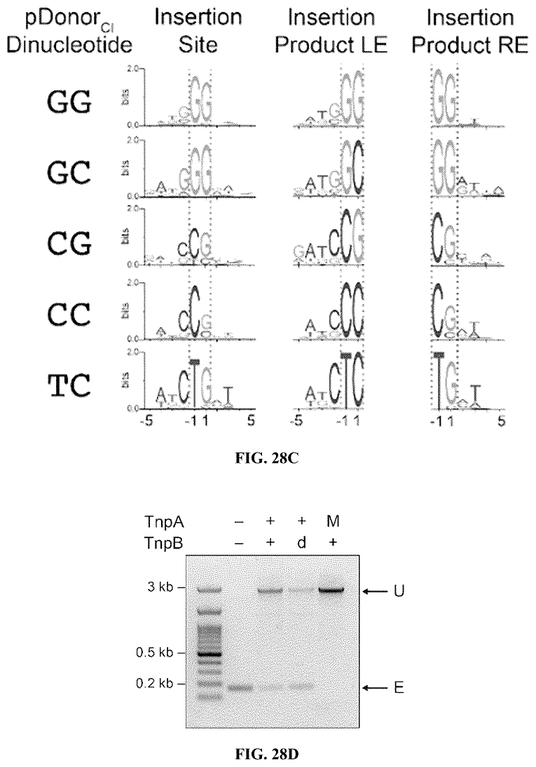

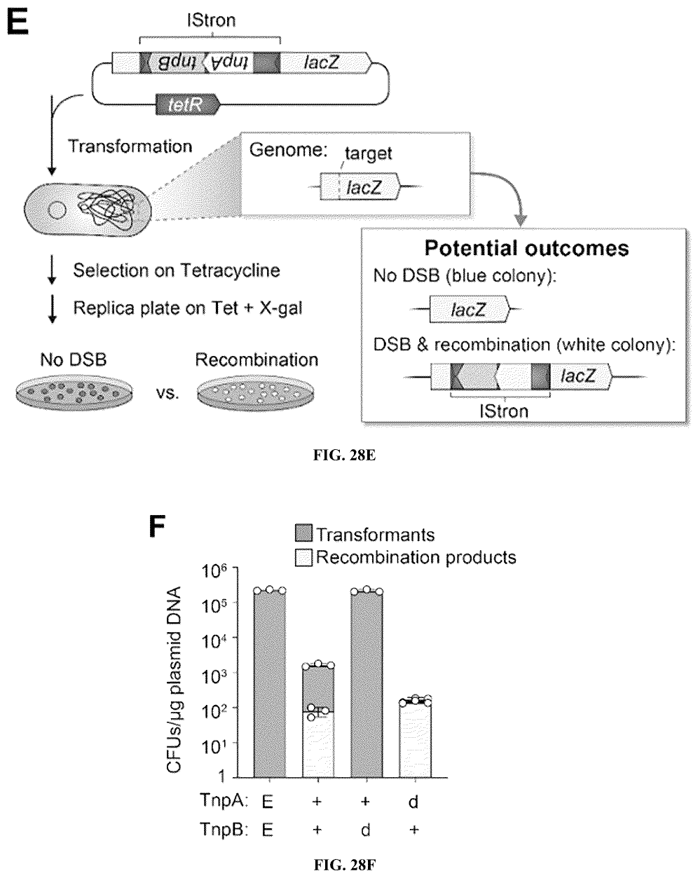

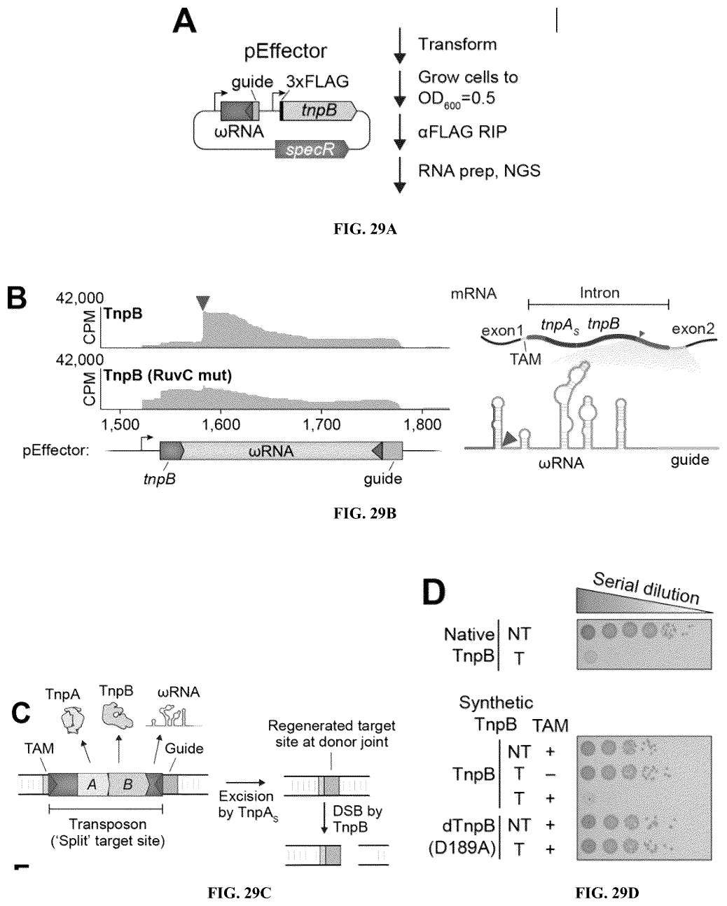

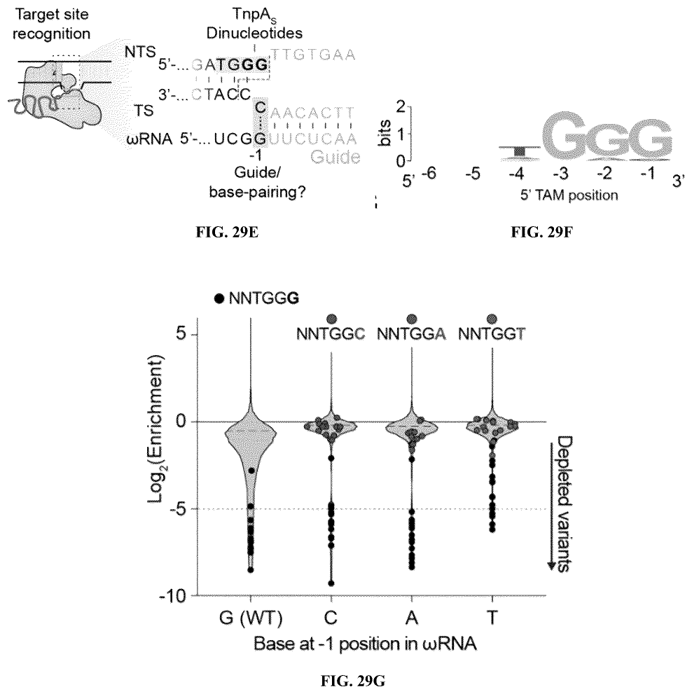

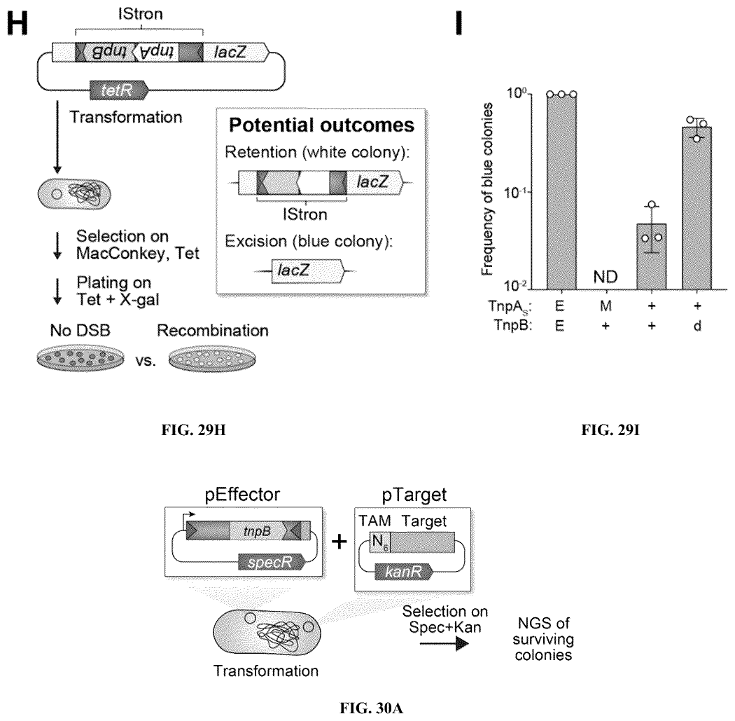

Investigating TnpB and IscB editing efficiencies in human cells To investigate the editing efficiencies of TnpB and IscB proteins, all components were human-codon optimized and were appended with a C-terminal bipartite-NLS sequence. Omega RNA sequences (referred to as ωRNA) were cloned immediately downstream of a U6 promoter, with a poly-T terminator immediately downstream. Various target sites within the HEK3 locus were targeted for each system, with the appropriate TAM sequences chosen, where TAM represents the transposon/target-adjacent sequence (Table 7). Cells were seeded in 48-well plates approximately 18-24 hours prior to transfection, and cells were transfected with 200 ng of a TnpB or IscB expression plasmid, 200 ng of the ωRNA expression plasmid, and 10 ng of a drug marker to select for transfected cells. Cas9 and a gRNA expression plasmid targeting HEK3 was used as a positive control comparison. Transfected cells were selected for the transfection marker for 3 days, and then harvested for editing analysis. Samples were analyzed via targeted PCR, next generation sequencing, and CRISPResso2 analysis. TnpB and IscB proteins exhibited a range of detectible editing efficiencies across target sites at the HEK3 locus. Editing efficiencies are reported in FIG. 18. Several systems, including TnpB (derived from ISGst3) and TnpB (derived from ISGst4) exhibited robust editing efficiencies with single-digit efficiencies.

### Example 7

### Methods for Increasing Site-Specific DNA Recombination Efficiencies Using TnpA (Y1) Transposase

TnpB Insertion sequences (IS) are compact and pervasive transposable elements found in bacteria and archaea, which canonically encode only the genes for their mobilization and maintenance. IS200/IS605 transposons undergo ‘peel-and-paste’ transposition catalyzed by a TnpA transposase, but intriguingly, they also encode diverse, TnpB- and IscB-family proteins that are evolutionarily related to the CRISPR-associated effectors Cas12 and Cas9, respectively. TnpB-family enzymes function as RNA-guided DNA endonucleases, but the broader biological role and their associated activity with TnpA has remained enigmatic. Co-expression of TnpA and TnpB to direct targeted double-stranded breaks (DSBs) results in a substantial increase in recombination frequencies, surpassing rates observed with TnpB alone. The hyper-recombination frequency mediated by the TnpA transposase can be used to increase DSB-dependent site-specific recombination, overcoming limitations in the low efficiency of DSB-dependent recombination for site-specific DNA integration.

TnpA (Y1) increases site-specific DNA recombination following DSBs. IS200/IS605 elements encode two genes: tnpA, which encodes a transposase containing a catalytic tyrosine residue responsible for DNA excision and integration of the mobile genetic element; and tnpB or iscB, which encode RNA-guided DNA nucleases termed TnpB or IscB. While the function of each gene in separation has been determined, the role of these proteins in combination has been unknown.

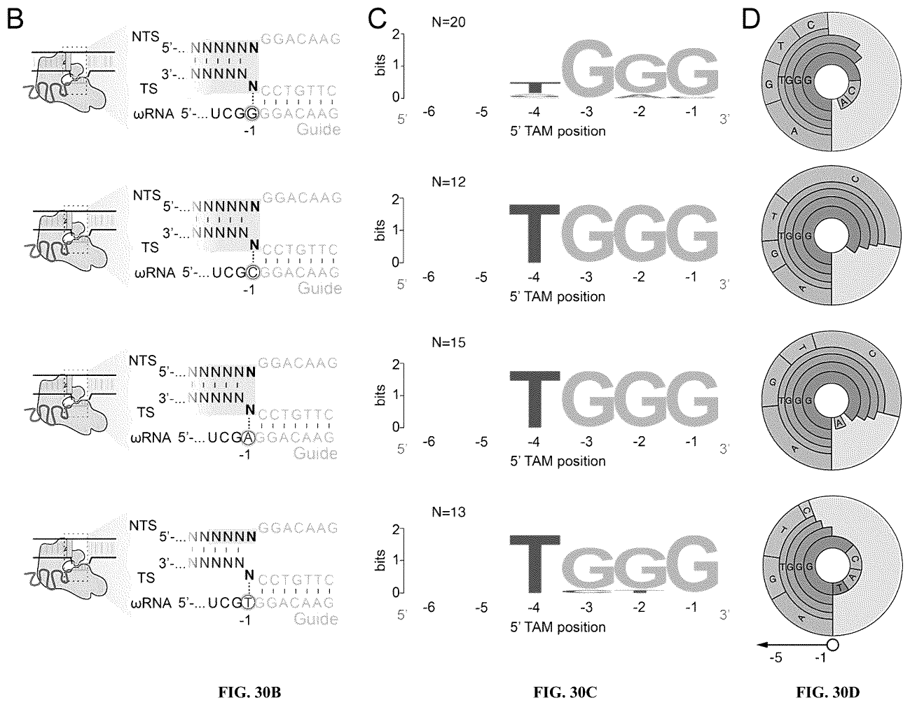

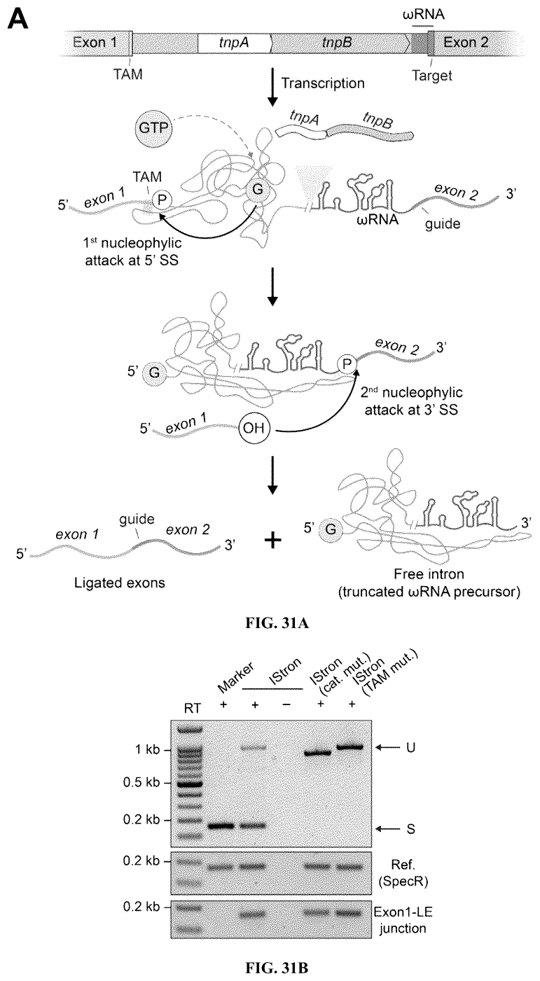

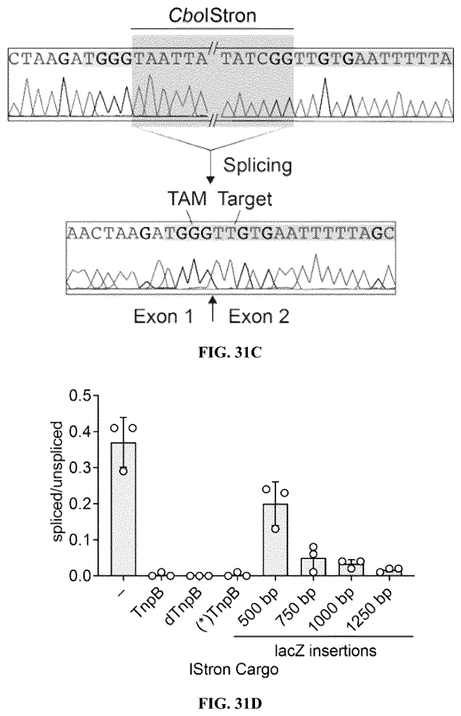

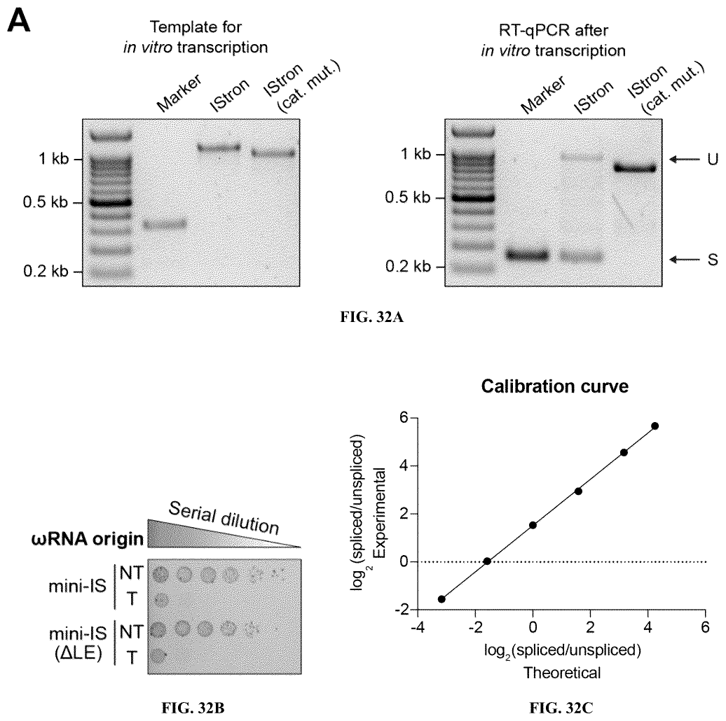

IS200/IS605-like elements couple TnpA-mediated excision, resulting in a scarless excision event, with TnpB RNA-guided DNA cleavage, that targets the excised element during transposition, leading to DNA recombination and reinstallation of the IS element back into the donor site. An assay was developed to monitor recombination events occurring between a plasmid-encoded IS element inserted into full-length lacZ, and its corresponding lacZ donor joint site encoded on the genome (FIG. 30A). Upon E. coli transformation, TnpB and ωRNA expression leads to targeted DNA double-strand breaks within the genomic lacZ locus, leading to one of two potential outcomes: cell death from unresolved DNA damage, or cell survival via homologous recombination with the lacZ locus on the ectopic plasmid, effectively copying the ISGst2 element into the genome and disrupting the target site. These outcomes were scored by quantifying the number of surviving colonies that were lacZ⁺ (uncleaved/mutated, blue colony phenotype) or lacZ⁻ (recombination products, white colony phenotype; FIG. 30A).

An approximate 500× reduction in cell survival was observed after transformation with the WT ISGst2 element, and this effect was ablated with inactivating nuclease mutations (FIGS. 30B-30C). Intriguingly, an autonomous element that also encoded TnpA led to a 50-fold increase in colony counts, and 98% of the surviving colonies were lacZ, indicating a disruption of the target site (FIGS. 30B-30C), and was dependent on the catalytic activity of TnpA. To verify that genomic lacZ disruption resulted from insertion of the plasmid-encoded ISGst2 element, colony PCR and long-read Nanopore sequencing of multiple isolates were performed, which revealed the occurrence of scarless recombination events (FIG. 30D). These results highlight the powerful role of the TnpB nuclease in creating double-stranded breaks, and the ability of TnpA to stimulate site-specific recombination. These findings provide insights into potential biological mechanisms that can be utilized for genome editing and targeted site-specific recombination through the use of double-stranded brakes.

In certain embodiments, the incorporation of IS200/IS605 transposon ends into a pDonor substrate, which also contains additional homology arms at integration sites, facilitates the stimulation of DNA recombination reliant on double-strand breaks (DSBs). This technique can be applied to various cell types, including bacterial cells, plant cells, animal cells, and human cells. For instance, mammalian cells can be transfected with a sequence of interest for DNA insertion, accompanied by TnpA and a DNA nuclease capable of inducing site-specific DSBs, thereby enabling site-specific recombination at the DSB site. The DNA nuclease may comprise CRISPR/Cas effectors (e.g., Cas9 or Cas12), RNA-guided DNA nucleases encoded by insertion sequences (e.g., IscB, IsrB, TnpB, or Fanzor), or homing endonucleases (e.g., ISce-I, ICre-I, HO).

In certain embodiments, the IS200/IS605 transposon ends utilized do not include stop codons and incorporate reading frames or linker sequences (such as glycine-serine linkers). These modifications facilitate the insertion of cargo payloads in-frame, into a target gene of interest, resulting in seamless fusions at the protein level with custom polypeptide sequences encoded by the cargo. Consequently, it becomes feasible to append a sequence of interest to a specific protein within the genome.

### Example 8

### DNA Transposition, RNA Self-Splicing, and RNA-Guided DNA Cleavage by Multi-Functional Transposable Elements

### Protein Detection and Database Curation

TnpB: Database was previously curated as described above. There, homologs of TnpB proteins were comprehensively detected using the H. pylori (HpyTnpB) TnpB amino acid sequence (NCBI Accession: WP_078217163.1) and a G. stearothermophilus TnpB amino acid sequence (NCBI Accession: WP_047817673.1) as seed queries for two independent iterative JackHMMER (HMMER suite v3.3.2) searches against the NR database (retrieved on Jun. 11, 2021), with an inclusion and reporting threshold of 1e-30. The union of the two searches was taken, and proteins that were less than 250 aa were removed to trim partial or fragmented sequences, resulting in a database of 95,731 non-redundant TnpB homologs. Contigs of all putative tnpB loci were retrieved from NCBI for downstream analysis using the Bio. Entrez package.

TnpA_{Y} and TnpA_{S}: For TnpB-associated contigs, TnpA_{Y} was detected using the Pfam Y1_Tnp (PF01797) model for a HMMsearch from the HMMR suite (v3.3.2), with an E-value threshold of 1e-4. This search was performed on the curated CDSs of each contig from NCBI. IS elements that encoded TnpB homologs within 1,000 bp of a detected TnpA_{Y} were defined as autonomous. Analysis of TnpA_{S} association with TnpB was performed with the same methodology mentioned above, but with Pfam serine resolvase (PF00239) model.

Arc-like ORF: A manually identified Arc-like protein (NCBI Accession: WP_003367503.1) was used as the seed query in a two-round PSI-BLAST search against the NR database (retrieved on Aug. 17, 2023). A neighborhood analysis was conducted on ORFs within 10 KB of all detected Arc-like ORF loci using HMMscan from the HMMR suite (v3.3.2) with the Pfam database of HMMs (retrieved on September 2023), and TnpB homologs were specifically searched for using the TnpB-specific models produced from the JackHMMER. High frequency associations with Arc-like ORFs were manually inspected and putative functional associations were manually annotated.

ncRNA Covariation Analyses

Group I Intron: The initial search for group I introns associated with TnpB was performed using the Group I Intron Sequence and Structure Database models of available subclasses, refined by Nawrocki et al. 2018 (Nucleic Acids Res. 2018 Sep. 6; 46 (15): 7970-7976) and Zhou et al. 2008 (Nucleic Acids Res. 2008 January; 36 (Database issue): D31-7). The 14 Group I intron subclass models were searched against all identified TnpB associated contigs with cmscan (Infernal v1.1.4). A liberal minimum bit score of 15 was used to capture distant or degraded introns, and the identification of a putative IStron was supported by its proximity, orientation, and relative location to the nearest identified TnpB ORF. Remaining intron hits were considered associated with TnpB if they were upstream, on the same strand, and within a 1000 bp of a TnpB ORF. After inspecting the database of models, most only captured the catalytic subdomains of the intron and lacked other substructures both 5′ and 3′ of the hit. To address this, the boundaries of the group I intron found to be associated with TnpBs were refined and used to generate a more accurate, comprehensive covariance model. Hits to the models for loci with TnpBs closely related to the C. botulinum TnpB experimentally tested in the study were retrieved. The 1500 bp upstream of those TnpBs were extracted and clustered by 99% length coverage and 99% alignment coverage using CD-HIT to remove identical sequences. The resulting sequences were aligned using MAFFT-EINSI for 8 iterations. The 5′ boundary of the intron (and the LE of the IStron) was manually identified as the boundary of significant drop-off of sequence identity in the alignment. Sequences were subsequently trimmed to that boundary. A structure-based multiple alignment was then performed using mLocARNA (v1.9.1) with the following parameters:
--max-diff-am 25--max-diff 60--min-prob 0.01--indel-50--indel-open-750--plfold-span 100--alifold-consensus-dp

The resulting alignment with structural information was used to generate a new group I intron covariance model with the Infernal suite and refined/verified by R-scape at an E-value threshold of 1e-5. The resulting covariance model was used with cmsearch to discover new group I introns within the curated TnpB associated contig database. The resulting sequences were aligned to generate a new CM model that was used to again search the TnpB-associated contig database. After refinement, the final group I intron CM model was searched against the entire NT database (retrieved on Aug. 29, 2023) with a higher bit-score of 40.

ωRNA: The initial boundaries of the ωRNA associated with the IStron TnpBs were identified as described above. To refine these models to get structures more representative of IStron605 and IStron607 elements, sequences 200 bp downstream and 50 bp upstream of the last nucleotide of the TnpB ORF were extracted to define the RE and transposon boundaries. The ˜150-bp sequences were clustered by 99% length coverage and 99% alignment coverage using CD-HIT to remove duplicates. The remaining sequences were then clustered again by 95% length coverage and 95% alignment coverage using CD-HIT. This was done to identify clusters of sequences that were closely related but not identical, as expected of IS elements that have recently mobilized to new locations. For the 100 largest clusters, which all had a minimum of 10 sequences, MUSCLE (v3.8.1551) with default parameters was used to align each cluster of sequences. Then, each cluster alignment was manually inspected for the boundary between high conservation and low conservation, or where there was a stark drop-off in mean pairwise identity over all sequences. This point was annotated for each cluster as the putative 3′ end of the IS elements. If there was no conservation boundary, sequences in these clusters were expanded by another 150 bp, in order to capture the transposon boundaries, and realigned. The consensus sequence of each alignment (defined by a 50% identity threshold up until the putative 3′ end) was extracted, and rare insertions that introduced gaps in the consensus were manually removed. With the 3′ boundary of the IS element, and thus the 3′ boundary of the TnpB ωRNA properly defined, a covariance model of the TnpB ωRNA could be built.

200-bp window of sequence upstream of the 3′ end for elements of the CboIStron clade and the CdiIStron clade was extracted. A structurally based multiple alignment was then performed using CMfinder and used to generate a TnpB-specific ωRNA covariance model with Infernal and refined/verified with R-scape at an E-value threshold of 1e-5. This was iterated twice to generate the covariation model for each of the two classes of IStrons.

### Phylogenetic Analyses

TnpB and Arc-like ORF: For TnpB found in putative IStron elements, protein sequences were clustered at 95% length coverage and 95% alignment coverage using CD-HIT. The clustered representatives were taken and aligned using MAFFT (v7.508) with the E-INS-I method for 16 rounds. Post-alignment cleaning consisted of using trimA1 (v1.4.rev15) to remove columns containing more than 99% of gaps and manual inspection. The phylogenetic tree was created using IQ-Tree 2 (v2.1.4) with a model of substitution identified using ModelFinder, and optimized trees with nearest neighbor interchange to minimize model violations. Branch support was evaluated with 1000 replicates of SH-aLRT, aBayes, and ultrafast bootstrap support from the IQTREE package. The tree with the highest maximum likelihood was used as the reconstruction of the IStron TnpB phylogeny.

All Arc-like ORF hits were aligned using MAFFT (v7.508) with the E-INS-I method for 8 rounds. The rest of the analysis was identically performed as above.

Group I Introns: For all the group I intron hits from the search against the NT database, hits smaller than 300 bp were removed. The remaining sequences were clustered at 90% length coverage and 90% alignment coverage using CD-HIT. The clustered representatives were taken and aligned using MAFFT (v7.508) with the E-INS-I method for 2 rounds. Post-alignment cleaning consisted of using trimA1 (v1.4.rev15) to remove columns containing more than 99% of gaps and manual inspection. The phylogenetic tree was created using IQ-Tree 2 (v2.1.4) with a model of substitution identified using ModelFinder, and optimized trees with nearest neighbor interchange to minimize model violations. Branch support was evaluated with 1000 replicates of SH-aLRT, aBayes, and ultrafast bootstrap support from the IQTREE package. The tree with the highest maximum likelihood was used as the reconstruction of the group I intron phylogeny. Neighborhood analysis was performed similarly to how the Arc-like ORFs were analyzed.

Culturing of Clostridia senegalense

A Clostridia strain encoding IStrons with similarity of ˜80% to CboIStron was obtained from ATCC (strain 25772), where it was defined as belong to an unknown species classification. Internal rRNA phylogenetic analysis led to the assignment of this strain as a member of species senegalense. Clostridia senegalense was cultured from a lyophilized ATCC pellet in 5 mL of Gifu Anaerobic Medium Broth, Modified (mGAM; HyServe, 05433) under anaerobic conditions (5% H₂, 10% CO₂ and 85% N₂) in an anaerobic chamber. All media was pre-reduced for ˜24 h before use in culturing. C. senegalense was then banked as a glycerol stock (final concentration 20%) and sub-cultured into 100 mL cultures of mGAM. The growth of these cultures was monitored with a spectrophotometer over ˜6 h until a final OD600 of 0.4-0.6 (exponential phase), at which point cultures were poured into two 50 mL falcon tubes and cooled on ice for 10 minutes. The cultures were then centrifuged at 4,000 g for 10 minutes at 4° C., supernatant decanted, and cell pellets flash frozen in liquid nitrogen. Pellets were stored at −80° C. until RNA extraction and processing.

### RNA Extraction

RNA from the Clostridia senegalense cell pellets were extracted in 96-well format using a silica bead beating-based protocol adapted from a prior study. Briefly, 200 μl 0.1 mm Zirconia Silica beads (Biospec, 11079101Z) were added to each well of 96-well deep-well plates (Thermo Fisher Scientific, 07-202-505). Next, cell pellets were resuspended in 500 μL DNA/RNA shield buffer (Zymo) and transferred to each well and the plates were affixed with a scaling mat (Axygen, AM-384-DW-SQ) and centrifuged for 1 minute at 4,500 g. To avoid overheating during bead beating, the plates were vortexed for 5 seconds and incubated at −20° C. for 10 minutes before beating. Then, plates were fixed on a bead beater (Biospec, 1001) and subjected to bead beating for 5 minutes, followed by a 10 minute cooling period. The bead beating cycle was repeated three times total and plates were the centrifuged at 4,500×g for 5 minutes to spin down cell debris. Next, 60% of the bead beating volume was transferred to the Zymo Quick-RNA Miniprep Plus kit (Cat. No. R1057) and RNA was purified using the manufacturer's protocol for gram positive bacteria. RNA quality was assessed using the 260/280 nm ratio (˜2.0) as measured by Nanodrop (Cat. No.) and concentration was measured by the Qubit RNA High Sensitivity Assay Kit (Cat. No. Q32852) using the manufacturer's protocol. RNA was stored at −80° C. until library preparation.

### Total RNA and Small-RNA Sequencing

For total RNA-seq library preparation, 10 μg of purified RNA was treated with Turbo DNase I (Thermo Fisher Scientific) for 1 h at 37° C. using the manufacturer's protocol. A 2× volume of Mag-Bind TotalPure NGS magnetic beads (Omega) was added to each sample and the RNA was purified using the manufacturer's protocol. The RNA was then diluted in NEBuffer 2 (NEB) and fragmented by incubating at 92° C. for 1.5 minutes. To generate RNA with 5′ monophosphate and 3′ hydroxyl ends, samples were treated with RppH (NEB) supplemented with SUPERase. In RNase Inhibitor (Thermo Fisher Scientific) for 30 min at 37° C., followed by T4 PNK (NEB) in 1× T4 DNA ligase buffer (NEB) for 30 min at 37° C. Samples were column-purified using RNA Clean & Concentrator-5 (Zymo) and the concentration was determined using the DeNovix RNA Assay.

For sRNA-seq, a protocol was adapted for Clostridia senegalense from a prior study. 10 μg of purified RNA and 1 μg of Century+ RNA markers (Invitrogen, Cat. No. AM7145) were first mixed with 2×RNA loading dye at a 1:1 ratio and heat denatured at 95° C. for 5 minutes. Next, the samples were loaded into separate wells of a pre-cast 5% denaturing urea polyacrylamide gel and run at 250 V for 45 minutes in 1×TBE at 4° C. until the bromophenol blue dye front ran off the gel. Gels were stained with 20 μL of SybrGold dye and 1×TBE on a rotator for 5 minutes. Gels were then visualized on a blue light box and bands ranging from just below 100 bp and just above 500 bp were excised using a fresh razor blade and transferred to a 2 mL centrifuge tube. 0.3 M NaCl was added to the gel slices, vortexed, and left rotating overnight at 4° C. The next day, the tubes were centrifuged at 17,000 g for 1 minute to collect tiny gel slices to the bottom of the tube and the supernatant was transferred into three fresh 1.5 mL centrifuge tubes with 340 μL each. 1 μL of GlycoBlue was added to each tube and vortex, followed by addition of 3 volumes of 100% ethanol to each tube and incubation on ice for 1 h. The tubes were then centrifuged at 17,000 g for 15 minutes at 4° C., vortexed for 10 seconds to strip precipitates, and centrifuged for another 15 minutes at 4° C. Supernatant was gently removed with a pipet to avoid the pellet and 900 μL of ice-cold 75% ethanol was added, followed by a brief vortex and centrifugation at 17,000 g for 5 minutes at 4° C. This was repeated for 2 washes in total. After removal of residual ethanol, RNA pellets were air-dried for 10 minutes at room temperature and dissolved in 20 μL of nuclease-free ultra-pure water. Samples were immediately put on ice or stored at −80° C. 1 μg of purified small RNA was then treated with Turbo DNase I (ThermoFisher Cat. No. AM2238) for 1 h at 37° C. using the manufacturer's protocol. 2× volume of Mag-Bind TotalPure NGS magnetic beads (Omega) were added to each sample and the RNA was purified using the manufacturer's protocol. End repair was performed as described above for total RNA-seq libraries.

For both total RNA and small RNA samples, Illumina adapter ligation and cDNA synthesis were performed using the NEBNext Small RNA Library Prep kit. Dual index barcodes were added by PCR amplification (12 cycles), and the cDNA libraries were purified using the Monarch PCR & DNA Cleanup Kit (NEB). High-throughput sequencing was performed on an Illumina NextSeq 550 in paired-end mode with 150 cycles per end.

Whole Genome Sequencing of Clostridia senegalense

Genomic DNA from Clostridia senegalense was extracted using the Promega Wizard Genomic DNA purification kit, following the manufacturer's protocol for gram-positive bacteria. DNA was measured by fluorescent quantification. TnY, a homolog of Tn5, was purified in-house following previous methods. 10 ng of purified gDNA was tagmented with TnY preloaded with Nextera Read 1 and Read 2 oligos, followed by proteinase K treatment (NEB, final concentration 16 units per mL) and column purification. PCR amplification and Illumina barcoding was done for 13 cycles with KAPA HiFi Hotstart ReadyMix; the PCR reaction was then resolved on a gel, and a smear from 400 bp to 800 bp was extracted for sequencing on a paired end, 150×150 NextSeq kit. Downstream analysis was performed as described in total RNA sequencing. De novo genome assembly was also performed by Plasmidsaurus, and the assembled genome was in agreement with the 4 Mbp genome provided for ATCC 25772.

### Targeted Tagmentation-Based Detection of IS Excision Events

100 ng of purified gDNA of Clostridia senegalense was tagmented with TnY preloaded with full-length Nextera Read 2/Indexed oligos. An initial PCR amplification was done with a forward oligo that anneals in the upstream genomic sequence flanking the IStron and an oligo that anneals to the P7 sequence using KAPA HiFi Hotstart with an annealing temperature of 55° C. and 1 minute extension time. After bead cleanup using Omega Mag-Bind TotalPure magnetic beads at a ratio of 0.9×, a second PCR was done with an oligo that annealed to the initial PCR amplicon within ˜40 bp of the genomic-IStron junction. This forward oligo had all necessary sequences for Illumina sequencing. After 15 cycles of PCR under the same conditions, the reaction was resolved on a gel, and a smear from 350 bp to 800 bp was extracted for sequencing with at least 75 Read 1 cycles. After adapter trimming, the relative abundance of reads that contain a 20 bp sequence of the IStron end or contain a 20 bp sequence of the downstream genomic sequence were tallied using BBDuk from the BBTools suite (v.38.00; sourceforge.net/projects/bbmap) with a hamming distance of 2 and an average Qscore greater than 20.

### RNA-Sequencing Analyses

RNA-seq data were processed using cutadapt v4.2 to remove adapter sequences, trim low-quality ends from reads, and exclude reads shorter than 18 bp. Reads were mapped to the reference genome (Cdi: NZ_CP010905.2; Cse: ATCC 25772) using the splice-aware aligner STAR v2.7.10, with --outFilterMultimapNmax 10. Mapped reads were sorted and indexed using SAMtools v1.17. Splice junctions inferred by STAR flanking loci of interest were used to create a custom genome annotation file for a second round of STAR alignment in order to refine spliced read counts. Sashimi plots showing read coverage and spliced reads at specific loci were generated with ggsashimi v1.1.5 in strand-specific mode. To quantify splicing activity at each intron locus, reads were mapped to a mock reference sequence spanning either the 5′ exon-intron junction, 3′ exon-intron junction, or the exon-exon junction. Reads mapping to each junction were quantified using featureCounts v2.0.2, with a minimum overlap of 3 bp on either end of the junction. Splicing activity was calculated as the number of reads mapping to the exon-exon junction divided by the average of reads mapping to the exon-intron junctions.

### RIP-Seq

E. coli str. K-12 substr. MG1655 (sSL0810) was transformed with 3×FLAG-CboTnpB (pSL5412) or 3×FLAG-CboTnpB (D189A) (pSL5413) and ωRNA encoding plasmids. Single colonies were inoculated in liquid LB with spectinomycin (100 μg ml⁻¹) and grown overnight. Next day the culture was inoculated at 100× dilution in 50 ml of liquid LB with spectinomycin (100 μg ml⁻¹) and grown until OD600 reached 0.5. 10 ml of culture centrifuged at 4,000 g for 10 min at 4° C. and supernatant removed. The pellet was washed once with 1 ml of cold TBS, centrifuged at 10,000 g for 5 min at 4° C., supernatant removed and resulting pellet flash-frozen in liquid nitrogen. Pellets stored at −80° C. Antibodies for immunoprecipitation were conjugated to magnetic beads as follows: for each sample, 30 μl Dynabeads Protein G (Thermo Fisher Scientific) was washed 3× in 1 ml RIP lysis buffer (20 mM Tris-HCl pH 7.5, 150 mM KCl, 1 mM MgCl₂, 0.2% Triton X-100), resuspended in 1 ml RIP lysis buffer, combined with 10 μl anti-FLAG M2 antibody, and rotated for >3 h at 4° C. Antibody-bead complexes were washed an additional 3× to remove unconjugated antibodies, and were resuspended in 30 μl RIP lysis buffer per sample.

To generate cell lysates, flash-frozen pellets were first resuspended in 1.2 ml RIP lysis buffer supplemented with complete Protease Inhibitor Cocktail (Roche) and SUPERase·In RNase Inhibitor (Thermo Fisher Scientific). Cells were then sonicated for 1.5 min total (2 sec ON, 5 sec OFF) at 20% amplitude. To clear cell debris and insoluble material, lysates were centrifuged for 15 min at 4° C. at 21,000×g, and the supernatant was transferred to a new tube. At this point, a small volume of each sample (24 μl, or 2%) was set aside as the “input” starting material and stored at −80° C.

For immunoprecipitation, each sample was combined with 30 μl antibody-bead complex and rotated overnight at 4° C. The next day, each sample was washed 3× with ice-cold RIP wash buffer (20 mM Tris-HCl pH 7.5, 150 mM KCl, 1 mM MgCl₂). After the last wash, beads were resuspended in 1 ml TRIzol (Thermo Fisher Scientific) and incubated at RT for 5 min to allow separation of RNA from the beads. A magnetic rack was used to isolate the supernatant, which was transferred to a new tube and combined with 200 μl chloroform. Each sample was mixed vigorously by inversion, incubated at RT for 3 min, and centrifuged for 15 min at 4° C. at 12,000 g. RNA was isolated from the upper aqueous phase using the RNA Clean & Concentrator-5 kit (Zymo), eluting in 15 μl RNase-free water. RNA from input samples was isolated in the same manner using TRIzol and column purification.

For RIP-seq library preparation (input and RIP eluates), 6 μl RNA was diluted in FastAP Buffer (Thermo Fisher Scientific) supplemented with SUPERase. In RNase Inhibitor (Thermo Fisher Scientific) to a total volume of 18 μl, and fragmented by heating to 92° C. for 1.5 minutes. Each sample was treated with 2 μl TURBO DNase for 30 min at 37° C. and column-purified using the RNA Clean & Concentrator-5 kit (Zymo), eluting in 12.5 μl RNase-free water. RNA concentration was quantified using the DeNovix RNA Assay. Illumina sequencing libraries were prepared using the NEBNext Small RNA Library Prep kit, and libraries were sequenced on an Illumina NextSeq 500 in paired-end mode with 75 cycles per end.

Plasmid and E. coli Strain Construction

Genes encoding CboTnpA and native CboIStron sequence were synthesized by Twist Bioscience. E. coli codon optimized ChoTnpB and bioinformatically predicted ωRNA were synthesized and cloned into a single pCDF-duct vector by Genscript, with two separate J-23 series promoters driving their expression. Transposase expression plasmids were generated using Gibson assembly, by inserting TnpA gene downstream of pLac or T7 promoters in minimal pCOLADuet-1 vector constructed as above. Native IStron, IStron with TnpB only and mini-IS sequence (581 bp from the right end and 221 bp of the right end) were cloned using Gibson assembly, by inserting them into a pCDF-duet vector downstream of T7 promoter. pTarget plasmids were generated by around-the-horn PCR, inserting 44-bp a target sequence into a minimal pCOLDA-Duet-1 vector. Transposition intermediate (pDonorCI) was generated by Gibson assembly of CboIStron left end (581 bp), right end (221 bp), R6K ori and chloramphenicol resistance gene. Cloning mix was transformed to pir+ strain, to allow for the propagation of R6K ori bearing plasmid. Derivatives of these plasmids were cloned using a combination of methods, including Gibson assembly, restriction digestion-ligation, ligation of hybridized oligonucleotides, Golden Gate Assembly and around-the-horn PCR. Plasmids were cloned, propagated in NEB Turbo cells (NEB) (except for pCircInt derivatives, which were propagated in pir+ strain), purified using Miniprep Kits (Qiagen), and verified by Sanger sequencing (GENEWIZ).

DNA Cleavage Assays with TnpB

Plasmid interference assays were performed in E. coli str. K-12 substr. MG1655 (sSL0810) when synthetic ChoTnpB expression construct was used, and in E. coli BL21 (DE3) strain for all other experiments. When ChoTnpB was co-expressed with ωRNA from the same plasmid, BL21 (DE3) cells were transformed with a pEffector plasmid, and single colony isolates were selected to prepare chemically competent cells. 200 ng of pTarget plasmid were then delivered via transformation. After 2 h, cells were spun down at 6000 rpm for 5 min and resuspended in 30 μl of LB. Cells were then serially diluted (10×) and plated on LB agar media containing spectinomycin (100 μg ml⁻¹) and kanamycin (50 μg ml⁻¹) and grown for 24 h at 37° C. Plates were imaged in an Amersham Imager 600. For the experiments when mini-IS was used as a guide for ChoTnpB, BL21 (DE3) cells were co-transformed with mini-IS and TnpB expression plasmids, and single colony isolates were selected to prepare chemically competent cells. Second transformation was performed as indicated previously, and cells were plated on LB agar media containing spectinomycin (100 μg ml⁻¹), chloramphenicol (25 μg ml⁻¹), kanamycin (50 μg ml⁻¹) and IPTG (0.1 mM) and grown for 24 h at 37° C. Plates were imaged in an Amersham Imager 600.

### TAM Library Experiments and Analyses

TAM library experiments were prepared for sequencing as previously described.

Analysis was performed as previously described; in brief, reads were filtered on containing the correct sequence both upstream and downstream of the TAM region. TAM sequences were then extracted, tallied, and depletion values were calculated as the relative abundance of the library member in the input library divided by the relative abundance of the library member in the output. Sequence logos were generated with the library members that were depleted more than 5-fold (depletion value greater than 32) using WebLogo (v2.8), and the top 5% of depleted library members were used to generate TAM wheels.

Transposon Excision Assays with TnpA_{S}

For each excision assay, E. coli str. K-12 substr. MG1655 was transformed with TnpA expression plasmid and selectively grown on LB with kanamycin (50 μg ml⁻¹). A single colony was used to make chemically competent cells, which were then transformed with 100 ng mini-IS element encoding plasmid. Cultures were grown overnight at 37° C. on LB-agar with spectinomycin (100 μg ml⁻¹), kanamycin (50 μg ml⁻¹) and IPTG (0.5 mM) for TnpA induction. Scraped colonies were resuspended in LB medium. Approximately 3.2× 10⁸ cells (equivalent to 200 μl of cultures with an optical density at 600 nm (OD600)=2.0) were transferred to a 96-well plate. Cells were pelleted by centrifugation at 4,000 g for 5 min and resuspended in 80 μl of H₂O. Next, cells were lysed by incubating at 95° C. for 10 min in a thermal cycler. The cell debris was pelleted by centrifugation at 4,000 g for 5 min, and 10 μl of lysate supernatant was removed and serially diluted with 90 μl of H₂O to generate 10- and 100-fold lysate dilutions for PCR and qPCR analyses, respectively.

Transposon Integration Assays with TnpA_{S}

Plasmids with an R6K ori, a CmR marker, and inverted IStron ends (pDonorCI) were cloned in pir+ strains. E. coli str. K-12 substr. MG1655 was transformed with a pLac-TnpA expression plasmid and various pDonorCI variants via electroporation, recovered for 7 hours, plated on LB Agar plates with chloramphenicol, and grown for ˜24 hours. Surviving colonies were pooled and genomic DNA was extracted and quantified via Qubit. Approximately 100 ng of gDNA was tagmented with TnY, pre-loaded with Read 2 Nextera oligos. 2 rounds of PCR were performed as described for targeted detection of IS excision events with oligos that annealed to either the left or right IStron end. Paired-end, 76×76 cycle sequencing was performed on a NextSeq platform. Using BBDuk, reads were then filtered for containing the proper IStron end sequence, and the flanking genomic sequence was extracted. Reads that contained the parental pDonorCI sequence were removed during this process. Flanking genomic sequences were then aligned to the E. coli genome using Bowtie2. WebLogo representations were then generated using the input sequence on both the left and right IStron end, as well as the mapped genomic insertion sites.

Transposon Maintenance Experiments with TnpA_{S} and TnpB

sSL3391, a derivative of E. coli str. K-12 substr. MG1655 with a lacZ deletion replaced by a chloramphenicol resistance cassette, was transformed with 400 ng of plasmid encoding an intact lacZ gene (pSL4825, empty vector) or a CboIStron-interrupted lacZ gene (pSL5948, pSL5949, pSL5950). Following transformation, colonies were plated on MacConkey agar media containing tetracycline (10 μg ml⁻¹) to enrich for IStron excision events. Cells were grown at 37° C. for 36 hours, then harvested, serially diluted, and plated onto LB agar containing tetracycline (10 μg ml⁻¹) and X-gal (200 μg ml⁻¹) and grown for 18 h at 37° C. Total number of colonies were counted, along with the number of blue colonies to determine the frequency of excision and reintegration events. In addition, genomic lysate was harvested from cells as described above for PCR analysis.

Transposon Recombination Assay with TnpA_{S} and TnpB

E. coli str. K-12 substr. MG1655 (sSL0810) containing an intact lacZ loci were chemically transformed with 400 ng of plasmid encoding an intact lacZ gene (pSL4825, empty vector) or CboIStron-interrupted lacZ gene (pSL5948, pSL5949, pSL5950), recovered for 1 h at 37° C. in liquid LB, and serially diluted on LB-agar plates with tetracycline (10 μg ml⁻¹). Next day colonies were counted and converted to CFUs per μg of DNA. Tetracycline plates were then replica plated to LB-agar plates containing both tetracycline (10 μg ml⁻¹) and X-gal (200 μg ml⁻¹) for blue/white colony screening. White colonies were counted to determine the frequency of recombination events at the genomic lacZ locus.

### In Vitro Splicing Assays

Templates for in vitro splicing reactions were obtained by PCR amplification of Marker (mock excised), splicing mutant and mini-IS containing plasmids. All templates had a T7 promoter encoded within the plasmid, which is required for transcription. PCR products were extracted from gel and 1 μg of each was used in 50 μl in vitro transcription reaction. Reactions were set up in 30 mM Tris (pH 8.0 at 25° C.), 10 mM DTT, 0.1% Triton X-100, 0.1% spermidine, 60 mM MgCl₂, 0.2 μl SUPERase In™ (Thermo Fisher Scientific), 6 mM each NTP and 0.2 mg/ml of T7 polymerase containing buffer. Reactions were incubated overnight at 37° C. Next day, pyrophosphate precipitate was removed by centrifugation and DNA template digested by adding 1 μl of TURBO™ DNase (2 U/μL) (Thermo Fisher Scientific) and incubating for 30 min at 37° C. Resulting RNA was purified using the NEB Monarch RNA Cleanup Kit. Purified RNA was stored at −80° C.

### In Vivo Splicing Assays

In vivo splicing assays were performed in E. coli BL21 (DE3) strain transformed with mini-IS variant encoding plasmid, or co-transformed with mini-IS and TnpB expression plasmids. For single plasmid transformations, single colonies were picked from a plate and inoculated to grow overnight in LB with spectinomycin (100 μg ml⁻¹). In the morning, the cultures were re-inoculated at 40× dilution in LB supplemented with spectinomycin (100 μg ml⁻¹) and IPTG (0.1 mM), and grown until OD600 reached 0.5-0.7. Then an aliquot equivalent to 250 μl of cell suspension at OD600 was taken from each culture, centrifuged at 6000 rpm for 5 min and cell pellet resuspended in 750 μl Trizol (Thermo Fisher Scientific). After incubating 10 min at room temperature 150 μl of chloroform was added, tubes shaken and centrifuged at 12,000 g for 15 min at 4° C. Aqueous phase was transferred to a new tube and mixed with equal volume of absolute ethanol (>96%), following RNA purification using the NEB Monarch RNA Cleanup Kit. Purified RNA was stored at −80° C. For splicing assays with TnpB co-expressed in trans, single colonies were inoculated to grow overnight in LB with spectinomycin (100 μg ml⁻¹) and chloramphenicol (25 μg ml⁻¹). In the morning, the cultures were re-inoculated at 40× dilution in LB supplemented with spectinomycin (100 μg ml⁻¹), chloramphenicol (25 μg ml⁻¹) and IPTG (0.5 mM), and grown until OD600 reached 0.5-0.7. All downstream steps were performed as described before.

### Reverse Transcription

200 ng of the purified total RNA was used as an input for reverse transcription reaction. First, total RNA was treated with 1 μl dsDNase (Thermo Fisher Scientific) in 1× dsDNase reaction buffer in the final 10 μl volume, incubating at 37° C. for 20 min. Then 1 μl of 10 mM dNTP, 1 μl of 2 mM IStron-interrupted gene-specific primer and 1 μl of 2 mM SpecR specific primer were added for gene-specific priming and reactions were heated at 65° C. for 5 min. Incubation was stopped by placing the tubes directly on ice, followed by addition of 4 μl of SSIV buffer, 1 μl 100 mM DTT, 1 μl SUPERase In™ (Thermo Fisher Scientific) and 1 μl of SuperScript IV Reverse Transcriptase (200 U/μl, Thermo Fisher Scientific) and incubation at 53° C. for 10 min and 80° C. for 10 min. The resulting cDNA was diluted and used for end-point or quantitative PCR. Endpoint PCR was performed in a 20 μl reaction volume containing 1× OneTaq Master Mix (NEB), 0.2 μM of each primer and 1 μl of 100-fold diluted cDNA. Thermal cycling: DNA denaturation (94° C. for 30 s), 30 cycles of amplification (denaturation: 95° C. for 15 s, annealing: 46° C. for 15 s, extension: 68° C. for 15 s), followed by a final extension (68° C. for 5 min). Products were resolved by 1.5% agarose gel electrophoresis and visualized by staining with SYBR Safe (Thermo Fisher Scientific). Quantitative PCR was performed in 10 μl reaction containing 5 μl SsoAdvanced™ Universal SYBR Green Supermix (BioRad), 1 μl H₂0, 2 μl of primer pair at 2.5 μM concentration and 2 μl of 100-fold diluted lysate (10-fold when intron was expressed from a J23114 promoter). Two primer pairs were used: (1) spliced RNAs were captured using a forward primer annealing to exon1 and reverse primer spanning the splice-junction; (2) unspliced products were amplified using the same forward primer annealing to exon1 and reverse primer annealing to IStron left end. Reactions were prepared in 384-well clear/white PCR plates (BioRad), and measurements were performed on a CFX384 RealTime PCR Detection System (BioRad) using the following thermal cycling parameters: polymerase activation and DNA denaturation (98° C. for 2.5 min), 40 cycles of amplification (98° C. for 10 s, 62° C. for 20 s), and terminal melt-curve analysis (decrease from 95° C. to 65° C. in 0.5° C./5 s increments). For each sample, the ratio of spliced/unspliced was obtained by calculating spliced/unspliced=2^{-ΔCq}, where ΔCq=Cq(spliced)−Cq(unspliced).

Some TnpA and TnpB homologs are encoded within group I introns, generating chimeric genetic elements called IStrons. These elements are not only mobile on the DNA level, due to TnpA and TnpB, but are phenotypically silent on the RNA level because the whole element is removed during splicing. IStrons can harbor TnpA and TnpB proteins related to either IS605 or IS607, suggesting multiple IS element acquisition events by group I intron during evolution. Some of the IStrons encoding proteins from IS607 elements were found in pathogenic bacteria species of Clostridium botulinum. Under low-oxygen conditions these bacteria produce highly dangerous toxins that block nerves and cause muscle and nerve paralysis. An IStron homolog from this species showed that TnpB (ChoTnpB) is active for double-stranded DNA cleavage in E. coli. TnpB from IS607 elements cleaves DNA when both TAM and target-complementary ωRNA guide are present and this activity is dependent on its RuvC active site. The same active site is also responsible for ωRNA maturation on the 5′ end. Transposase (CboTnpA) associated with this TnpB recognizes CboIStron ends and can excise the element from its native location. Lastly, the CboIStron can self-splice from the E. coli RNA transcript.

TnpA derived from IS607-family transposons represents a serine-family recombinase, hereby indicated by the suffix “(S)” to signify its serine catalytic active site. Contrarily, the previously published Meers work on TnpA corresponds to a tyrosine-family recombinase, distinctly referenced as TnpA (Y), emphasizing its tyrosine catalytic active site. These designations, “(S)” and “(Y)”, underscore the differentiation between these enzyme families or classes of transposons.

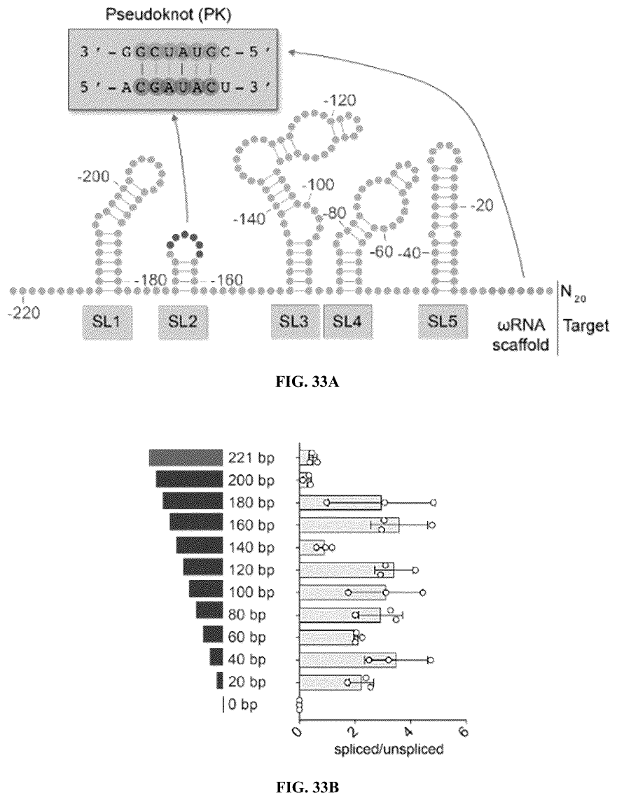

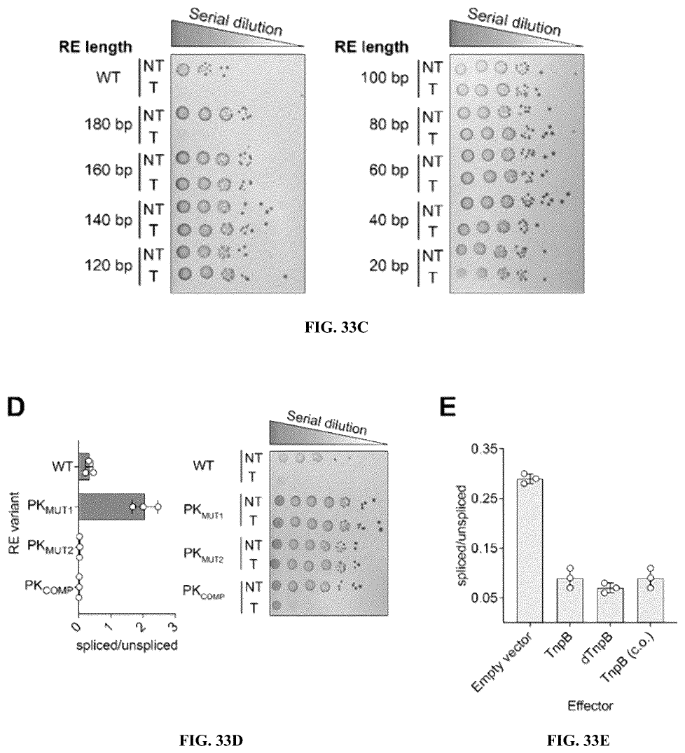

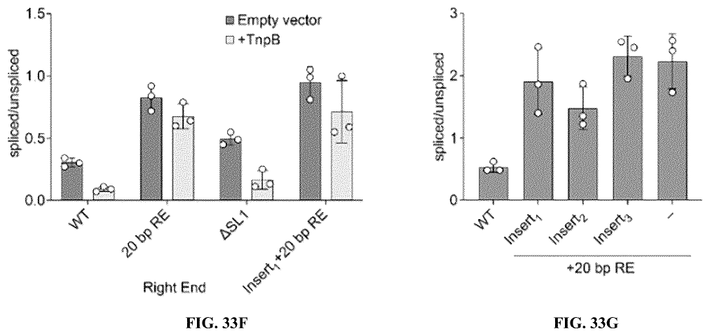

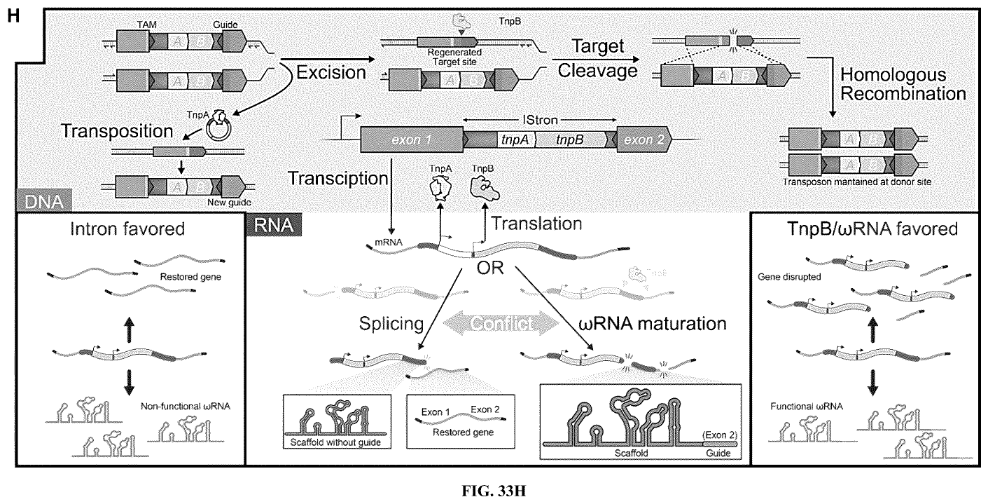

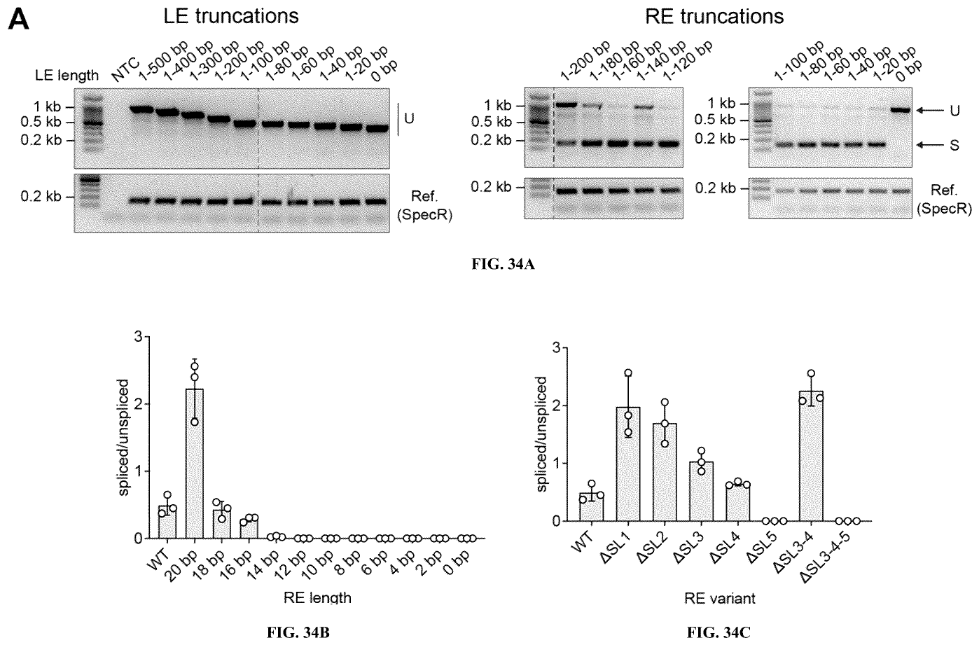

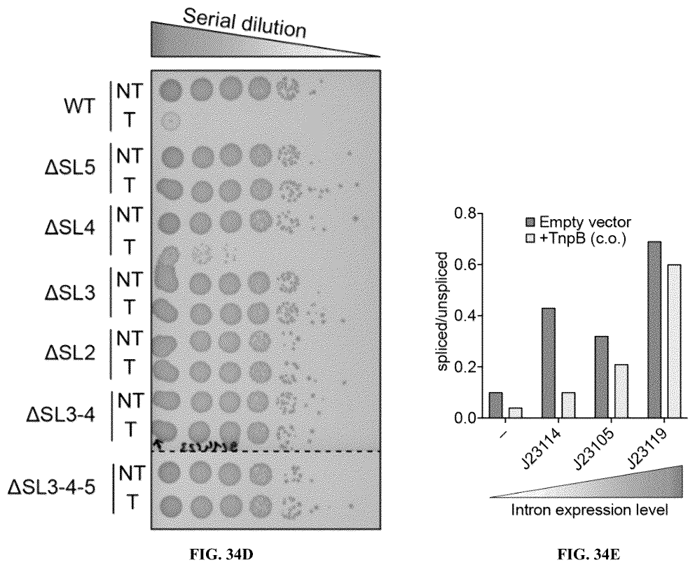

Herein the minimal TnpB ωRNA sequence was defined and some primary sequence elements can be changed while preserving the structural fold of the RNA (e.g., complementary mutations for the pseudo-knot shown in FIG. 33D). Some structural features of ωRNA can be removed (e.g., FIG. 34D, removal of SL4) to attenuate ChoTnpB activity, suggesting that alterations to ωRNA can be made to modulate TnpB activity.

TnpB derived from C. botulinum originates from the IS607-family elements. IS607-family elements represent a distinct evolutionary lineage, separate from the IS200/IS605-family transposons.

In some embodiments, RNA splicing activity can be repressed in the presence of TnpB. Different intron sequence elements differ in their susceptibility for TnpB repression (FIG. 33F). It could be possible to have multiple similar copies of the same element in a cell or genome, which would differ only in their right-end encoded ωRNA portion, which is recognized by TnpB. Only the IStron elements that have a TnpB-binding competent ωRNA would be expected to be recognized and their splicing selectively repressed by TnpB.

IStrons may serve as platforms for introducing selection markers, facilitating their placement within any gene, even those categorized as essential. As evidenced, IStrons can splice at the RNA level, resembling the characteristics of group I introns. When DNA segments containing drug markers are situated within the IStron boundaries, encompassing both the left and right ends crucial for excision and splicing, a seamless genomic integration is achieved, ensuring the original function of the host gene remains undisturbed. This enables the expression of the drug marker, facilitating selection, while concurrently ensuring that RNA splicing remains unaffected, thus preserving the unaltered function of the gene in question. Upon the need for marker elimination, TnpA is engaged. Acting on the exact boundaries used for RNA splicing, it guarantees a precise, scarless excision, while preserving the flanking sequences intact during IStron integration. Moreover, if the element is inserted using HDR, the mutations resulting from the IStron incorporation can be stably integrated into these flanking sequences, providing a platform for modifying essential genes with selection capabilities.

The IS element may be characterized by the encoding of TnpB, which may be in association with TnpA (Y), TnpA(S), or independently without either of these TnpA variants. Additionally, a predetermined gene of interest may be embedded within the confines of the said IS element. Integral to the structure of the IS element is the ωRNA sequence, strategically located at its right end, designed such that it autonomously derives its guide sequence from its adjacent genomic environment.

The IS element can be seamlessly integrated into a wide spectrum of heterologous genomes, encompassing, but not limited to, bacteria, fungi, insects, and mammals, employing conventional genome editing techniques. Once integrated, the IS element adopts the role of an adaptive ‘gene drive’. This process is aided by the TnpB or IscB, which, in complex with ωRNA, utilize its intrinsic ability to initiate homologous recombination. This targets native sequences on either sister or homologous chromosomes, particularly those without the IS element.

Should TnpA orchestrate the relocation of the IS element within a different genomic locale, TnpB is equipped to spontaneously adapt and secure a novel guide for the ωRNA, ensuring its sustained function in the new setting. This mechanism stands in contrast to the established Cas9-centric gene drive methodologies which necessitate a statically pre-defined sgRNA for locus-specific targeting. Such traditional sgRNAs lack the flexibility to adjust if their corresponding element relocates. In contrast, the dynamic nature of TnpB/IscB-centric gene drives equips them with the adaptability to align with the immediate changes in their genomic surroundings.

TABLE 1

| Nuclease | NCBI Accession # | Protein Sequence | SEQ ID NO |
|---|---|---|---|
| GstTnpB1 | WP_160268933.1 | MANKAYQFRLYPTKEQEQLLAKTFGCVRFVYNKMLEERIQMFE | 1 |
| (encoded | | KFKDDQESLKQQTCPTPAKYKKEFPWLKEVDSLALANAQLNLQ | |
| by | | KAFQHFFSGRAGFPKFKNRKAKQSYTTNMVNGNIKLSDGYIKLP | |
| ISGst1) | | KLKWIKLKQHREIPAHHIIKSCTITKTKTGKYYISILTEYEHQPAP | |
| | | KEVQTVVGLDFSMSTLYVDSEGKRANYPRFYRKALETLAKEQR | |
| | | KLSRKKKGSNRWHKQRLKVAKLHEKIANQRKDFLHKESHKLA | |
| | | KRYDCVVIEDLNMKGMSQALHFGQGVHDNGWGMFTTFLQYK | |
| | | LAEQGKKLIKIDKWFPSSKTCSCCGRVKESLSLSERTFRCECRFE | |
| | | SDRDVNAAINIKHEGMKRLAIV | |
| GstTnpB2 | WP_047817673.1 | MYFCIKQQLNGLTKEEYLTLRELCHIAKNMYNVGLYNVRQYYF | 2 |
| (encoded | | EHKEFLNYEKNYHLAKTNENYKLLNSNMAQQILKKVNEAFKSF | |
| by | | FGLISLAKQGKYDHKAISIPKYLKKDGFHSLIIGQIRIDGNKFTIPY | |
| ISGst2) | | SRLFKKTHKPITITIPPVLLDKKIKQIEIIPKHHARFFEIQYKYEMPE | |
| | | DQRELNDQKALAIDLGLNNLATCVTSDGRSFIIDGRRLKSINQWF | |
| | | NKENARLQSIKDKQKIKGTTRKQALLAMNRNNKVNDYINKTCR | |
| | | YIINYCIENQIGKLVIGYAETWQRNMNLGKKTNQNFVNIPLGNIK | |
| | | EKLEYLCEFYGIEFLKQEESYTSQASFFDGDEIPEYNADNPKEYK | |
| | | FSGKRIKRGLYRTKSGKLINADVNGALNILKKSKAVDLSVLCSS | |
| | | GEVDTPQRIRIA | |
| GstTnpB3 | WP_011231306.1 | MYRTLKTRFRAKKEVIQKLFECNRISAEVWNECLRLAKEHHLET | 3 |
| (encoded | | GKWITKTELQKATKGRFSIHSQSIQAVVHKYIFARDGAKEARKK | |
| by | | GEKIKYPYKKKKHFNTKWAKDGFVLHEDGTLELSLGIWNRKRQ | |
| ISGst3) | | SPLVVKIDKEKLPNGKVKEIELVYDRGLWLCLSYEDGKKPKENP | |
| | | CKNRVAIDPGEIHTIAAICENGESLIITGRKIRSIHRLRNKKLKELQ | |
| | | KLMSRCKKGSKQWKKYNRAKQYVLSKSEAQLKDALHKTTRQF | |
| | | VRWCLENQVKEVVIGDIEGIQRNTKKKRNKKTNQKLSNWSFGK | |
| | | LFDYLKYKLNAEGIQIEKKDESYTSQTCPVCGKKNKSSSRNYTC | |
| | | QCGYKRHRDIHGAMNLFAKVYYGEIRPLEFTVKPFTYRRIA | |
| GstTnpB4 | WP_011229875.1 | MDMTLTAKIKIYPTAEQAEVLKATLSAYRQACNAVSVVIFDTKV | 4 |
| (encoded | | LAQAKLHDMTYRLLRSNYALRSQMAQSVIKTVIARYRSLKSNG | |
| by | | HEWTLVRFKKPEYDLVWNRDYSIVQGLFSVNTLEGRIKVSFEPK | |
| ISGst4) | | GMEPYFDGSWTFGTAKLVYKHNKFFLHIPMTKTIPTVDEHNIRQ | |
| | | VVGVDVGVNFLAVAYDSQGKTIFFNGRKIKHMRAKYKRMRKT | |
| | | LQQKGTASARRKLKTIGQRENRWMTDVNHAVTKALVRQYGER | |
| | | TLFVLEDLTGIREKTERVRIHDRYETVSWAFYQFRQMLEYKARL | |
| | | HGSKVIVVAPHYTSLTCPKCGHTEKANRKKRTHTFCCRTCGYTS | |
| | | NDDRIGAMNLQRKGIEYIVEGTTQA | |
| GstIscB | WP_223812651.1 | MYIINKHGKPLMPCSPRKARLLLKQKKAKVVRRTPFTIQLLYGS | 5 |
| (encoded | | SGYKQPVSLGVDMGTRHVGISATTKKDVLFEAEAQLRTDIVELL | |
| by | | AIRRQFRRSRRNRKTRYREARFLNRRKPEGWLPPSIQHKINSHIK | |
| ISGst5) | | LIDLVHNILPVTSVTIEVAAFDTQKLKNLNIRGVEYQQGEQMGF | |
| | | WNVREYVLYRDNHTCQYCKGKKKDPVLQVHHIESRKTGGDSP | |
| | | DNLITLCKTCHHEIHEKGLEHIFQRKRPPMRDASQMTAMRWAM | |
| | | FSRIREKYSHANITYGYITKCIRIANGLSKSHMVDARCISGNPLAK | |
| | | PSGTVYLLKFVRKNNRQLHKATILKGGKRKSNKAPRFVKGFQLF | |
| | | DKVVYERKECFIFGRRSSGYFDLRLLDGTKVHASASWKKLKRV | |
| | | EHASTLLIERRKGDSSPTFALA | |

TABLE 2

| | | Guide RNA sequence, 5′→3′ |
|---|---|---|
| | | (scaffold sequence as shown; |
| Nuclease | Protein amino acid sequence | guide region shown as N's) |
| GstTnpB1 | MVANKAYQFRLYPTKEQEQLLAKTFGCVRFV | GGAUGUCACAAGCCCUCCAUUUCGGUCAAGG |
| (ISGst4) | YNKMLEERIQLFEKFKDDQESLKQQTCPTPAK | CGUUCAUGACAACGGCUGGGGCAUGUUCACC |
| | YKKEFPWLKEVDSLALANAQLNLQKAFQHFF | ACUUUCCUUCAGUACAAGCUGGCCGAACAGG |
| | SGRAGFPKFKNRKAKQSYTTNMVNGNIKLSD | GGAAGAAGCUGAUCAAAAUCGACAAAUGGUU |
| | GYIKLPKLKWIKFKQHREIPAHHIIKSCTITKT | CCCCUCGUCCAAAACGUGUUCGUGCUGCGGU |
| | KTGKYYISILTEYEHQPALKEVQTVVGLDFSM | CGAGUCAAGGAGUCUCUAUCGCUUUCUGAAC |
| | STLYVDSEGKRANYPRFYRKALETLAKEQRK | GGACAUUCCGCUGUGAAUGUGGAUUCGAGAG |
| | LSRKKKGSNRWHKQRLKVAKLHEKMANQR | CGACAGGGACGUCAAUGCGGCCAUCAAUAUC |
| | KDFLHKESHKLAKRYDCVVIEDLNMKGMSQ | AAACAUGAGGGCAUGAAACGAUUGGCGAUCG |
| | ALHFGQGVHDNGWGMFTTFLQYKLAEQGKK | UCUAAUUUGUCCUCGAACCGUGGGGCACACG |
| | LIKIDKWFPSSKTCSCCGRVKESLSLSERTFRC | GGGAUCGCUCGGUCAACUUCCCAUCAUGAGA |
| | ECGFESDRDVNAAINIKHEGMKRLAIV (SEQ | UGGGAUUACCCGAGAAGCCCCCACCUCUAAG |
| | ID NO: 6) | CGAAGCGUAGGUGGUGGGAGUAUGUCACNNN |
| | | NNNNNNNNNNNNNNNNNNNNNNNNNNNNNN |
| | | NNNNNNN (SEQ ID NO: 12) |
| GstTnpB3 | MGYFCIKQQLNGLTKEEYLTLRELCHIAKNM | UUAAGCGCGGCUUGUAUCGAACAAAGUCUGG |
| (ISGst1) | YNVGLYNVRQYYFEHKEFLNYEKNYHLAKT | CAAACUAAUUAAUGCUGAUGUCAAUGGCGCA |
| | NENYKLLNSNMAQQILKKVNEAFKSFFGLISL | UUAAACAUCUUAAAGAAAAGUAAAGCUGUAG |
| | AKQGKYDHKAISIPKYLKKDGFHSLIIGQIRID | ACCUGAGUGUCUUAUGCUCUAGCGGCGAAGU |
| | GNKFTIPYSRLFKKTHKPITITIPPVLLDKKIKQI | GGACACGCCUCAAAGAAUAAGGAUUGCUUGA |
| | EIIPKHHARFFEIQYKYEMPEDQRELNDQKAL | AGCAGUCAAACUUCUUUGGAAGCCCCCACUU |
| | AIDLGLNNLATCVTSDGRSFIIDGRRLKSINQW | CAAAUUUUCGCUAGAAAAUUAAGUGGGGGUA |
| | FNKENARLQSIKDKQKIKGTTRKQALLAMNR | GUUCACNNNNNNNNNNNNNNNNNNNNNNNNN |
| | NNKVNDYINKTCRYIINYCIENQIGKLVIGYAE | NNNNNNNNNNNNNNN (SEQ ID NO: 13) |
| | TWQRNMNLGKKTNQNFVNIPLGNIKEKLEYL | |
| | CEFYGIEFLKQEESYTSQASFFDGDEIPEYNAD | |
| | NPKEYKFSGKRIKRGLYRTKSGKLINADVNG | |
| | ALNILKKSKAVDLSVLCSSGEVDTPQRIRIA | |
| | (SEQ ID NO: 7) | |
| GstTnpB4 | MGYRTLKTRFRAKKEVIQKLFECNRISAEVW | UUUACGGUCAAACCGUUUACGUAUCGACGGA |
| (ISGst3) | NECLRLAKEHHLETGKWITKTELQKATKGRF | UUGCUUAGGUAUGAAGUCGUAGAUGGCGAGU |
| | SIHSQSIQAVVHKYIFARDGAKEARKKGEKIK | UCCCGCCCUUGAGUAUCAUGGAUACUCAUGU |
| | YPYKKKKHFNTKWAKDGFVLHEDGTLELSL | UGCCUGUGGCUCCCAACGGGACGGGUGCCAG |
| | GIWNRKRQSPLVVKIDKEKLPNGKVKEIELVY | GUGUUGCCUGCAGAUAGGCUGCCAACCAGCC |
| | DRGLWLCLSYEDGKKPKENPCKNRVAIDPGE | UAUCAUGCAUCCACCUCCGAAGGGGAAACAG |
| | IHTIAAICENGESLIITGRKIRSIHRLRNKKLKEL | GAAACCCCCACUUCGAUAAGUGGGGGAGGUU |
| | QKLMSRCKKGSKQWKKYNRAKQYVLSKSEA | CAUNNNNNNNNNNNNNNNNNNNNNNNNNNN |
| | QLKDALHKTTRQFVRWCLENQVKEVVIGDIE | NNNNNNNNNNNNN (SEQ ID NO: 14) |
| | GIQRNTKKKRNKKTNQKLSNWSFGKLFDYLK | |
| | YKLNAEGIQIEKKDESYTSQTCPVCGKKNKSS | |
| | SRNYTCQCGYKRHRDIHGAMNLFAKVYYGEI | |
| | RPLEFTVKPFTYRRIA (SEQ ID NO: 8) | |
| GstTnpB5 | MDMTLTAKIKIYPTAEQAEVLKATLSAYRQA | UAUACCUCAAACGAUGACCGCAUUGGUGCUA |
| (ISGst5) | CNAVSVVIFDTKVLAQAKLHDMTYRLLRSNY | UGAACCUUCAACGAAAAGGAAUAGAGUACAU |
| | ALRSQMAQSVIKTVIARYRSLKSNGHEWTLV | CGUUGAAGGAACGACACAGGCAUGACCUGCG |
| | RFKKPEYDLVWNRDYSIVQGLFSVNTLEGRIK | UCGUUGGGUAGUUGUCAACCUGCCCCCUGAU |
| | VSFEPKGMEPYFDGSWTFGTAKLVYKHNKFF | GCAACCCCAAGUCAAGGAAGGAGGACGAAGU |
| | LHIPMTKTIPTVDEHNIRQVVGVDVGVNFLAV | CCGCUCGCACUUCUGGGGAGUUGCAAGCCCC |
| | AYDSQGKTIFFNGRKIKHMRAKYKRMRKTLQ | CACUUCAAGCGUUAGCUAAGUGGGGGUAGUU |
| | QKGTASARRKLKTIGQRENRWMTDVNHAVT | GACNNNNNNNNNNNNNNNNN |
| | KALVRQYGERTLFVLEDLTGIREKTERVRIHD | (SEQ ID NO: 15) |
| | RYETVSWAFYQFRQMLEYKARLHGSKVIVV | |
| | APHYTSLTCPKCGHTEKANRKKRTHTFCCRT | |
| | CGYTSNDDRIGAMNLQRKGIEYIVEGTTQA | |
| | (SEQ ID NO: 9) | |
| GstIscB1 | MGFVYIINKHGKPLMPCSPRKARLLLKQKKA | NNNNNNNNNNNNNNNNNNNNNNNNNNNNNN |
| (ISGst2) | KVVRRTPFTIQLLYGSSGYKQPVSLGVDMGT | NNNNNNNNNNGUCAACUCCCCACCACUUAAA |
| | RHVGISATTKKDVLFEAEAQLRTDIVELLAIR | GCCUAACGGCUUUUGAAGUGGGGGCUUGGCA |
| | RQFRRSRRNRKTRYREARFLNRRKPEGWLPP | AGCCCUAGUUGACUACCCUCAGCCAUUCAAU |
| | SIQHKINSHIKLIDLVHNILPVTSVTIEVAAFDT | GGCUACGUUGGAAGGGUCAUGACACUUACGG |
| | QKLKNLNIRGVEYQQGEQMGFWNVREYVLY | AUGCUCCUCCAGUCCGUAACCCUGUCGCAGA |
| | RDNHTCQYCKGKKKDPVLQVHHIESRKTGG | UGGUUAAAAGCUCUGAUGGGUAGGAGCGGUG |
| | DSPDNLITLCKTCHHEIHEKGLEHIFQRKRPPM | CUGUCUGCCGAACAAGCCCUUCCAACAUUGG |
| | RDASQMTAMRWAMFSRIREKYSHANITYGYI | GGAGGAGGAACUCACUCCUGAAAGGAGGUAC |
| | TKCIRIANGLSKSHMVDARCISGNPLAKPSGT | ACGUCUUGUUCGUGUACAUUAUCAAUAAGCA |
| | VYLLKFVRKNNRQLHKATILKGGKRKSNKAP | UGGAAAACCACUC (SEQ ID NO: 16) |
| | RFVKGFQLFDKVVYERKECFIFGRRSSGYFDL | |
| | RLLDGTKVHASASWKKLKRVEHASTLLIERR | |
| | KGDSSPTFALA (SEQ ID NO: 10) | |
| GstTnpA | MKLDNNNHSVFLLYYHLVLVVKYRRQVIDD | |
| (IsGst4) | TISDYAKDMFVRLGKNYNISLVEWNHDMDH | |
| or | VHILFKAHPNSELSKFINAYKSASSRLIKKHFP | |
| GstTnpA | QVKRKLWKEYFWSRSFCLLTTGGAPIEVIKK | |
| (encoded | YIENQGMK (SEQ ID NO: 11) | |
| by | | |
| ISGst1) | | |

TABLE 3

| | | SEQ ID |
|---|---|---|
| Description | Sequence | NO: |
| CboTnpB | MGIVAIKIKLKPTKEQEVLFWKSAGVARWSYNYFLSESERHYQEYLEGKQDKKTIKESEV | 17 |
| | RKYINNVLKKTTHTWLEEVGSNVMKQAVKDADIARKRWFDGVANKPKFKSKRKSKVSF | |
| | YVNYESLKRTNNGFRGEKIGVVKTYQALPKLKKGEKYSNPRISFDGMNWFISIGYNNEFK | |
| | AVKLTDVSLGIDVGIKELAVCSDGQFKKNINKTKRVRFLEKKLKREQRKISHKLEANIKSY | |
| | DKNRKPIYKRPLRDMKNIQKQNRIIRNLYKKLENIRTNYLHQCSNEIVKTKPSRIVMETLNI | |
| | KGMMKNKHLSKAIANQKLYEFKRQIQYKCKKYGIKFVEADKWYPSSKTCSCCGQVKSDL | |
| | KLKDRLYVCNCGLKMDRDLNASINLANYQIQSA | |
| CboTnpB | MGIVAIKIKLKPTKEQEVLFWKSAGVARWSYNYFLSESERHYQEYLEGKQDKKTIKESEV | 18 |
| (D190A) | RKYINNVLKKTTHTWLEEVGSNVMKQAVKDADIARKRWFDGVANKPKFKSKRKSKVSF | |
| | YVNYESLKRTNNGFRGEKIGVVKTYQALPKLKKGEKYSNPRISFDGMNWFISIGYNNEFK | |
| | AVKLTDVSLGIAVGIKELAVCSDGQFKKNINKTKRVRFLEKKLKREQRKISHKLEANIKSY | |
| | DKNRKPIYKRPLRDMKNIQKQNRIIRNLYKKLENIRTNYLHQCSNEIVKTKPSRIVMETLNI | |
| | KGMMKNKHLSKAIANQKLYEFKRQIQYKCKKYGIKFVEADKWYPSSKTCSCCGQVKSDL | |
| | KLKDRLYVCNCGLKMDRDLNASINLANYQIQSA | |
| ωRNA | aauuaccaaauucaaagugcuuaaccuaaacuaagcuuugaauauguacguaacgauacuacg | 19 |
| (native | gaauuuaagccuguuaagagucauaucaaaugguaguagcuuuggcaaacccagauucguug | |
| sequence | aagcaggaagacacaagacgaaaaucauaagacauaagucgugucauaguauaaaugucuaca | |
| guide) | uuuaaauagaaauguauauauuuuucguaucgguugugaauuuuuagccaauaaauuuaaugg | |
| | ugaaaaagg | |
| ωRNA | aauuaccaaauucaaagugcuuaaccuaaacuaagcuuugaauauguacguaacgauacuac | 20 |
| (library | ggaauuuaagccuguuaagagucauaucaaaugguaguagcuuuggcaaacccagauucguug | |
| targeting | aagcaggaagacacaagacgaaaaucauaagacauaagucgugucauaguauaaaugucuaca | |
| guide) | uuuaaauagaaauguauauauuuuucguaucgggacaagugguucaccaugcguugcuuuaug | |
| | guaugauag | |
| CboTnpA | MELMSIGKFAKRVGVNVVTLRRMEAKGEFLPAHVSSGGTRYYSTDQLKYFGKERNAHKL | 21 |
| | VVGYCRVSTPSQKDDLENQVNNVKSYMIAKGYQFEIIKDIGSGINYKKKGLKELIDKINNQ | |
| | EVSKVVILYKDRLIRFGFELIEYLCQINNVELEIIDHSEKSKEEELTDDLIQIITVFANRLYGQ | |
| | RSKKTKRLIEEVKNNDSSN | |
| CboTnpB | MIVAIKIKLKPTKEQEVLFWKSAGVARWSYNYFLSESERHYQEYLEGKQDKKTIKESEVR | 22 |
| | KYINNVLKKTTHTWLEEVGSNVMKQAVKDADIARKRWFDGVANKPKFKSKRKSKVSFY | |
| | VNYESLKRTNNGFRGEKIGVVKTYQALPKLKKGEKYSNPRISFDGMNWFISIGYNNEFKA | |
| | VKLTDVSLGIDVGIKELAVCSDGQFKKNINKTKRVRFLEKKLKREQRKISHKLEANIKSYD | |
| | KNRKPIYKRPLRDMKNIQKQNRIIRNLYKKLENIRTNYLHQCSNEIVKTKPSRIVMETLNIK | |
| | GMMKNKHLSKAIANQKLYEFKRQIQYKCKKYGIKFVEADKWYPSSKTCSCCGQVKSDLK | |
| | LKDRLYVCNCGLKMDRDLNASINLANYQIQSA | |
| CseTnpB1 | MGTIIKNVKQYSYILDTEMLNELKFIANQYKNVKNYIYSRYSGINSIPLLSKERQIRNEWVK | 23 |
| | SEFAKQWKLPARYWKLALTEAMGNIKSKWFNIKSKIKEQVKVNENLSDEDKHYINYIVKF | |
| | NSYYHKVLNYIGFDIPKIFKKKNLNFKYLNNLIRRYTRKYKGHIPYSKIGKTFSIDTGLYSY | |
| | KNNCINITSTKKGKRLSIKLTDSNKYNRTMIVKIIDNKIVVNCPLKIKTRKNKNTNIIGIDKG | |
| | YRYLFAVSSDKFYGENLNNYLSKETERLNKVNAQRNRFWALYNQYLDKGYIKKANKIKE | |
| | NNLGKVKYNHNKLKHDQTVKSYINYSLNQLIKEEKPKEIVMENLDFVNWNDKYPKIVKC | |
| | KLSRWIKGYIRERVEYKCDFYSIKYTYINPAYTSKVCSVCGSFGKRDGDIFICPKCNKFHAD | |
| | INASKNILNRKYDNDITLYTNYKKVKDILEKRIKVS | |
| CseTnpB2 | MIIATKIKLKPTNEQEILFWKSAGVARWSYNYFLAESENYYSQYNKTLKEGEVRKHINNV | 24 |
| | LKKTTHTWLSEVGSNVMKQAVKDANLARKRWFEGLSSKPKFKSRRKSKISFYVNYESLK | |
| | VVNGGFRGEKIGFIKTYQPLPKLKKGEKYSNPRISFDGRSWFLSVGYEKEFEAIELTGKSLG | |
| | IDVGIKELAVCSDGEFKKNINKTKKVKNLKRKLRREQRKVSRKIEANIKSYDKNRRPIYKT | |
| | PLRDMKNIQKQNQIIRNLYKKLTDIRTNHLHQCTSEIVKTKPSRIVMETLNIKGMMKNKHL | |
| | SKAIQEQNLYEFKRQIKYKCEIYGIEFVEADKWYPSSKTCSCCGAIKKDLKLKDRTYVCPC | |
| | GLKLDRDLNASINLANYSIQSA | |
| CseTnpA1 | MELMSIGKFAKKVGVNVVTLRRMEKKGELLPAHVSSGGTRYYSIEQLSYFGKATNTNKL | 25 |
| | VIGYCRVSTPSQKDDLVNQVNNVKSYMIAKGYQFEIITDIGSGINYKKKGLKQLINKINNR | |
| | EISKVVILYKDRLVRFGYELIEYMCEINGIELEIIDHSEKSKEEELTDDLIQIITVFANRLYGQ | |
| | RSKKTKKLIDEVKNNDNSN | |

TABLE 4
wRNA sequences

| | | Protein | | |
|---|---|---|---|---|
| | | SEQ ID | Target | Guide |
| Protein | wRNA sequence | NO | sequence | origin |
| CboTnpB | UAUGUACGUAACGAUACUACGGAAUUUAAGCCUGUUA | 22 | TGGGNNNN | Universal |
| | AGAGUCAUAUCAAAUGGUAGUAGCUUUGGCAAACCCA | | NNNNNNNN | guide |
| | GAUUCGUUGAAGCAGGAAGACACAAGACGAAAAUCAU | | NNNNNN | |
| | AAGACAUAAGUCGUGUCAUAGUAUAAAUGUCUACAUU | | | |
| | UAAAUAGAAAUGUAUAUAUUUUUCGUAUCGGNNNNNN | | | |
| | NNNNNNNNNNNN (SEQ ID NO: 26) | | | |
| CboTnpB | UAUGUACGUAACGAUACUACGGAAUUUAAGCCUGUUA | 22 | TGGGTTGTG | Native |
| | AGAGUCAUAUCAAAUGGUAGUAGCUUUGGCAAACCCA | | AATTTTTAG | guide |
| | GAUUCGUUGAAGCAGGAAGACACAAGACGAAAAUCAU | | CCAA (SEQ | |
| | AAGACAUAAGUCGUGUCAUAGUAUAAAUGUCUACAUU | | ID NO: 202) | |
| | UAAAUAGAAAUGUAUAUAUUUUUCGUAUCGGUUGUGA | | | |
| | AUUUUUAGCCAA (SEQ ID NO: 27) | | | |
| CseTnpB2 | CUAGGUUCUGAAUAUGUACGUAACGAUACUACGGAAU | 24 | TGGGNNNN | Universal |
| | UCAAGCCUGUGGAGGGUUAUAACAAAUGAAAGUAGUA | | NNNNNNNN | guide |
| | UGUGAUUUAUCACUACAAAAUCAGAUUCGUUGAAACA | | NNNNNN | |
| | GGAAGAUACAAAAUGAAAGCCUUAUGGUAUAAGUUGU | | | |
| | AUCACAGUAGAAAUGUCAACAUUAUUAUAAUAAUGUA | | | |
| | UAUAUUUUUCGUAUCGGNNNNNNNNNNNNNNNNNN | | | |
| | (SEQ ID NO: 28) | | | |
| CseTnpB2 | CUAGGUUCUGAAUAUGUACGUAACGAUACUACGGAAU | 24 | TGGGATTCA | Native |
| | UCAAGCCUGUGGAGGGUUAUAACAAAUGAAAGUAGUA | | TGATTTCAT | guide |
| | UGUGAUUUAUCACUACAAAAUCAGAUUCGUUGAAACA | | TAGG (SEQ | |
| | GGAAGAUACAAAAUGAAAGCCUUAUGGUAUAAGUUGU | | ID NO: 203) | |
| | AUCACAGUAGAAAUGUCAACAUUAUUAUAAUAAUGUA | | | |
| | UAUAUUUUUCGUAUCGGAUUCAUGAUUUCAUUAGG | | | |
| | (SEQ ID NO: 29) | | | |

TABLE 5
Protein Sequences and wRNA sequences

| | | Protein | | wRNA |
|---|---|---|---|---|
| | | SEQ ID | | SEQ ID |
| Protein | Protein amino acid sequence | NO | wRNA sequence | NO |
| CseTnpB3 | VILAKKVRLYPTKEQEQKLWQSVGTARFIYNW | 30 | AGAUAAUGUUAAUAUG | 51 |
| | TLAKQQENYKNGGKFISDNVLRKEITQLKKTEL | | UAGGAUUCGUUGUAUC | |
| | NWLNEVSNNVAKQAVKDGCNAYKRFFNGLSD | | CGAAUUUAAGCCUUUG | |
| | KPRFKSRRKCKPSFYNDTSKLKIKDNVVLIEKV | | GACUGUCAUAUCAAAU | |
| | GWITIRKNSIPMNCKYTNPRISFDGKYWFIAVGI | | GAGAGUAGCAAAUGAA | |
| | EKEKLLVELTDESIGIDVGVKDLAICSNRMTFKN | | UUUAUUCAUUGCAAAA | |
| | INKTKEVKRLKKVLKRKQRKISRKYEINKIESEV | | UCAGACAGGGUGAAUA | |
| | KSRCQFKKTNNIIKLEKEIRLLHRRLANIRSNHIH | | AGGAAUUAAACAAGAU | |
| | QATNMIVRTKPSRVVMETLNIKNMMKNRHLSK | | UUAUAGAUUUUUAUAA | |
| | AIAEQSLYDFKVKMKYKCAIYGIEFVEADKWFP | | AUUUUUGGCAACGGUG | |
| | SSKTCSKCGAIKKDLKLKDRVYQCSCGLKIDRD | | AUAGGGAUGUUUAGUG | |
| | FNASINLSRYKLA | | (SEQ ID NO: 51) | |
| CseTnpB4 | VILAKKVRLYPTKEQEQKLWQSVGTARFIYNW | 31 | GAGGUGUACCAUGCGU | 52 |
| | TLAKQQENYKNGGKFISDNVLRKELTILKKTEL | | UGUAUGGGAAGUUAAG | |
| | NWLNEVSNNVAKQAVKDGCNAYKSFFKGLSD | | CCCUUGGAGUGUUAUA | |
| | KPRFKSRRKSKPSFYNDTLKLKIKDNVVLIEKV | | UCAAACCAAAGUAGCU | |
| | GWITIRKNSIPMNCKYTNPRISFDGKYWFIAVGI | | CUUAGCAAAAUGGGAC | |
| | EKEKLLVELTDESIGIDVGVKDLAICSNGMSFKN | | ACGUUGAAUAGGGAAU | |
| | INKTKVIKKLKKTLKRKQRQCSRKYEKNKKGR | | UAAACAAGCUUUAUAA | |
| | EFVKTKNIAKLERQIRLLHRRLANIRDNHLHQA | | AGUUUUAUAAUGUUUU | |
| | TNKIVKTKPSRVVMETLNIKGIMKNKHLSKAIA | | GGCAACGGGAUAAGAA | |
| | EQCLYEFKRQMQYKCELYGIEFVEADKWYPSS | | UAACAUGAGA | |
| | KTCSECGHVKTKLSLSERTYICEECGCVIDRDY | | (SEQ ID NO: 52) | |
| | NASINLSRYSA | | | |
| CseTnpB5 | LKRAYKIEIKPTLAQKIKIHQTIGISRFIYNFYLAH | 32 | UAGAUACUUAUAUAUG | 53 |
| | NKEIYQKEKRFVSGMDFSKWLNNEYIPKNQDE | | UACCGUUGGCUAGAUG | |
| | KWIKEVSSKATKQAIMNGEKAFKKFFKGESGFP | | GGAAUUUAAGCCUGUG | |
| | KFKKKKNKDVKAYFPKNNKTDWTIERHRVKIP | | GAGUGCUAUAUAAACU | |
| | TIGWMRLKEYGYIPTNSIVKSGTVSQKADRYYV | | AAAGUAGCUUCGGCAA | |
| | SILVEEDIKPNDKPYSCGIGVDLGIKDTAICSNGI | | AAUGGAGUACAUUGAA | |
| | KFKNINKTSKVKKIETKLKREQRKLSRKYESLKI | | ACAGGAAAAAUCUCAA | |
| | RNKNNKEGATRQNIQKQVVKVQKIHQRLYNIR | | UAUGGGUAUUUUUGUC | |
| | NDYNNKIVSELVKIKPVFITIEDLAISNMIKNKHL | | CAUAUUUUGAGUAGCA | |
| | SKAVQKQKLYDLRNKLISKSHQNNIEIRQVSRW | | GAUGUAUUACCAAGUG | |
| | YPSSKLCNKCGGVKKDLKLSDRVYNCKCGYSF | | AAAU | |
| | DRDANASYNLRDAKEYKII | | (SEQ ID NO: 53) | |
| CseTnpB6 | LLRAYKIEIKPTLEQKIKIHQTIGISRFIYNFYLAH | 33 | AGAUACUUAUAUAUGU | 54 |
| | NKEIYQKEKRFVSGMDFSKWLNNEYIPNNQDK | | ACCGUUGGCUAAAUGG | |
| | KWIKEVSSKATKQAIMNGEKAFKKFFKGETSFP | | GAAUUUAAGCCUGUGG | |
| | KFKKKKNKDVKAYFPKNNKTDWTIERHRVKIP | | AGUGCUAUAUAAACCA | |
| | TIGWMRLKEYGYIPNNSIVKSGTVSQKSDRYYV | | AAGUAGCUUCGGCAAA | |
| | SILVEEDIKPNYKPYSCGIGIDLGIKDTAICSNGIK | | AUGGAGUACAUUGAAA | |
| | FKNINKMSKVKKIERKLKREQRKLSRKYESLKI | | CAGGAAAAAUCUCAAU | |
| | RNKNNKEGATRQNIQKQVVKVQKIHQRLYNIR | | AUGGGUAUUUUUGUCC | |
| | NDYNNKIVSELVKIKPVFITIEDLAISNMIKNKHL | | AUAUUUUGAGUAGCAG | |
| | SKAVQKQKLYDLRNKLISKSHQNNIEIRQVSRW | | UGUUGAAAAUGAAAAA | |
| | YPSSKLCNKCGSVKKDLKLSDRVYNCKCGYSF | | AGU | |
| | DRDANASYNLRDAKEYKII | | (SEQ ID NO: 54) | |
| CseTnpB7 | LKRAYKIEIKPTLAQKIKIHQTIGISRFIYNFYLAH | 34 | UAGAUACUUAUAUAUG | 55 |
| | NKEIYQKEKRFVSGMDFSKWLNNEYIPNNQDK | | UACCGUUGGCUAGAUG | |
| | KWIKEVSSKATKQAIMNGEKAFKKFFKGETSFP | | GGAAUUUAGGCCUGUG | |
| | KFKKKKNKDVKAYFPKNNKTDWTIERHRVKIP | | GAGUGCUAUAUAAACC | |
| | TIGWMRLKEYGYIPTNSIVKSGTVSQKSDRYYV | | AAAGUAGCUUCGGAAA | |
| | SILVEEDIKPNDKPYSCGIGIDLGIKDTAICSNGIK | | AAUGGAGUACAUUGAA | |
| | FKNINKTSKVKKIERKLKREQRKLSRRYESLKIR | | ACAGGAAAAAUCUCAA | |
| | NKNNKEGATRQNIQKQVVKVQKIHQRLYNIRN | | UAUGGGUAUUUUUGUC | |
| | DYNNKIVSELVKIKPVFITIEDLAISNMIKNKHLS | | CAUAUUUUGAGUAGCA | |
| | KAVQKQKLYDLRNKLISKSHQNNIEIRQVSRWY | | GUAAGUUUAAUGAAGA | |
| | PSSKLCNKCGSVKKDLKLSDRVYNCKCGYSFD | | AGAAA | |
| | RDANASYNLRDAKEYKII | | (SEQ ID NO: 55) | |
| CseTnpB8 | VILAKKVRLYPTKEQEQKLWQSVGTARFIYNW | 35 | AGAUAAUGUUAAUAUG | 56 |
| | TLVKQQENYKNGGKFISDNVLRKELTILKKTEL | | UAGGAUUCGUUGUAUC | |
| | NWLNEVSNNVAKQAVKDGCNAYKNFFKGLSD | | CGAAUUUAAGCCUUUG | |
| | KPRFKSRRKSKPSFYNDTSKLKIKDNVVLIEKVG | | GACUGUCAUAUCAAAU | |
| | WITIRKNSIPVNCKYTNPRISFDGKYWFIAVGIE | | GAGAGUAGCAAAUGAA | |
| | KEKLLVELTDESIGIDVGVKDLAICSNGMTFKNI | | UUUAUUCAUUGCAAAA | |
| | NKTKEVKRLKKVLKRKQRKISRKYEINKIESEV | | UCAGACAGGGUGAAUA | |
| | KSRCQFKKTNNIIKLEKEIRLLHRRLANIRSNHIH | | AGGAAUUAAACAAGAU | |
| | QATNMIVRTKPSRVVMETLNIKNMMKNRHLSK | | UUAUAUAUUUUUAUAG | |
| | AIAEQSLYDFKVKMKYKCAIYGIEFVEADKWFP | | AUUUUUGGCAACGGUG | |
| | SSKTCSKCGAIKKDLKLKDRVYQCSCGLKIDRD | | UUAAUAUUGUUUGAUU | |
| | FNASINLSRYKLA | | (SEQ ID NO: 56) | |
| CseTnpB9 | MKLSFKFRPNVSSKQLEIIEELSYHTTKLYNIINY | 36 | — | |
| | ALRENGFKNYYNIELEFKNNWHCDFLHSHTRQ | | | |
| | QMFKVLEQNWKSYFASIKDYEVNPSKYKGIPRR | | | |
| | PKFKNVDKNKNEIIFTNIAIRFQDGILKLSLSKAI | | | |
| | QRLFEVESLNFEVSNKLQSLIDWNSLQQARLTY | | | |
| | DKVQKCWCLIVIYNKLEKENNNSNIMAIDLGLD | | | |
| | NLATLTFKEGNETYIFCGKKLKSVNAYANKKIA | | | |
| | YLQSIEMQKCGSDKFKNTKEINRLRRYRNNYID | | | |
| | DYLHKVSKNIIDKAIEHEVKKIVIGKLKGIKQDM | | | |
| | NYNKSFVQIPIQRLAELIKYKAKLQGIEVKFKEE | | | |
| | SYTSGCSAFDLEPINKKYYDKTRRVVRGLFKSS | | | |
| | FGLVNSDINGSLNILRKEEKCIPQLVKTMRDKG | | | |
| | KCSRPLRIRVAC | | | |
| CseTnpB10 | LKRAYKMEIKPTLAQKIKIHQTIGISRFIYNFYLA | 37 | UAGAUACUUAUAUAUG | 57 |
| | HNKEIYQKEKRFVSGMDFSKWLNNEYIPNNQD | | UACCGUUGGCUAAAUG | |
| | KKWIKEVSSKATKQAIMNGEKAFKKFFKGEAG | | GGAAUUUAAGCCUGUG | |
| | FPKFKKKKNKDVKAYFPKNNKTDWTIERHRVK | | GAGUGCUAUAUAAACC | |
| | IPTIGWMRLKEYGYIPTNSIVKSGTVSQKADRY | | AAAGUAGCUUCGGCAA | |
| | YVSILVEEDIKSNDKPYSCGIGVDLGIKDTAICSN | | AAUGGAGUACAUUGAA | |
| | GIKFKNVNKTSKVKKIERKLKREQRKLSRKYES | | ACAGGAAAAAUCUCAA | |
| | FKIRNKNNKEGATRQNIQKQVVKVQKIHQRLY | | UAUGGGUAUUUUUGUC | |
| | NIRNDYNNKIVSELVKIKPVFITIEDLAISNMIKN | | CAUAUUUUGAGUAGCA | |
| | KHLSKAIQKQKLYDLKNKLISKSHQNNIEIRQVS | | GGAAAAAAUUAUGUAU | |
| | RWYPSSKLCNKCGSIKKDLKLSDRVYNCKCGY | | GAUGU | |
| | RFDRDANASYNLRDAKEYKII | | (SEQ ID NO: 57) | |
| CseTnpA2 | MKYYSIGEFATQIGKTIQTLRNWDKNGTLKPSH | 38 | — | |
| | ITAGGTRYYSQEQLNHFLGLKSEVQLSKKTIGY | | | |
| | CRVSSHKQKDDLARQIENVKTYMYAKGYQFEII | | | |
| | QDIGSGINYNKKGLNQLIDMITNSEVDKIVVLY | | | |
| | KDRLIRFGYELIENLCNKYGTTIEVIDNTEKSEE | | | |
| | QELVEDLIQIVTVFSCRLQGKRADKAKKMIKEL | | | |
| | LESDSSQES | | | |
| CseTnpA3 | VKYYSIGKFSKLIGRTTQTLRDWDKKGVLKPQH | 39 | — | |
| | VAPSGYRYYSQEQLNHFLGIKGIETKKVIGYCR | | | |
| | VSSHKQKDDLARQIENVKTYMYAKGYQFEIIQD | | | |
| | IGSGINYSKKGLNQLIDMITNSEVEKIVILYKDRL | | | |
| | LRFGFEIIENLCNKYGTTIEIIDNTEKTEEQELVE | | | |
| | DLIQIVTVFSCTLQGKRANKVNKMIKELLESDPS | | | |
| | QES | | | |
| CseTnpA4 | MKHITNYKPKDFAELLYVSVKTLQRWDREDIL | 40 | — | |
| | KAKRTPTNRRYYTYDQYLEFKGLKKEIERKIIIY | | | |
| | TRVSTNNQKDDLKNQIEFLKNFANVKGIIVDDV | | | |
| | ISDIGSGLNYNRKKWNKLLDECMENKIDSILITH | | | |
| | KDRFIRFGYNWFERFLVKFDVKIIVVNNKSFSPQ | | | |
| | EESVQDKISILHVFSCRIYGLRKYKKKIKEDEEIE | | | |
| | KSLQDRD | | | |
| CseTnpA5 | MKYYSIGEFATRIGKTIQTLRNWDKNGTLKPSHI | 41 | — | |
| | TAGGTRYYSQEQLNHFLGLKSEVQLSKKTIGYC | | | |
| | RVSSHKQKDDLPKQIENVKTYMYAKGYQFEIIQ | | | |
| | DIGSGINYNKKGLNQLIDMITNSEVDKIVVLYK | | | |
| | DRLIRFGYELIENLCNKYGTTIEVIDNTEKSEEQE | | | |
| | LVEDLIQIVNVFSCRLQGKRADKAKKMIKELLE | | | |
| | SDSSQES | | | |
| CdiTnpB1 | MIKTHRVKLNLTRTQFELVREKQMESANCWNY | 42 | — | |
| | IVNLSKEYYFEHKQWIKKNDIQKLIKGKYNLHS | | | |
| | QTIQAISDKFDANRKTISELRKKGNTKAKYPYK | | | |
| | TKKFYIIPFKASAINRNAKGNLKLSMSKGRYLEL | | | |
| | DFNVENIKTAEIVWRNGYYLYYTFDNELNGVV | | | |
| | AKGTNTVGVDLGEIHSIASVTNEGVGLILSNREG | | | |
| | RSIKQFRNKMYAYISKRLSKCKKGSRQSKKLW | | | |
| | RLKNKIRSKTDNQLMNLYHQTTRKFIDFCVEQK | | | |
| | VCEIVLGDIKGVEKDTKKKKRLNRVNRQKISQ | | | |
| | MEYGRIKDYIKYKAKEQGIEVKLVKENYTSQTC | | | |
| | PKCSKKHKPTGRTYSCTCGYETHRDIVGAWNIL | | | |
| | NKKHKYGLVDFRINHKQPINLKVSTV | | | |
| CdiTnpB2 | VIAVEKAYKFRMYPNKKQQELINKTFGCCRFV | 43 | — | |
| | YNKYLAKRIDVYKNDKETFTYKQCSSDLTNLK | | | |
| | KELKWLKEPDKFSLQNALKDLDNAYKKFFKEK | | | |
| | AGLPKFKSKKINRFSYKTNFTNGNIMYCGQHIK | | | |
| | LPKLGMVKIRDKQVPQGRILNATISKEPSGRYY | | | |
| | VSLCCTDVDIEVFENTNNQIGLDLGIKEFCISSCG | | | |
| | EFIENPKYLKKSLNKLAKLQRELSRKTIGSLNRN | | | |
| | KARLKVAGLQEHIANQRKDFLQKLSTKLIKEND | | | |
| | IICIEDLQVKNMIRNRKLSRLISDVSWSEFIRQLE | | | |
| | YKANWHGRQIVKVGKFFASSQICNKCGYKNEE | | | |
| | VKDLNIREWICPSCNETHDRDINASINILKEGLR | | | |
| | LITIQNK | | | |
| CdiTnpB3 | VIAVKKAYKFRMYPNKKQQELINKTFGCCRFV | 44 | — | |
| | YNKYLAKRIDVYKSDKETFTYKQCSSDLTNLK | | | |
| | KELKWLKEPDKFSLQNALKDLDNAYKKFFKEK | | | |
| | AGFPKFKSKKINRFSYKTNFTNGNIMYCGQYIK | | | |
| | LPKLGMVKIRDKQVPQGRILNATISKEPSGRYY | | | |
| | VSLCCTDVDIEVFENTNNHIGLDLGIKEFCISSCG | | | |
| | DFIENPKYLKKSLNKLAKLQRELSRKTIGSLNRN | | | |
| | KARLKVAGLQEHIANQRNDFLQKLSTKLIKEND | | | |
| | IICIEDLQVKNMIRNRKLSRLISDVSWSEFIRQLE | | | |
| | YKANWYGRQIVKVGKFFASSQICNKCGYKNEEI | | | |
| | KDLNIREWICPSCNETHDRDINASINILKEGLRLI | | | |
| | TIQNK | | | |
| CdiTnpB4 | VIAVKKAYKFRIYPNKKQQELINKTFGCCRFVY | 45 | — | |
| | NKYLAKRIDVYKNDKETFTYKQCSSDLTNLKK | | | |
| | ELNWLKEPDKFSLQNALKDLDNAYKKFFKEKA | | | |
| | GFPKFKSKKINRFSYKTNFTNGNIMYCGQHIKLP | | | |
| | KLGMVKVRDKQVPKGRILNATISKEPSGRYYVS | | | |
| | LCCTDVDIEVFENTNNHIGLDLGIKEFCISSCGEF | | | |
| | IENPKYLKKSLNKLAKLQRELSRKTIGSLNRNKT | | | |
| | RLKVARLQEHIANQRNDFLQKLSTKLIKENDIIC | | | |
| | IEDLQVKNMIRNRKLSRLISDVSWSEFIRQLEYK | | | |
| | ANWHGRQIVKVGKFFASSQICNKCGYKNEEVK | | | |
| | DLNIREWICPSCNETHDRDINASRNILKEGLRLIT | | | |
| | IQNK | | | |
| CdiTnpB5 | VAVVEKAYKFRMYPNKKQQELINKTFGCCRFV | 46 | — | |
| | YNKYLAKRIEVYKNNKETFTYKQCSSDLTNLK | | | |
| | KELKWLKEPDKFSLQNALKDLDNAYKKFFKEK | | | |
| | SGFPKFKSKKINRFSYKTNFTNGNIMYFSQHIKL | | | |
| | PKLGMVKIRDKQVPQGRILNATISKEPSGRYYV | | | |
| | SLCCTDVDIEAFENTNNQIGLDLGIKEFCISSCGD | | | |
| | FIENPKYLKKSLSKLAKLQRELSRKTIGSLNRNK | | | |
| | ARLKVARLQEHIANQRNDFLQKLSTKLIKENDII | | | |
| | CIEDLQVKNMIRNRKLSRLISDVSWSEFIRQLKY | | | |
| | KANWHGRQIVKVGKFFASSQICNKCGYKNEEV | | | |
| | KDLNVREWICPSCNETHDRDINASINILKEGLRL | | | |
| | ITIQNK | | | |
| CdiTnpB6 | VVVVEKAYKFRMYPNKKQQELINKTFGCCRFV | 47 | — | |
| | YNKYLAKRIDVYKNNKETFTYKQCSSDLTNLK | | | |
| | KELKWLKEPDKFSLQNALKDLDNAYKKFFKEK | | | |
| | TGFPKFKSKKINRFSYKTNFTNGNIMYCGQHIKL | | | |
| | PKLGMVKIRDKQVPQGRILNATISKEPSGRYYV | | | |
| | SLCCTDVDIEAFENTNNHIGLDLGIKEFCISSCGE | | | |
| | FIENPKYLKKSLNKLAKLQRELSRKTIGSLNRNK | | | |
| | ARLKVARLQEHIANQRKDFLQKLSTKLIKENDII | | | |
| | CIEDLQVKNMIKNHKLSRSISDVSWSEFIRQLEY | | | |
| | KANWHGRQIVKVGKFFASSQICNKCGYKNEEV | | | |
| | KNLNIREWICPSCNETHDRDINASINILKEGLRLI | | | |
| | TIQNK | | | |
| CdiTnpB7 | VIAVEKAYKFRMYPNKKQQELINKTFGCCRFV | 48 | — | |
| | YNKYLAKRIDVYKNDKETFTYKQCSSDLTNLK | | | |
| | KELNWLKEPDKFSLQNALKDLDNAYKKFFKEK | | | |
| | AGFPKFKSKKINRFSYKTNFTNGNIMYCGQHIK | | | |
| | LPKLGMVKIRDKQVPQGRILNATISKEPSGRYY | | | |
| | VSLCCTDVDIEAFENTNNQIGLDLGIKEFCISSCG | | | |
| | EFIENPKYLKKSLNKLAKLQRELSRKTIGSLNRN | | | |
| | KARLKVARFQEHIANQRNDFLQKLSTKLIKEND | | | |
| | IICIEDLQVKNMIRNRKLSRLISDVSWSEFIRQLE | | | |
| | YKANWYGRQIVKVGKFFASSQICNKCGYKNEE | | | |
| | VKDLNIREWICPSCNETHDRDINASINILKEGLR | | | |
| | LITIQNK | | | |
| CdiTnpB8 | VIVVEKAYKFRMYPNKKQQELINKTFGCCRFV | 49 | — | |
| | YNKYLAKRIDVYKNDKETFTYKQCSSDLTNLK | | | |
| | KELNWLKEPDKFSLQNALKDLDNAYKKFFKEK | | | |
| | AGFPKFKSKKINRFSYKTNFTNGNIMYCGQHIK | | | |
| | LPKLGMVKVRDKQVPKGRILNATISKEPSGRYY | | | |
| | VSLCCTDVDIEVFENTNNHIGLDLGIKEFCISSCG | | | |
| | EFIENPKYLKKSLNKLAKLQRELSRKTIGSLNRN | | | |
| | KTRLKVARLQEHIANQRNDFLQKLSTKLIKEND | | | |
| | IICIEDLQVKNMIRNRKLSRLISDVSWSEFIRQLE | | | |
| | YKANWHGRQIVKVGKFFASSQICNKCGYKNEE | | | |
| | VKDLNIREWICPSCNETHDRDINASRNILKEGLR | | | |
| | LITIQNK | | | |
| CdiTnpB9 | VIVVEKAYKFRMYPNKKQQELINKTFGCCRFV | 50 | — | |
| | YNKYLAKRIEVYKNDKETFTYKQCSSDLTNLK | | | |
| | KELKWLKEPDKFSLQNALKDLDNAYKKFFKEK | | | |
| | AGFPKFKSKKINRFSYKTNFTNGNIMYCGQHIK | | | |
| | LPKLGMVKIRDKQVPKGRILNATISKEPSGRYY | | | |
| | VSLCCTDVDIEAFENTNNQIGLDLGIKEFCISSCG | | | |
| | EFIENPKYLKKSLSKLAKLQRELSRKTIGSLNRN | | | |
| | KARLKVARLQEHIANQRNDFLQKLSTKLIKEND | | | |
| | IICIEDLQVKNMIRNRKLSRLISDVSWSEFIRQLK | | | |
| | YKANWHGRQIVKVGKFFASSQICNKCGYKNEE | | | |
| | VKDLNIREWICPSCNETHDRDINASINILKEGLR | | | |
| | LITIQNK | | | |

TABLE 6
Minimal IS elements for the CboIStron element

| | | | Sequence |
|---|---|---|---|
| | | | derived |
| Element | Nucleotide sequence | Sequence derived from LE | from RE |
| Minimal | TAATTAAACTGTTATAACAAATCTAAA | TAATTAAACTGTTATAACAAATCTA | GTATATAT |
| IS element | CAAAACTATACAAATATGTATAGAAAA | AACAAAACTATACAAATATGTATAG | TTTTCGTA |
| competent | ATAGATTTATAACAGGAAGCCCTGTGT | AAAAATAGATTTATAACAGGAAGCC | TCGG (SEQ |
| for | ATCATGTGAATGGTGCATACTCGGTGT | CTGTGTATCATGTGAATGGTGCATA | ID NO: 64) |
| efficient | TAATTGCTTTTAACTCCTAAAGCCTTAC | CTCGGTGTTAATTGCTTTTAACTCCT | |
| splicing | AACCAAAGCGAAGTTGGAAACGACAA | AAAGCCTTACAACCAAAGCGAAGTT | |
| | GCGTAACGGTGCGAAAGCTGAAAAAA | GGAAACGACAAGCGTAACGGTGCG | |
| | TAGTAAGGATGGCATAAGCTGAAATA | AAAGCTGAAAAAATAGTAAGGATG | |
| | AAATCCAATTAGGTTCTATAAAAGGGT | GCATAAGCTGAAATAAAATCCAATT | |
| | GGTCGTGACATATAGAAAACGTTGGGT | AGGTTCTATAAAAGGGTGGTCGTGA | |
| | GCTAAATGCTGTAATAATGGATGTTTA | CATATAGAAAACGTTGGGTGCTAAA | |
| | GCAGGGAAATTCCTAAGTCTTTTAAGA | TGCTGTAATAATGGATGTTTAGCAG | |
| | TATGGAAGACCTTCAACGACTATCTCC | GGAAATTCCTAAGTCTTTTAAGATA | |
| | TTGAGGGAGAGTAAAGCCACAAGCCA | TGGAAGACCTTCAACGACTATCTCC | |
| | ACGGTGGAAGAAAAATACCGCATCTCT | TTGAGGGAGAGTAAAGCCACAAGC | |
| | TTTGAGATGAAGATATAGTCTACGCTC | CAACGGTGGAAGAAAAATACCGCA | |
| | ATATGAAAGTATGAGAGGTCTACTCGT | TCTCTTTTGAGATGAAGATATAGTC | |
| | GAGAGAAAGACTGCTATAGGTGTTGCG | TACGCTCATATGAAAGTATGAGAGG | |
| | ACCTATAGTGAATAAGAAAAATATATA | TCTACTCGTGAGAGAAAGACTGCTA | |
| | CAAATATAAAAAATATTGACATATCTC | TAGGTGTTGCGACCTATAGTGAATA | |
| | TCTAAAAAGTGTAATATATTATTATAC | AGAAAAATATATACAAATATAAAA | |
| | TAAAGGGAGGTGAGTACGTATATATTT | AATATTGACATATCTCTCTAAAAAG | |
| | TTCGTATCGG (SEQ ID NO: 58) | TGTAATATATTATTATACTAAAGGG | |
| | | AGGTGAGTAC (SEQ ID NO: 61) | |
| Minimal | TAATTAAACTGTTATAACAAATCTAAA | TAATTAAACTGTTATAACAAATCTA | ATATTTTT |
| IS element | CAAAACTATACAAATATGTATAGAAAA | AACAAAACTATACAAATATGTATAG | CGTATCGG |
| competent | ATAGATTTATAACAGGAAGCCCTGTGT | AAAAATAGATTTATAACAGGAAGCC | (SEQ ID |
| for | ATCATGTGAATGGTGCATACTCGGTGT | CTGTGTATCATGTGAATGGTGCATA | NO: 65) |
| splicing | TAATTGCTTTTAACTCCTAAAGCCTTAC | CTCGGTGTTAATTGCTTTTAACTCCT | |
| | AACCAAAGCGAAGTTGGAAACGACAA | AAAGCCTTACAACCAAAGCGAAGTT | |
| | GCGTAACGGTGCGAAAGCTGAAAAAA | GGAAACGACAAGCGTAACGGTGCG | |
| | TAGTAAGGATGGCATAAGCTGAAATA | AAAGCTGAAAAAATAGTAAGGATG | |
| | AAATCCAATTAGGTTCTATAAAAGGGT | GCATAAGCTGAAATAAAATCCAATT | |
| | GGTCGTGACATATAGAAAACGTTGGGT | AGGTTCTATAAAAGGGTGGTCGTGA | |
| | GCTAAATGCTGTAATAATGGATGTTTA | CATATAGAAAACGTTGGGTGCTAAA | |
| | GCAGGGAAATTCCTAAGTCTTTTAAGA | TGCTGTAATAATGGATGTTTAGCAG | |
| | TATGGAAGACCTTCAACGACTATCTCC | GGAAATTCCTAAGTCTTTTAAGATA | |
| | TTGAGGGAGAGTAAAGCCACAAGCCA | TGGAAGACCTTCAACGACTATCTCC | |
| | ACGGTGGAAGAAAAATACCGCATCTCT | TTGAGGGAGAGTAAAGCCACAAGC | |
| | TTTGAGATGAAGATATAGTCTACGCTC | CAACGGTGGAAGAAAAATACCGCA | |
| | ATATGAAAGTATGAGAGGTCTACTCGT | TCTCTTTTGAGATGAAGATATAGTC | |
| | GAGAGAAAGACTGCTATAGGTGTTGCG | TACGCTCATATGAAAGTATGAGAGG | |
| | ACCTATAGTGAATAAGAAAAATATATA | TCTACTCGTGAGAGAAAGACTGCTA | |
| | CAAATATAAAAAATATTGACATATCTC | TAGGTGTTGCGACCTATAGTGAATA | |
| | TCTAAAAAGTGTAATATATTATTATAC | AGAAAAATATATACAAATATAAAA | |
| | TAAAGGGAGGTGAGTACATATTTTTCG | AATATTGACATATCTCTCTAAAAAG | |
| | TATCGG (SEQ ID NO: 59) | TGTAATATATTATTATACTAAAGGG | |
| | | AGGTGAGTAC (SEQ ID NO: 62) | |
| Minimal | TAATTAAACTGTTATAACAAATCTAAA | TAATTAAACTGTTATAACAAATCTA | AGTCGTGT |
| IS element | CAAAACTATACAAATATGTATAGAAAA | AACAAAACTATACAAATATGTATAG | CATAGTAT |
| competent | ATAGATATCCCAGCGGTCAAAACAGGC | AAAAATAGAT (SEQ ID NO: 63) | AAATGTCT |
| for | GGCAGTAAGGCGGTCGGGATAGTTTTC | | ACATTTAA |
| excision | TTGCGGCCCTAATCCGAGCCAGTTTAC | | ATAGAAAT |
| (with 800- | CCGCTCTGCTACCTGCGCCAGCTGGCA | | GTATATAT |
| bp cargo) | GTTCAGGCCAATCCGCGCCGGATGCGG | | TTTTCGTA |
| | TGTATCGCTCGCCACTTCAACATCAAC | | TCGG (SEQ |
| | GGTAATCGCCATTTGACCACTACCATC | | ID NO: 66) |
| | AATCCGGTAGGTTTTCCGGCTGATAAA | | |
| | TAAGGTTTTCCCCTGATGCTGCCACGC | | |
| | GTGAGCGGTCGTAATCAGCACCGCATC | | |
| | AGCAAGTGTATCTGCCGTGCACTGCAA | | |
| | CAACGCTGCTTCGGCCTGGTAATGGCC | | |
| | CGCCGCCTTCCAGCGTTCGACCCAGGC | | |
| | GTTAGGGTCAATGCGGGTCGCTTCACT | | |
| | TACGCCAATGTCGTTATCCAGCGGTGC | | |
| | ACGGGTGAACTGATCGCGCAGCGGCGT | | |
| | CAGCAGTTGTTTTTTATCGCCAATCCAC | | |
| | ATCTGTGAAAGAAAGCCTGACTGGCGG | | |
| | TTAAATTGCCAACGCTTATTACCCAGC | | |
| | TCGATGCAAAAATCCATTTCGCTGGTG | | |
| | GTCAGATGCGGGATGGCGTGGGACGC | | |
| | GGCGGGGAGCGTCACACTGAGGTTTTC | | |
| | CGCCAGACGCCACTGCTGCCAGGCGCT | | |
| | GATGTGCCCGGCTTCTGACCATGCGGT | | |
| | CGCGTTCGGTTGCACTACGCGTACTGT | | |
| | GAGCCAGAGTTGCCCGGCGCTCTCCGG | | |
| | CTGCGGTAGTTCAGGCAGTTCAATCAA | | |
| | CTGTTTACCTTGTGGAGCGACATCCAG | | |
| | AGGCACTTCACCGCTTGCCAGCGGCTT | | |
| | ACCATCCAGCGCCACCATCCAGTAGTC | | |
| | GTGTCATAGTATAAATGTCTACATTTA | | |
| | AATAGAAATGTATATATTTTTCGTATC | | |
| | GG (SEQ ID NO: 60) | | |

TABLE 7

| Nuclease | | | |
|---|---|---|---|
| | | Vector | |
| | | sequence | |
| | | SEQ ID NO | |
| | | (protein & | |
| | Vector ID, | RNA), E. | |
| | E. coli | coli | Target of the guide RNA in |
| | expression | expression | E. coli experiments (TAM in brackets) |
| GstTnpB1 | pSL4624 | 73 | [TTTAA]GCTGATCGAAAAGCATGAACTGCCTTCTGT |
| | | | GTACTGGGAA (SEQ ID NO: 79) |
| GstTnpB3 | pSL4369 | 74 | [TTTAT]GGACAAGTGGTTCACCATGCGTTGCTTTAT |
| | | | GGTATGATAG (SEQ ID NO: 80) |
| GstTnpB4 | pSL4672 | 75 | [TTTAA]CGGTCATTTTCCCGTGTTTTCGTTCTCTGT |
| | | | CTCCAGCGCA (SEQ ID NO: 81) |
| GstTnpB5 | pSL4673 | 76 | [TTCAT]CGCCCCTCTTGTTTGTTGTGCAAATTGGTC |
| | | | CGGTTCCTTA (SEQ ID NO: 82) |
| GstIscB1 | pSL4164 | 77 | TGCGGAGCCGTTGCAACAAGGGCGCTTGTGTGTAGGG |
| | | | AAG[ATGAA] (SEQ ID NO: 83) |
| GstTnpA | pSL4129 | 78 | N/A |
| | | Vector | |
| | | sequence | |
| | Vector ID, | SEQ ID NO | |
| | mammalian | (guide | |
| | expression | RNA), | |
| | (guide | mammalian | Target of the guide RNA in |
| | RNA) | expression | human cell experiments (TAM in brackets) |
| GstTnpB1 | pSL4934 | 90 | [TTTAA]TAAGCATGGTATAGTG (SEQ ID NO: 95) |
| | | | [TTTAA]TTACATATTTATCAAT (SEQ ID NO: 96) |
| | | | [TTTAA]GCAAGGGCTGATGTGG (SEQ ID NO: 97) |
| | | | [TTTAA]TTAGGTGGGTGGTCAT (SEQ ID NO: 98) |
| | | | [TTTAA]CCCTCCAGTGTATCCA (SEQ ID NO: 99) |
| GstTnpB3 | pSL4940 | 91 | [TTTAT]CAATTACAACTTGACG (SEQ ID NO: 100) |
| | | | [TTTAT]CAGTTTTGGAGGATGT (SEQ ID NO: 101) |
| | | | [TTTAT]GTACATCCTCCAAAAC (SEQ ID NO: 102) |
| | | | [TTTAT]TATTATTGTTTGCAAT (SEQ ID NO: 103) |
| GstTnpB4 | pSL4946 | 92 | [TTTAA]TAAGCATGGTATAGTG (SEQ ID NO: 95) |
| | | | [TTTAA]TTACATATTTATCAAT (SEQ ID NO: 96) |
| | | | [TTTAA]GCAAGGGCTGATGTGG (SEQ ID NO: 97) |
| | | | [TTTAA]TTAGGTGGGTGGTCAT (SEQ ID NO: 98) |
| | | | [TTTAA]CCCTCCAGTGTATCCA (SEQ ID NO: 99) |
| GstTnpB5 | pSL4952 | 93 | [TTCAT]GCAGGTGCTGAAAGCC (SEQ ID NO: 104) |
| | | | [TTCAT]CCTAGCAACTTCTCTG (SEQ ID NO: 105) |
| | | | [TTCAT]TTTCCAGCTCTAGAAG (SEQ ID NO: 106) |
| | | | [TTCAT]CAATTAGCCCATCACA (SEQ ID NO: 107) |
| | | | [TTCAT]TCAACAAATATTTACT (SEQ ID NO: 108) |
| GstIscB1 | pSL4925 | 94 | GGCTTTCAGCACCTGC[ATGAA] (SEQ ID NO: 109) |
| | | | CTTCTAGAGCTGGAAA[ATGAA] (SEQ ID NO: 110) |
| | | | GCCTTCTAGAGCTGGAAA[ATGAA] |
| | | | (SEQ ID NO: 111) |
| | | | TGTGATGGGCTAATTG[ATGAA] (SEQ ID NO: 112) |
| | | | AGTAAATATTTGTTGA[ATGAA] (SEQ ID NO: 113) |
| | | | GCTCATAGTAAATATTTGTTGA[ATGAA] |
| | | | (SEQ ID NO: 114) |

TABLE 8
Plasmids

| Plasmid ID | Description | SEQ ID NO: |
|---|---|---|
| pSL0007 | Empty Vector | 115 |
| pSL0008 | Empty Vector | 116 |
| | Empty Vector | 117 |
| pSL4873 | Mock excised ISGst1 | 118 |
| pSL4872 | ΔtnpA ISGst1 | 119 |
| pSL4675 | tnpA + ISGst1 | 120 |
| pSL4735 | tnpA (Y125A) + ISGst1 | 121 |
| pSL4676 | tnpA + ISGst2 | 122 |
| pSL4677 | tnpA + ISGst3 | 123 |
| pSL4678 | tnpA + ISGst4 | 124 |
| pSL4674 | tnpA + ISGst5 | 125 |
| pSL4736 | tnpA (Y125A) + ISGst2 | 126 |
| pSL4685 | tnpA + ISGst2 (leading) | 127 |
| pSL4680 | tnpA + ISGst2 left end del. | 128 |
| pSL4681 | tnpA + ISGst2 right end del. | 129 |
| pSL4738 | TAM mutant TTTAT to GGGCG | 130 |
| pSL4818 | ISGst2 TAM to IS608 TAM | 131 |
| pSL4819 | ISGst2 left end guide to IS608 left end | 132 |
| | guide | |
| pSL4820 | ISGst2 TAM & left end guide to IS608 | 133 |
| pSL4827 | ISGst2 TAM to TEM | 134 |
| pSL4828 | ISGst2 TEM to TAM | 135 |
| pSL2698 | Hpy-tnpA+ IS608 mini-Tn | 136 |
| pSL4028 | Hpy-tnpA (Y127A)+ IS608 mini-Tn | 137 |
| pSL4840 | Gst-tnpA + IS608 mini-Tn | 138 |
| pSL4695 | Hpy-tnpA + ISGst1 mini-Tn | 139 |
| pSL4245 | Gst-tnpA + ISGst2 mini-tn | 140 |
| pSL4972 | ISGst2 mini-tn | 141 |
| pSL4624 | ISGst1 TnpB | 73 |
| pSL4369 | ISGst2 TnpB | 74 |
| pSL4672 | ISGst3 TnpB | 142 |
| pSL4673 | ISGst4 TnpB | 76 |
| pSL4164 | ISGst5 IscB | 77 |
| pSL4032 | Non-targeting | 72 |
| pSL4625 | ISGst1 native target | 143 |
| pSL4128 | ISGst2 native target | 144 |
| pSL4670 | ISGst3 native target | 145 |
| pSL4671 | ISGst4 native target | 146 |
| pSL4031 | ISGst5 native target | 147 |
| pSL4712 | ISGst2 native target TAM GGGCG | 148 |
| pSL4831 | ISGst2 native target TAM TTCAC | 149 |
| pSL4832 | ISGst2 native target TAM TTCAT | 150 |
| pSL4833 | ISGst2 native target TAM TTTAC | 151 |
| pSL4713 | ISGst5 native target TAM GGGCG | 152 |
| pSL4834 | ISGst5 native target TAM TTGAC | 153 |
| pSL4835 | ISGst5 native target TAM TTGAT | 154 |
| pSL4836 | ISGst5 native target TAM TTCAC | 155 |
| pSL4518 | ISGst2 tnpB | 156 |
| pSL4664 | ISGst2 tnpB (D196A) | 157 |
| pSL4478 | ISGst5 iscB | 158 |
| pSL4667 | ISGst5 iscB (D59A, H209A,H210A) | 159 |
| pSL4359 | GstTnpB2 Promoter Screen | 160 |
| pSL4360 | GstTnpB2 Promoter Screen | 161 |
| pSL4361 | GstTnpB2 Promoter Screen | 162 |
| pSL4362 | GstTnpB2 Promoter Screen | 163 |
| pSL4363 | GstTnpB2 Promoter Screen | 164 |
| pSL4364 | GstTnpB2 Promoter Screen | 165 |
| pSL4365 | GstTnpB2 Promoter Screen | 166 |
| pSL4366 | GstTnpB2 Promoter Screen | 167 |
| pSL4367 | GstTnpB2 Promoter Screen | 168 |
| pSL4368 | GstTnpB2 Promoter Screen | 169 |
| pSL4370 | GstTnpB2 Promoter Screen | 170 |
| pSL4033 | IS608 TnpB Target | 171 |
| pSL4035 | ISDra2 TnpB Target | 172 |
| pSL4111 | DraTnpB | 173 |
| pSL3858 | HpyTnpB TnpB | 174 |
| pSL4477 | GstTnpB2 LacZ guide | 175 |
| pSL4519 | GstTnpB2 LacZ guide | 176 |
| pSL4520 | GstTnpB2 LacZ guide | 177 |
| pSL4617 | ChIP Seq | 178 |
| pSL4576 | ChIP Seq | 179 |
| pSL4618 | ChIP Seq | 180 |
| pSL4577 | ChIP Seq | 181 |
| pSL1739 | ChIP Seq | 182 |
| pSL4865 | ChIP Seq | 183 |
| pSL4514 | TAM Library IscB | 184 |
| pSL4841 | N6 TAM Library | 67 |
| pSL4529 | GstTnpA expression vector | 185 |
| pSL4534 | Catalytically inactive GstTnpA expression | 186 |
| | vector | |
| pSL4740 | dGstTnpB native target | 187 |
| pSL4672 | E. coli expression GstTnpB4 | 75 |
| pSL4129 | E. coli expression GstTnpA | 78 |
| pSL4745 | Mammalian Expression Vector GstTnpB1 | 84 |
| pSL4746 | Mammalian Expression Vector GstTnpB3 | 85 |
| pSL4747 | Mammalian Expression Vector GstTnpB4 | 86 |
| pSL4748 | Mammalian Expression Vector GstTnpB5 | 87 |
| pSL4744 | Mammalian Expression Vector GstIscB1 | 88 |
| pSL4749 | Mammalian Expression Vector GstTnpA | 89 |
| pSL4934 | Mammalian Expression Vector GstTnpB1 | 90 |
| pSL4940 | Mammalian Expression Vector GstTnpB3 | 91 |
| pSL4946 | Mammalian Expression Vector GstTnpB4 | 92 |
| pSL4952 | Mammalian Expression Vector GstTnpB5 | 93 |
| pSL4925 | Mammalian Expression Vector GstIscB1 | 94 |
| pSL5514 | TnpB expression plasmid from a native | 188 |
| | IStron locus | |
| pSL5517 | Nuclease-dead TnpB expression plasmid | 189 |
| | from a native IStron locus | |
| pSL5412 | 3xFLAG-TnpB expression construct | 190 |
| pSL5772 | Native IStron expression plasmid | 191 |
| pSL5515 | Mini-IS expression plasmid for splicing; | 192 |
| | pDonor for transposition | |
| pSL5735 | TnpA expression plasmid | 193 |
| pSL5736 | Mutant TnpA expression plasmid | 194 |
| pSL5868 | Transposition intermediate for integration | 195 |
| | assays | |
| pSL5820 | wRNA expression vector | 196 |
| pSL5239 | Human codon optimized TnpB with C- | 197 |
| | terminal NLS | |
| pSL5490 | CboTnpB wRNA for expression in human | 198 |
| | cells, with HDV ribozyme at 3′ end | |
| pSL6177 | CboTnpA transposition donor with | 199 |
| | minimal ends | |
| pSL5761 | Expression vector for minimal intron | 200 |
| | showing efficient splicing | |
| pSL5963 | Expression vector for minimal intron | 201 |
| | showing splicing | |
| pSL4902 | TnpB and ωRNA with native target | 68 |
| pSL5002 | TnpB and ωRNA with library target | 69 |
| pSL5004 | TnpB(D190A) and ωRNA with native | 70 |
| | target | |
| pSL4906 | pTarget (Cbo native guide) | 71 |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A engineered system comprising:
a TnpA protein, a TnpB protein, an IscB protein, or a combination thereof, or one or more nucleic acids encoding thereof; and
optionally, at least one guide RNA, or one or more nucleic acids encoding thereof, wherein the at least one guideRNA is complementary to at least a portion of a target nucleic acid,
wherein at least one of the TnpA, TnpB, and IscB protein is derived from Geobacillus stearothermophilus, Clostridium botulinum, Clostridium senegalense or Clostridioides difficile.

2. The system of claim 1, wherein the TnpA protein comprises an amino acid sequence having at least 70% identity to any of SEQ ID NO: 11, 21, 25, and 38-41, the TnpB protein comprises an amino acid sequence having at least 70% identity to any of SEQ ID NOS: 1-4, 6-9, 17, 22-24, 30-37, and 42-50, the IscB protein comprises an amino acid sequence having at least 70% identity to SEQ ID NO: 5 or 10, or a combination thereof.

3. A system comprising:
a TnpA protein comprising an amino acid sequence having at least 70% identity to SEQ ID NO: 11, 21, 25, and 38-41, or a nucleic acid encoding thereof,
a TnpB protein comprising an amino acid sequence having at least 70% identity to any of SEQ ID NOs: 1-4, 6-9, 17, 22-24, 30-37, and 42-50, or a nucleic acid encoding thereof,
an IscB protein comprising an amino acid sequence having at least 70% identity to SEQ ID NO: 5 or 10, or a nucleic acid encoding thereof, or
a combination thereof; and
optionally, at least one guide RNA, or a nucleic acid encoding thereof, wherein the at least one guide RNA is complementary to at least a portion of a target nucleic acid.

4. The system of claim 1, wherein the system comprises a TnpA protein and a DNA nuclease capable of inducing site-specific single or double strand breaks, or one or more nucleic acids encoding thereof.

5. The system of claim 1, wherein the system comprises a TnpA protein and at least one of the TnpB protein or IscB protein, or one or more nucleic acids encoding thereof.

6. The system of claim 1, further comprising at least one guide RNA comprising a scaffold sequence capable of associating with the TnpA, TnpB, IscB protein, or a combination thereof and a guide sequence complementary to at least a portion of a target nucleic acid.

7. The system of claim 6, wherein the at least one guide RNA is provided on an omega RNA.

8. The system of claim 1, wherein the TnpA protein, TnpB protein, and/or IscB protein are at least partially catalytically inactivated, and optionally fused to an effector polypeptide.

9. The system of claim 1, wherein any or all of the TnpA protein, TnpB protein, and IscB protein comprise at least one nuclear localization sequence (NLS).

10. The system of claim 1, further comprising a target nucleic acid and/or donor nucleic acid.

11. The system of claim 10, wherein the donor nucleic acid is flanked by at least one of a left end sequence and a right end sequence.

12. A method for DNA modification comprising contacting a target nucleic acid sequence with a system of claim 1.

13. The method of claim 12, wherein the target nucleic acid sequence is flanked by on the 5′ end by a transposon-adjacent motif (TAM) sequence and, optionally, the 3′ end by a transposon-encoded motif (TEM) sequence.

14. The method of claim 12, wherein the modification comprises cleavage of the target nucleic acid, excision of the target nucleic acid, integration of the donor nucleic acid, or a combination thereof.

15. The method of claim 12, wherein the target nucleic acid sequence is in a cell and the contacting a target nucleic acid sequence comprises introducing the system into the cell.

16. The system of claim 3, further comprising:
at least one guide RNA comprising a scaffold sequence capable of associating with the TnpA, TnpB, IscB protein, or a combination thereof and a guide sequence complementary to at least a portion of a target nucleic acid;
a target nucleic acid; and/or
donor nucleic acid.

17. The system of claim 3, wherein the system comprises:
a TnpA protein; and
a DNA nuclease capable of inducing site-specific single or double strand breaks or at least one of the TnpB protein or IscB protein; or
one or more nucleic acids encoding thereof.

18. The system of claim 3, wherein the TnpA protein, TnpB protein, and/or IscB protein are at least partially catalytically inactivated, and optionally fused to an effector polypeptide.

19. The system of claim 3, wherein any or all of the TnpA protein, TnpB protein, and IscB protein comprise at least one nuclear localization sequence (NLS).

20. A method for DNA modification comprising contacting a target nucleic acid sequence with a system of claim 3.
